(19) United States
(12) Patent Application Publication
(10) Pub. No.: US 2025/0101176 A1
(43) Pub. Date: Mar. 27, 2025

(54) NON-COVALENT SHIELDING POLYMERS

(71) Applicant: POLYPEPTIDE THERAPEUTIC SOLUTIONS, S.L., PATERNA (ES)

(72) Inventors: Carles FELIP LEON, PATERNA (ES); Irene DOLZ PÉREZ, PATERNA (ES); Sergio ESTEBAN PÉREZ, PATERNA (ES); Lidia HERRERA MUÑOZ, PATERNA (ES); Vicent Josep NEBOT CARDA, PATERNA (ES)

(73) Assignee: POLYPEPTIDE THERAPEUTIC SOLUTIONS, S.L., PATERNA (ES)

(21) Appl. No.: 18/291,139

(22) PCT Filed: Jul. 22, 2022
(86) PCT No.: PCT/EP2022/070607

§ 371 Date: Jan. 22, 2024

(30) Foreign Application Priority Data
Jul. 22, 2021 (EP) 21382665.4

(51) Int. Cl. C08G 69/36; A61K 47/64; C12N 15/88

(52) U.S. Cl. CPC C08G 69/36; A61K 47/645; C12N 15/88

(57) ABSTRACT

The present invention relates to new anionic polymers which are useful as shielding for positively charged proteins or polycation-based non-viral vectors for delivery of proteins or active ingredients, including nucleic acids, to cells.

This application claims the benefit of European Patent Application EP21382665.4 filed on Jul. 22, 2021.

### TECHNICAL FIELD

The present disclosure relates to new anionic polymers which are useful as shielding for positively charged proteins or polycation-based non-viral vectors for delivery of proteins or active ingredients, including nucleic acids, to cells.

### BACKGROUND ART

Delivery of active ingredients to a target site located inside the cells requires appropriate delivery carriers, which provide adequate protection and permit the active ingredients to be efficiently delivered to specific tissues within the body. Nevertheless, the delivery carrier must overcome different extracellular and intracellular barriers in order to reach their target sites inside the cell. Although viral vectors are efficient for gene delivery compared to non-viral vectors, their use is accompanied by several risks including toxicity, immunogenicity and limitations in size of the genetic material cargo. Non-viral vectors are generally safer and more convenient for large scale production, although their transfection efficiency is relatively low.

Use of cationic polymers as non-viral synthetic carriers for delivering active ingredients, and more particularly of nucleic acids, to a target cell have attracted considerable attentions.

Cationic polymers, such as polyethylenimine (PEI), poly(β-amino esters), polyamidoamines, chitosan and other polyamines such as PAsp(DET) are capable to form nanoparticles (polycation-based non-viral vectors) carrying and protecting active ingredients, particularly nucleic acids. Thus, these cationic polymers may carry and protect the active ingredient when it enters the cell membrane and finally reaches the nucleus.

Nevertheless, one major reason for limited development of polycation-based non-viral vectors resides in that it is required that the cationic polymer show different functions at different stages of the delivery process. For example, it may be necessary for the vector to have a high amine density to overcome the endosomal membrane barrier since a protonated potential of the nanoparticle could be a cause of endosome destabilization allowing the release in the cytoplasmatic medium. On the contrary, the positively-charged property of the nanoparticle or protein may cause aggregation in the blood stream and non-specific interaction with a negatively-charged serum component, thereby producing a thrombus in the blood capillary or hindering the protein to do its physiological function. This highly positive charged particles (i.e. polyplexes or proteins) have also risk of inducing high cytotoxicity and excess immune response. Additionally, these positively charged nanoparticles may cause severe serum inhibition and are rapidly cleared from the blood, which will hinder their applications in vivo.

A well-known attempt to solve these problems is positive charge shielding by covering the surface of the nanoparticle with polyethylene glycol (PEG). However, the presence of covalently bonded PEG will significantly reduce the transfection efficiency in case of the polyplexes because the neutral surface of the nanoparticles can decrease the cellular uptake efficiency or may also cause activity loss in case of proteins because the hindered space of the active site. because the neutral surface of the nanoparticles can decrease the cellular uptake efficiency. Also it is well known that also the PEG generates anti-PEG antibodies that may cause immunogenicity and allergic reactions.

Accordingly, from what it is known in the field, there is still a need to find new shielding strategies which overcome the above mentioned problems.

### SUMMARY OF THE INVENTION

Thus, the inventors have designed new anionic polymers which are useful as shielding for positively charged proteins or polycation-based non-viral vectors for delivery of proteins or active ingredients, including nucleic acids, to cells with low cytotoxicity, high efficiency, with adequate plasma half-life time, enhanced permeability and retention, high solubility in aqueous solution, high stability due to limited or even completely suppressed aggregation issues in the bloodstream, potential different cell and tissue tropism.

In the context of the present invention, the terms “anionically charged polymer”, “polyanionically charged polymer”, “anionic polymer” or equivalents, refer to polymers that comprises natural or unnatural anionic amino acids which comprises a net negative charge at basic or physiological pH, and may be, for example glutamic acid or aspartic acid, or a combination thereof, i.e. those which has already been rendered anionic once it loses an hydrogen ion, but it also comprise an anionic group that will be neutral once it gains a hydrogen ion. The polypeptides having an anionic group in the side-chain also comprises polypeptide derivatives obtained through peptide bond of known amino acids having acidic side-chains (e.g. glutamic acid, aspartic acid) as well as polypeptides obtained through peptide bond of any amino acid and subsequent substitution in the side-chain to have an anionically charged group.

The term “non-covalent bond”, as used herein, refers to a bond that does not involve the sharing of electrons, but rather involves more dispersed variations of electromagnetic interactions between molecules. The non-covalent bond can be classified into various categories, such as electrostatic interaction, r-interaction, van der Waals forces, hydrogen bonding and hydrophobic effect.

Thus, a first aspect of the present invention relates to an anionic polymer comprising the following formula (Ia) or (Ib), a pharmaceutically acceptable salt thereof, or any stereoisomer or mixtures of stereoisomers, either of the compound of formula (Ia) or (Ib), or of any of its pharmaceutically acceptable salts, comprising homo-polypeptides or random or block or graft co-polypeptides

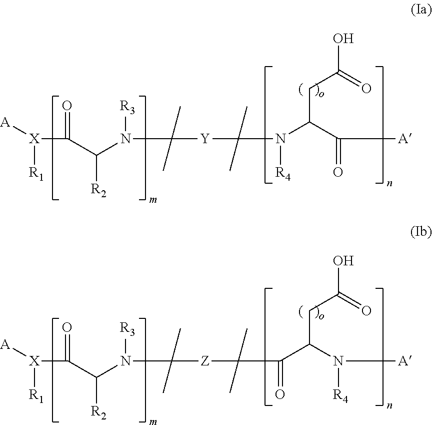

wherein Y is selected from the group consisting of —CO—(CH₂)p-CO— and —CO—(CH₂)q-S—S—(CH₂)r-CO—;
Z is selected from the group consisting of a single bond, —CO—(CH₂)q-S—S—(CH₂)r-NH—(R₅)z-, —CO—(CH₂)p-NH—(R₅)z-, —CO—(CH₂)p-CO—NH—(CH₂)q-S—S—(CH₂)r-NH—(R₅)z-, and —(R₅)z-;
o is an integer selected from 1 to 2;
p, q and r are each independently an integer selected from 1-6;
R₅ is a random or block co-polymer comprising at least two different repeating units selected from the group consisting of (II), (III), (IV), (V), (VI), (VIII), (VIII) and (IX):

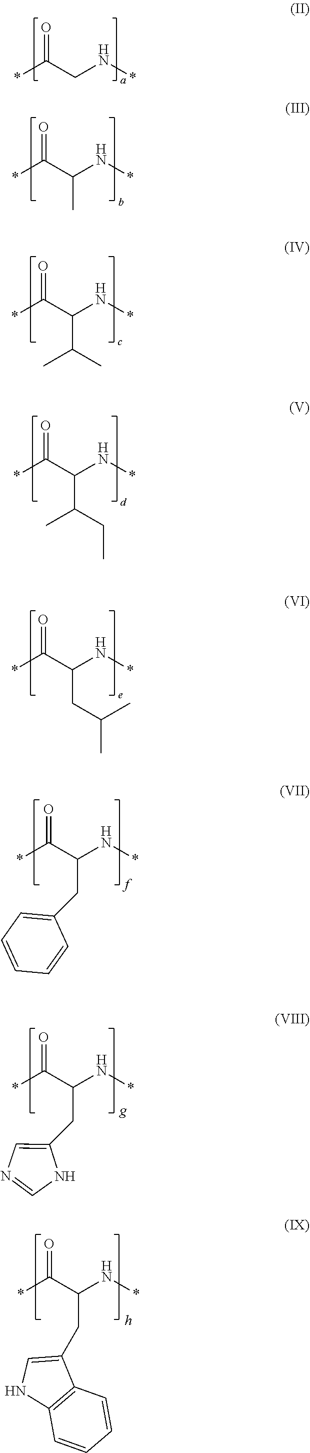

a, b, c, d, e, f, g and h are each independently an integer selected from 0 to 20; with the proviso that at least two of a, b, c, d, e, f, g and h are different to 0;
z is an integer selected from 5 to 100;
X is selected from N, S, and O;
R₁ is selected from the group consisting of H and (C₁-C₆)alkyl, with the proviso that R₁ is absent when X is O;
R₃ and R₄ are each independently selected from the group consisting of H and —CH₃;
m is an integer selected from 5 to 250;
n is an integer selected from 3-200;
with the proviso that the ratio between m:n ranges from 1:8 to 30:1;
R₂ is selected from H, and a radical selected from the group consisting of (X), (XI), (XII), and (XIII)

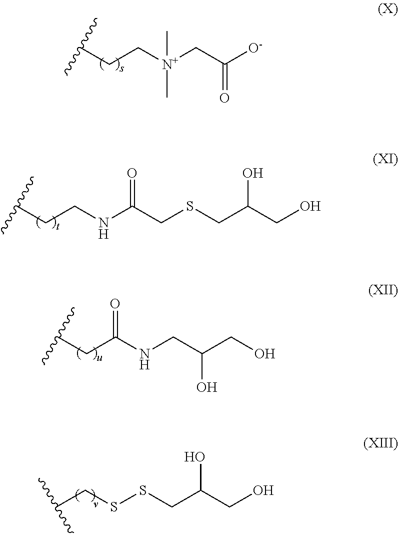

wherein s, t, u, and v are each independently an integer selected from 1 to 4;
wherein “/” indicates that the sequential order of the monomer repeating units defined by square brackets on both sides of the sign is arbitrary, thus though the repeating units defined by square brackets in the formulas Ia and Ib are shown in a particular order for convenience of description, the repeating units may be present in any order and the repeating units may be block or randomly present;
wherein in formula Ib, the sequential order of the respective repeating units of formula (II), (Ill), (IV), (V), (VI), (VII), (VIII) and (IX) of R5 and the repeating units denoted with the square brackets with the n integer, may be block or randomly present;
A and A′ are each independently selected from H, OH, straight chain or branched —(C₁-C₆)alkyl, straight chain or branched —CO(C₁-C₆)alkyl, —(C₅-C₁₀)aryl, —(C₅-C₁₀)heteroaryl, —(C₆-C₁₀)aralkyl, —(C₁-C₆)alkyl-O—(C₅-C₁₀)aryl, —(C₅-C₁₀)heterocycloalkyl, —(C₁-C₁₀)alkoxy, —(C₆-C₁₀)aryloxy, —(C₆-C₁₀)aralkoxy, —(C₅-C₁₀)heteroaralkoxy, —(C₁-C₁₀)alkyl-O—(C₆-C₁₀)aryloxy, an amine protecting group, a natural or unnatural alpha amino acid, a carboxyl protecting group, a labelling or imaging agent and a cell-targeting agent;
being each of A and A′ independently optionally substituted by one or more groups selected from the group consisting of —OH, halogen, —CF₃, —NH₂, —NH—(C₁-C₄)alkyl, —NR₆R₇, —NH—CO—(C₁-C₆)alkyl, —(C₁-C₆)alkyl, —NO₂, —N₃, —CO—(C₁-C₆)alkyl, —CO—O—(C₁-C₆)alkyl, —SO₃H, —SO₂NH₂, —SO₂—N((C₁-C₆)alkyl)₂, —COOH, CONH₂, and —CON((C₁-C₆)alkyl)₂;
R₆ and R₇ are independently selected from the group consisting of H, —(C₁-C₄)alkyl and an amine protecting group.

In accordance with a second aspect of the present invention, there is provided a polymer complex comprising
a) a cationic polymer which covalently or electrostatically bonds with at least one active agent, preferably selected from the group consisting of pharmaceutically active agents, veterinary active agents, cosmetically active agents, diagnostically active agents, nucleic acids, peptides, antibodies, aptamers, proteins, and mixtures thereof; wherein the cationic polymer and the at least one active agent are forming a positively charged nanoparticle; and
b) at least one anionic polymer of formula (Ia) or (Ib) as defined herein which interact electrostatically with the nanoparticle.

In accordance with another aspect of the present invention, there is provided a protein-based complex comprising
a) a positively charged protein, and
b) at least one anionic polymer of formula (Ia) or (Ib) as defined herein which interacts electrostatically with the protein;
   or alternatively
a′) an anionically charged protein covalently or electrostatically bound to a cationic polymer forming a positively charged nanoparticle and
b′) at least one anionic polymer of formula (Ia) or (Ib) according to any of the claims 1-7 which interacts electrostatically with the positively charged nanoparticle of a′).

In accordance with a fourth aspect of the present invention, there is provided a composition comprising of at least one polymer complex or protein-based complex as defined herein, together with one or more appropriate (e.g. pharmaceutically, diagnostically, veterinary or cosmetically) acceptable excipients or carriers.

In a further aspect, the present invention relates to a polymer complex, a protein-based complex or the pharmaceutical composition as defined herein, for use as a medicament.

Alternatively, this aspect may be formulated as a therapeutic product which is the polymer complex as defined herein, or alternatively the protein-based complex as defined herein, or alternatively the composition as defined herein, for use in medicine.

An additional aspect of the present invention relates to the polymer complexes or the pharmaceutical compositions containing them as defined herein
(i) for use as transfection reagent for transfecting at least one active agent into a cell; or
(ii) for use in the in vivo or ex vivo production of biologics encoding a recombinant protein, a peptide or an antibody, or in the production of recombinant virus; or
(iii) for use as a therapeutic or prophylactic vaccine against viral infections or as a therapeutic vaccine against cancers; or
(iv) for use in genome engineering, for cell reprogramming, for differentiating cells or for gene-editing.

An additional aspect of the present invention relates to the protein-based complexes or the pharmaceutical composition containing them as defined herein, for use in protein-based therapy; particularly for use as a therapeutic or prophylactic protein-based vaccine against viral infections or as a therapeutic protein-based vaccine against cancers.

The present invention also provides a device, which may be suitable e.g. for delivering an active agent into a cell, tissue or extracellular space, preferably a nucleic acid or protein; the device comprises the polymer complex or the composition containing them as defined herein. This aspect may also be formulated as a device for use in a method of delivering an active agent, e.g. a nucleic acid or a protein, into a cell, tissue or extracellular space, wherein the device comprises the polymer complex as defined herein, the protein-based complex as defined herein, or the composition containing them as defined herein.

As will be recognized by those of ordinary skill in the art, the appropriate device for delivering an active agent into a cell will depend on the formulation of the composition or pharmaceutical composition that is selected and/or the desired administration site. For example, if the formulation of the composition is appropriate for injection in a subject, the device could be a syringe. For another example, if the desired administration site is cell culture media, the device could be a sterile pipette. For yet another example, if the desired administration site is a vein or artery, the device could be a graft. For yet another example, if the desired administration site is a subcutaneous or organ specific depot, the device could be a surgical implant.

The delivery device of the present invention may be used for a treatment (gene therapy) in which an intended nucleic acid is introduced into a cell responsible for any of various diseases.

In accordance with a further aspect of the present invention, there is provided a method for delivering a protein or a nucleic acid into a target cell, tissue or extracellular space, which comprises administering a solution that contains the polymer complex, the protein-based complex or the composition as defined herein to an animal, including human, so that the complex can be introduced into the target cell, tissue or extracellular space. For the polymer complex or protein-based complex carrying an active ingredient that has its action in the intracellular space, the transport is performed through cell internalization, transferring the complex to the cytoplasm by any internalization mechanism; dissociating the complex inside the cell; and releasing the protein or active ingredient into the cytoplasm. For those protein complexes that have its action in the extracellular space no internalization is aimed or required.

This aspect can be formulated as the use of the polymer complex, the protein-based complex or the composition as disclosed herein, in a delivery method of a nucleic acid into a target cell, which comprises contacting a solution that contains the polymer complex, the protein-based complex or the composition as defined herein, with the target cell, tissue or extracellular space, so that the complex can be introduced into the target cell, tissue or extracellular space; transferring the complex from the endosome to the cytoplasm; dissociating the complex in the cell; and releasing the nucleic acid into the cytoplasm.

Alternatively, this aspect can be formulated as the polymer complex as defined herein, the protein-based complex as defined herein or the composition as defined herein, for use in a method of delivering a nucleic acid or protein into a target cell, tissue or extracellular space which comprises contacting a solution that contains the polymer complex as defined herein, the protein-based complex as defined herein or the composition as defined herein, with the target cell, so that the complex can be introduced into the target cell, tissue or extracellular space; transferring the complex from the endosome to the cytoplasm; dissociating the complex in the cell; and releasing the nucleic acid or protein into the cytoplasm.

An additional aspect relates to a method of transfecting a cell comprising contacting the cell with a polymer complex as defined herein, a protein-based complex as defined herein or the composition as defined herein.

In another aspect, the present disclosure relates to a process for the synthesis of the compounds of formula (Ia) or (Ib) of the first aspect of the disclosure or any embodiment thereto, the process generally comprising polymerizing N-carboxy anhydrides (NCA) of protected or non-protected amino acids known per se, to produce a poly(amino acid), or protected poly amino acid ester, carbamate, S-alkylsulfonyl, or trifluoroacetyl derivative. Then a deprotection step or thiol exchange should be carried out by methods well known for a person skilled in the art. The different radicals present in the repeating units may be introduced at desired ratios by changing the ratios of the respective block or random copolymers.

In accordance with this aspect, related to a process for the synthesis of the compound of formula (I) of the first aspect of the disclosure or any embodiment thereto, the process comprising:
i) reacting an amine or tetrafluoroborate or trifluoroacetate ammonium salt form of an initiator
   i.1) with an appropriate N-carboxyanhydride (NCA); alternatively, reacting the amine or tetrafluoroborate or trifluoroacetate ammonium salt form of initiator of step i) with an appropriate N carboxyanhydrides in a sequential manner to obtain a block co-polymer;
   i.2) alternatively, reacting the amine or tetrafluoroborate or trifluoroacetate ammonium salt form of initiator of step i) with an appropriate NCA mixture in a statistical manner to obtain random co-polymers;
ii) optionally, reacting the amine group at the N-terminal position with an amine reactive group to introduce R1;
iii) optionally, orthogonally removing amino acid side chain protecting groups;
iv) optionally, reacting the amine, thiol, disulfide, S-alkylsulfonyl group or carboxilic acid group at side chain terminal position with an amine or carboxilic acid reactive group to introduce architectural extension, conjugation, labelling or shielding in R2
v) purifying the product obtained in step i), ii) or iii), optionally by fractionation, precipitation, ultrafiltration, dialysis, size-exclusion chromatography, affinity chromatography or tangential flow filtration.

Step i) above may include: a) ring opening polymerization of amino acids N-carboxyanydride (NCA) monomer by reacting the amine or tetrafluoroborate or trifluoroacetate ammonium salt form of initiator with the selected NCA, wherein the ratio of monomer/initiator allows the control of the degree of polymerization (DP); b) a sequential polymerization, wherein block co-polypeptides are prepared following the polymerization reaction a′) in a sequential manner, allowing the first NCA monomer to be consumed and the resulting product may be purified or not before adding the next monomer to build the following polypeptidic block; or c) a statistical polymerization a′) wherein random copolypeptides are prepared following the polymerization reaction in a statistical manner, mixing all the NCA monomers before starting the polymerization by the addition of an amine or tetrafluoroborate or trifluoroacetate ammonium salt form of initiator.

Step ii) above corresponds to the end-capping, wherein the amine group at the N-terminal position is reacted with an amine reactive group to introduce R1.

Step iii) above corresponds to the exchange reaction or deprotection, wherein amino acid side chains are removed orthogonally depending on the protecting group.

Step iv) corresponds to the conjugation, reacting the amine or carboxylic acid group at side chain terminal position to achieve a shielding moiety, by chloracetylation, methylation, thiol exchange, nucleophilic substitution or peptide coupling reactions in a sequential manner if needed.

In accordance with another aspect of the present invention, there is provided a process for preparing a compound which is structurally different from a compound of formula I as defined herein, comprising the following steps:
i. using a compound of formula I as defined herein as starting compound;
ii. making structural changes to the compound of step (i) to obtain a compound, which is structurally different from a compound of formula I.

In a further aspect of the present invention, there is provided the use of a compound of formula I as defined herein, to make a compound, which is structurally different from a compound of formula I.

### BRIEF DESCRIPTION OF THE DRAWINGS

Non-limiting examples of the present disclosure will be described in the following, with reference to the appended drawings, in which

FIG. 1 shows the result of the polyplexes PX_{N1_8_0.5}^{mRNA} (top left), PX_{N1_8_0.5}^{pDNA} (top right), with shielding polymer V1 analyzed by agarose gel electrophoresis technique. This technique shows, in a qualitative manner, the ability of complexation of the polyplexes towards the genetic material (RNA or DNA). Also, it shows the ability to release the genetic material in the presence of low and high concentration of a polyanionic competing agent (heparin) at low and high concentrations. In the lane labeled as M, the free mRNA is seeded and in lane 4 the pDNA is seeded. As can be seen, the free genetic material is shining under the UV transiluminator. In lanes 1 and 5 the polyplex is seeded and it can be seen that when the polycation is present and the polyplex is formed, the genetic material is entrapped and no signal can be observed. In lanes 2 and 6, the polyplex is seeded in presence of low concentration of heparin competitor, showing no release in those conditions. In lanes 3 and 7, the polyplex is seeded together with high concentration of anionic heparin competitor, in this case, the release of the genetic material is observed. This behaviour is the ideal one because the polyplexes need to be stable at low concentrations of competing molecules extracellularly but should be labile enough to release the cargo when an intracellular stimulus is applied.

### DETAILED DESCRIPTION OF THE INVENTION

All terms as used herein in this application, unless otherwise stated, shall be understood in their ordinary meaning as known in the art. Other more specific definitions for certain terms as used in the present application are as set forth below and are intended to apply uniformly through-out the specification and claims unless an otherwise expressly set out definition provides a broader definition.

As used herein, the indefinite articles “a” and “an” are synonymous with “at least one” or “one or more.” Unless indicated otherwise, definite articles used herein, such as “the” also include the plural of the noun.

The term “halogen” or “halo” as used herein means fluorine, chlorine, bromine, and iodine, preferably fluorine, chlorine and bromine, more preferably fluorine and chlorine.

The term “alkenyl” refers to an organic group that is comprised of carbon and hydrogen atoms that contains at least one double covalent bond between two carbons. Typically, an “alkenyl” as used in this disclosure, refers to organic group that contains 2, 3, 4, 5, 6, 7, 8, 9, 10, 11, 12, 13, 14, 15, 16, 17, 18, 19, 20, or 30 carbon atoms, or any range of carbon atoms between or including any two of the foregoing values. It certain instances the alkenyl group will be conjugated, in other cases an alkenyl group will not be conjugated, and yet other cases the alkenyl group may have stretches of conjugation and stretches of non-conjugation. Additionally, if there are more than 2 carbons, the carbons may be connected in a linear manner, or alternatively if there are more than 3 carbons then the carbons may also be linked in a branched fashion so that the parent chain contains one or more secondary, tertiary, or quaternary carbons. An alkenyl may be substituted or unsubstituted.

The term “alkyl”, as used herein refers to a saturated, straight or branched hydrocarbon chain, i.e. it refers to an organic group that is comprised of carbon and hydrogen atoms that contains single covalent bonds between carbons. Typically, an “alkyl” as used in this disclosure, refers to an organic group that contains 1, 2, 3, 4, 5, 6, 7, 8, 9, 10, 11, 12, 13, 14, 15, 16, 17, 18, 19, 20, or 30 carbon atoms, or any range of carbon atoms between or including any two of the foregoing values. Examples of alkyl groups having 1 to 12 carbon atoms may include methyl, ethyl, n-propyl, isopropyl, n-butyl, sec-butyl, tert-butyl, pentyl, n-hexyl, decyl and undecyl group.

Where there is more than 1 carbon, the carbons may be connected in a linear manner, or alternatively if there are more than 2 carbons then the carbons may also be linked in a branched fashion so that the parent chain contains one or more secondary, tertiary, or quaternary carbons. An alkyl may be substituted or unsubstituted.

The term “alkynyl”, refers to an organic group that is comprised of carbon and hydrogen atoms that contains a triple covalent bond between two carbons. Typically, an “alkynyl” as used in this disclosure, refers to organic group that contains that contains 2, 3, 4, 5, 6, 7, 8, 9, 10, 11, 12, 13, 14, 15, 16, 17, 18, 19, 20, or 30 carbon atoms, or any range of carbon atoms between or including any two of the foregoing values. While a C2- alkynyl can form a triple bond to a carbon of a parent chain, an alkynyl group of three or more carbons can contain more than one triple bond. Where there is more than 3 carbons, the carbons may be connected in a linear manner, or alternatively if there are more than 4 carbons then the carbons may also be linked in a branched fashion so that the parent chain contains one or more secondary, tertiary, or quaternary carbons. An alkynyl may be substituted or unsubstituted.

The term generally represented by the notation “Cx-Cy” (where x and y are whole integers and y>x) prior to a functional group, e.g., “C1-C12 alkyl” refers to a number range of carbon atoms. For the purposes of this disclosure any range specified by “Cx-Cy” (where x and y are whole integers and y>x) is not exclusive to the expressed range, but is inclusive of all possible ranges that include and fall within the range specified by “Cx-Cy” (where x and y are whole integers and y>x). For example, the term “C1-C4” provides express support for a range of 1 to 4 carbon atoms, but further provides implicit support for ranges encompassed by 1 to 4 carbon atoms, such as 1 to 2 carbon atoms, 1 to 3 carbon atoms, 2 to 3 carbon atoms, 2 to 4 carbon atoms, and 3 to 4 carbon atoms.

The term “fluoroalkyl” as used herein refers to an alkyl group as defined herein which is substituted one or more times with one or more fluorohalo, preferably perfluorinated.

The term “alkoxy” as used herein refers to an “alkyl-O—” group, wherein alkyl is as defined above.

The term “substituted” means that one or more hydrogen atoms on the designated atom or group are replaced with a selection from the indicated group, provided that the designated atom's normal valency under the existing circumstances is not exceeded. Combinations of substituents and/or variables are permissible.

The term “optionally substituted” means that the number of substituents can be equal to or different from zero. Unless otherwise indicated, it is possible that optionally substituted groups are substituted with as many optional substituents as can be accommodated by replacing a hydrogen atom with a non-hydrogen substituent on any available carbon or nitrogen atom. It is possible that groups in the compounds according to the invention are substituted with one, two, three, four or five identical or different substituents, particularly with one, two or three substituents.

In the embodiments of the invention where the substitution or unsubstitution of a certain group is not specified, i.e., a certain substitution for that group is not indicated, nor is it indicated that the group is unsubstituted, it has to be understood that the possible substitution of this group is the broadest one as defined herein.

The term “disorder” as used herein is intended to be generally synonymous, and is used interchangeably with, the terms “disease,” “syndrome,” and “condition” (as in medical condition), in that all reflect an abnormal condition of the human or animal body or of one of its parts that impairs normal functioning, is typically manifested by distinguishing signs and symptoms.

The term “pharmaceutically acceptable carrier,” “pharmaceutically acceptable excipient,” “physiologically acceptable carrier,” or “physiologically acceptable excipient” as used herein, refers to a pharmaceutically acceptable material, composition, or vehicle, such as a liquid or solid filler, diluent, excipient, solvent, or encapsulating material. Each component should be “pharmaceutically acceptable” in the sense of being compatible with the other ingredients of a pharmaceutical formulation. It should also be suitable for use in contact with the tissue or organ of humans and animals without excessive toxicity, irritation, allergic response, immunogenicity, or other problems or complications, commensurate with a reasonable benefit/risk ratio.

The term “cosmetically acceptable carrier” or “dermatological acceptable carrier” which is herein used interchangeably refers to that excipients or carriers suitable for use in contact with human skin without undue toxicity, incompatibility, instability, allergic response, among others.

The term “therapeutically acceptable” refers to those compounds which are suitable for use in contact with the tissues of patients without excessive toxicity, irritation, allergic response, immunogenicity, are commensurate with a reasonable benefit/risk ratio, and are effective for their intended use.

The term “pharmaceutically, cosmetically or diagnostically acceptable salts”, embraces non-toxic salts commonly used. The preparation of pharmaceutically, cosmetically or diagnostically acceptable salts of the compounds of the invention can be carried out by methods well-known in the art. Generally, such salts can be prepared by reacting the free acid or base form of a compound of the invention with a stoichiometric amount of an appropriate base or acid, respectively, in a suitable solvent such as water, an organic solvent or a mixture of them.

Examples of pharmaceutically, cosmetically or diagnostically acceptable salts include acid addition salts formed with inorganic acids e.g. hydrochloric, hydrobromic, sulfuric, nitric, hydroiodic, metaphosphoric, or phosphoric acid; and organic acids e.g. succinic, maleic, acetic, fumaric, citric, tartaric, benzoic, trifluoroacetic, malic, lactic, formic, propionic, glycolic, gluconic, camphorsulfuric, isothionic, mucic, gentisic, isonicotinic, saccharic, glucuronic, furoic, glutamic, ascorbic, anthranilic, salicylic, phenylacetic, mandelic, embonic (pamoic), ethanesulfonic, pantothenic, stearic, sulfinilic, alginic and galacturonic acid; and arylsulfonic, for example benzenesulfonic, p-toluenesulfonic, oxalic, methanesulfonic or naphthalenesulfonic acid; and base addition salts formed with alkali metals and alkaline earth metals and organic bases such as N,N-dibenzylethylene-diamine, chloroprocaine, choline, diethanolamine, ethylenediamine, meglumine (N-methylglucamine), lysine and procaine; and internally formed salts. The compounds of the invention and their salts may differ in some physical properties, but they are equivalent for the purposes of the present invention.

As used herein, the term “pharmaceutically active agent” refers to and agent that has pharmacological activity and is used for curing, mitigating, treating or preventing a disease in a mammal, in particular a human. The term “cosmetic active agent” refers to an agent that does not provide any therapy but is used for aesthetic purposes, for example to improve the appearance, preserve, condition, cleanse, color or protect the skin, nails or hair.

The term “diagnostic composition” refers to a composition suitable for use in diagnostic, particularly in imaging diagnostic technology. The term “diagnostically effective amount” as used herein, refers to the effective amount of a detection polymer that, when administered, is sufficient for the diagnosis of a disease or disorder; particularly as imaging diagnostic use as contrast imaging agent. The dose of the detection polymer administered will of course be determined by the particular circumstances surrounding the case, including the polymer administered, the route of administration, the particular condition being diagnosticated, and the similar considerations. The diagnostic composition of the present invention comprises one or more diagnostically acceptable excipients or carriers. The term “diagnostically acceptable” refers to that excipients or carriers suitable for use in the diagnosing technology for preparing compositions with diagnostic use; particularly by imaging diagnostic use. The detection of these diagnostic agents in the body of the patient can be carried out by the well-known techniques used such as in imaging diagnostic with magnetic resonance imaging (MRI) and X-ray.

As used herein, the phrase “natural amino acid” refers to the any of the 20 amino acids naturally occurring in proteins. Such natural amino acids include the nonpolar, or hydrophobic amino acids, glycine, alanine, valine, leucine isoleucine, methionine, phenylalanine, tryptophan, and proline. Cysteine is sometimes classified as nonpolar or hydrophobic and other times as polar. Natural amino acids also include polar, or hydrophilic amino acids, such as tyrosine, serine, threonine, aspartic acid (also known as aspartate, when charged), glutamic acid (also known as glutamate, when charged), asparagine, and glutamine. Certain polar, or hydrophilic, amino acids have charged side-chains, depending on environmental pH. Such charged amino acids include lysine, arginine, and histidine. One of ordinary skill in the art would recognize that protection of a polar or hydrophilic amino acid side-chain can render that amino acid nonpolar. For example, a suitably protected tyrosine hydroxyl group can render that tyroine nonpolar and hydrophobic by virtue of protecting the hydroxyl group.

As used herein, the phrase “unnatural amino” refers to amino acids not included in the list of 20 amino acids naturally occurring in proteins, as described above. Such amino acids include the D-isomer of any of the 19 naturally occurring amino acids, glycine is achiral. Unnatural amino acids also include homoserine and ornithine. Other unnatural amino acids side-chains are well know to one of ordinary skill in the art and include unnatural aliphatic side chains. Other unnatural amino acids include modified amino acids, including those that are N-alkylated, cyclized, phosphorylated, acetylated, amidated, azidylated, labelled, and the like.

The terms “treat”, “treating” and “treatment”, as used herein, refers to ameliorating symptoms associated with a disease or disorder, including preventing or delaying the onset of the disease or disorder symptoms, and/or lessening the severity or frequency of symptoms of the disease or disorder.

As used herein, the term “peptide” refers to molecules that comprise two or more consecutive amino acids linked to one another via peptide bonds. The term peptide includes oligopeptides and polypeptides. The term “protein” refers to large peptides, in particular peptides having at least about 50 amino acids. For the purposes of the invention, the terms peptide and protein are used interchangeably.

As used herein, the term “repeating unit”, or “block” refers to a repeating monomeric unit. A repeating unit or a block may consist of a single monomer or may be comprised of one or more monomers, randomly or block, which may be the same or different from each other resulting in a “mixed block”. Thus, the formulae Ia and Ib as defined herein, encompass compounds which may comprise repeating units defined by square brackets wherein each of the monomer units may comprise the same or different substituents. When the monomer units present in the same repeating unit are the same, the repeating unit is a “homopolymer”, whereas when the monomer units present in the same repeating unit comprise different substituents, the repeating unit is a “copolymer”, which may be a “random copolymer” or a “block copolymer”.

For the purposes of the invention, the term “homopolymer” refers to a polymer derived from a single monomer. The term “copolymer” as used herein refers to a polymer derived from more than one monomer. The copolymer may be a random or a block copolymer. The term “random copolymer” as used herein refers to a copolymer in which the monomer units are located randomly in the polymer molecule. The term “block copolymer” as used herein refers to a copolymer that comprises at least two different monomer units that upon polymerization form at least two chemically distinct regions, segments or blocks that are chemically distinguishable from one another. The term block copolymer includes linear block copolymers, multiblock copolymers and star shaped block copolymers.

One skilled in the art will recognize that a repeating unit is defined by square brackets (“[ ]”) depicted around the repeating monomer unit. The number (or letter representing a numerical range) on the lower right of the brackets represents the number of monomer units that are present in the polymer chain.

Using appropriate surface functionality, the compounds of the present disclosure may be further decorated with a cell-targeting group and/or permeation enhancers that can actively target cells and aid in cellular entry, resulting in a conjugate which has improved cell-specific delivery.

As used herein, the term “protective group” is a grouping of atoms that when attached to a reactive group in a molecule masks, reduces or prevents that reactivity. Protective groups for carboxyl and amino groups are described for example in T. W. Green and P. G. M. Wuts, Protective Groups in Organic Chemistry (Wiley, 3rd ed. 1999) in Chapter 5 (pp. 369-451) and Chapter 7 (pp. 495-653), respectively. Suitable amine protecting groups known in the art may be used without limitation and examples thereof include acyl-based groups, carbamate-based groups, imide-based groups, sulfonamide-based groups, and the like. Among them, methyloxycarbonyl, benzyloxycarbonyl, p-methoxybenzyloxycarbonyl, t-butyloxycarbonyl (Boc), 9-fluorenylmethyloxycarbonyl (FMOC), allyloxycarbonyl (Alloc), 2,2,2-trichloroethoxycarbonyl group (Troc), benzoyl (Bz), benzyl (Bn), p-methoxybenzyl (PMB), 3,4-dimethoxybenzyl (DMPM), p-methoxyphenyl (PMP), tosyl (Ts), trimethylsilylethyloxycarbonyl (Teoc), benzhydryl, triphenylmethyl (Trityl), (4-methoxyphenyl)diphenylmethyl (MMT), dimethoxytrityl (DMT), and diphenylphosphino groups are preferable.

Introduction and removal of amino protective groups can be carried out by standard methods such as the ones described in T. W. Green and P. G. M. Wuts, Protective Groups in Organic Chemistry, Wiley, 3rd ed. 1999, Chapter 7 (pp. 495-653).

Suitable carboxy protective groups known in the art may be used without limitation. Representative carboxy protective groups include alkyl, aryl or benzyl esters, silyl esters, amides or hydrazides. In a particular embodiment, the carboxy protective group is selected from the group consisting of —(C1-C6)alkyl, benzyl, p-methoxyphenyl, trimethylsilyl and [2-(trimethylsilyl)ethoxy]methyl (SEM).

Introduction and removal of these protective groups can be carried out by standard methods such as the ones described in T. W. Green and P. G. M. Wuts, Protective Groups in Organic Chemistry, Wiley, 3rd ed. 1999, Chapter 5 (pp. 369-451).

The term “initiator”, as used herein, refers to a chemical molecule employed for the initiation of the ring-opening polymerization (ROP) reaction of α-amino acid N-carboxyanhydrides through Normal Amine Mechanism, wherein the initiator is incorporated within the backbone of the resulting polyamino acid. The initiator may contain one or more nucleophilic groups that can initiate the ROP reaction, accordingly, the initiator may be mono- or multifunctional, respectively, resulting in one or several terminal X groups in the polymer of the invention, respectively.

The term “moiety” refers to a specific segment or functional group of a molecule or compound.

As used herein, the term “subject” refers to any mammal, including both human and other mammals.

The term “nanoparticle” as used herein, refers to a particle with at least two dimensions at the nanoscale, particularly with all three dimensions at the nanoscale. Particularly, when the nanoparticle is substantially rod-shaped with a substantially circular cross-section, such as a nanowire or a nanotube, the “nanoparticle” refers to a particle with at least two dimensions at the nanoscale, this two dimensions being the cross-section of the nanoparticle.

As used herein, the term “size” refers to a characteristic physical dimension. For example, in the case of a nanoparticle that is substantially spherical, the size of the nanoparticle corresponds to the diameter of the nanoparticle. In the case of a nanoparticle that is substantially rod-shaped with a substantially circular cross-section, such as as nanowire or a nanotube, the size of the nanoparticle corresponds to the diameter of the cross-section of the nanoparticle. In the case of a nanoparticle that is substantially box-shaped, such as a nanocube, a nanobox, or a nanocage, the size of the nanoparticle corresponds to the maximum edge length. When referring to a set of nanoparticles as being of a particular size, it is contemplated that the set of nanoparticles can have a distribution of sizes around the specified size. Thus, as used herein, a size of a set of nanoparticles can refer to a mode of a distribution of sizes, such as a peak size of the distribution of sizes.

The term “polydispersity index” (PDI) is used as a measure of broadness of molecular weight distribution. The larger the PDI, the broader the molecular weight. PDI of a polymer is calculated as the ratio of weight average (MW) by number average (Mn) molecular weight.

$$Polydispersity index = \frac{Mw}{Mn}$$

The compounds of Formula (Ia) and (Ib) may exist as geometric isomers (i.e. cis-trans isomers), optical isomers or stereoisomers, such as diastereomers, as well as tautomers. Accordingly, it should be understood that the definition of compounds of Formula (Ia) and (Ib) includes each and every individual isomers corresponding to the structural formula (Ia) and (Ib), including cis-trans isomers, stereoisomers and tautomers, as well as racemic mixtures of these and pharmaceutically acceptable salts thereof. Hence, the definition of compounds of Formula (Ia) and (Ib) is also intended to encompass all R- and S-isomers of a chemical structure in any ratio, e.g. with enrichment (i.e. enantiomeric excess or diastereomeric excess) of one of the possible isomers and corresponding smaller ratios of other isomers. In the particular case of amino acids, they may acquire L-configuration or D-configuration.

The compounds of Formula (Ia) and (Ib) may be provided in any form suitable for the intended administration, in particular including pharmaceutically acceptable salts of the compound of Formula (Ia) and (Ib).

Pharmaceutically acceptable salts refer to salts of the compounds of Formula (Ia) and (Ib), which are considered to be acceptable for clinical, veterinary and/or cosmetic use. Typical pharmaceutically acceptable salts include those salts prepared by reaction of the compounds of Formula (Ia) and (Ib) a mineral or organic acid or an organic or inorganic base. Such salts are known as acid addition salts and base addition salts, respectively. It will be recognized that the particular counter-ion or multiple counter-ions forming a part of any salt is not of a critical nature, so long as the salt as a whole is pharmaceutically acceptable and as long as the counter-ion does not contribute undesired qualities to the salt as a whole. These salts may be prepared by methods known to the skilled person.

The term “pharmaceutically, cosmetically or diagnostically acceptable salts”, embraces non-toxic salts commonly used. The preparation of pharmaceutically, cosmetically or diagnostically acceptable salts of the compounds of the invention can be carried out by methods well-known in the art. Generally, such salts can be prepared by reacting the free acid or base form of a compound of the invention with a stoichiometric amount of an appropriate base or acid, respectively, in a suitable solvent such as water, an organic solvent or a mixture of them.

Examples of pharmaceutically acceptable addition salts include acid addition salts formed with inorganic acids e.g. hydrochloric, hydrobromic, sulfuric, nitric, hydroiodic, metaphosphoric, or phosphoric acid; and organic acids e.g. succinic, maleic, acetic, fumaric, citric, tartaric, benzoic, trifluoroacetic, malic, lactic, formic, propionic, glycolic, gluconic, camphorsulfuric, isothionic, mucic, gentisic, isonicotinic, saccharic, glucuronic, furoic, glutamic, ascorbic, anthranilic, salicylic, phenylacetic, mandelic, embonic (pamoic), ethanesulfonic, pantothenic, stearic, sulfinilic, alginic and galacturonic acid; and arylsulfonic, for example benzenesulfonic, p-toluenesulfonic, oxalic, methanesulfonic or naphthalenesulfonic acid; and base addition salts formed with alkali metals and alkaline earth metals and organic bases such as N,N-dibenzylethylenediamine, chloroprocaine, choline, diethanolamine, ethylenediamine, meglumine (N-methylglucamine), lysine and procaine; and internally formed salts.

As used herein, the term “label or imaging” refers to a molecule that facilitates the visualization and/or detection of a targeting molecule disclosed herein. Thus, in the context of the present disclosure the expression, “labeling or imaging agent” refers to any substance that is used as a label, or that enhances specific structures in any imaging technique. An imaging agent, hence, includes optical imaging agent, magnetic resonance imaging agent, radioisotope, and contrast agent. Imaging or labelling agents are well known in the art. Particular examples or imaging or labelling agents are gases such as sterilized air, oxygen, argon, nitrogen, fluorine, perfluorocarbons, carbon dioxide, nitrogen dioxide, xenon and helium; commercially available agents used in positron emission tomography (PET), computer assisted tomography (CAT), single photon emission computerized tomography, x-ray, fluoroscopy, and magnetic resonance imaging (MRI). Examples of suitable materials for use as contrast agents in MRI include the gadolinium chelates currently available, such as diethylene triamine pentacetic acid (DTP A) and gadopentotate dimeglumine, as well as iron, magnesium, manganese, copper and chromium. Examples of materials useful for CAT and x-rays include iodine based materials for intravenous administration, such as ionic monomers typified by diatrizoate and iothalamate, non-ionic monomers such as iopamidol, isohexol, and ioversol, non-ionic dimers, such as iotrol and iodixanol, and ionic dimers, for example, ioxagalte. Other useful materials include barium for oral use and non-soluble salts such as zinc acetate. In some molecules, an imaging agent is a dye. In some molecules, an imaging agent is a fluorescent moiety. In some molecules, a fluorescent moiety is selected from: a fluorescent protein, a fluorescent peptide, a fluorescent dye, a fluorescent material or a combination thereof. Examples of fluorescent dyes include, but are not limited to, xanthenes (e.g., rhodamines, rhodols and fluoresceins, and their derivatives); bimanes; coumarins and their derivatives (e.g., umbelliferone and aminomethyl coumarins); aromatic amines (e.g., dansyl; squarate dyes); benzofurans; fluorescent cyanines; indocarbocyanines; carbazoles; dicyanomethylene pyranes; polymethine; oxabenzanthrane; xanthene; pyrylium; carbostyl; perylene; acridone; quinacridone; rubrene; anthracene; coronene; phenanthrecene; pyrene; butadiene; stilbene; porphyrin; pthalocyanine; lanthanide metal chelate complexes; rare-earth metal chelate complexes; and derivatives of such dyes. Examples of fluorescein dyes include, but are not limited to, 5-carboxyfluorescein, fluorescein-5-isothiocyanate, fluorescein-6-isothiocyanate and 6-carboxyfluorescein. Examples of rhodamine dyes include, but are not limited to, tetramethylrhodamine-6-isothiocyanate, 5-carboxytetramethylrhodamine, 5-carboxy rhodol derivatives, tetramethyl and tetraethyl rhodamine, diphenyldimethyl and diphenyldiethyl rhodamine, dinaphthyl rhodamine, rhodamine 101 sulfonyl chloride (sold under the tradename of TEXAS RED®). Examples of cyanine dyes include, but are not limited to, Cy3, Cy3B, Cy3.5, Cy5, Cy5.5, Cy7, IRDYE680, Alexa Fluor 750, IRDye800CW, ICG. Examples of fluorescent peptides include GFP (Green Fluorescent Protein) or derivatives of GFP (e.g., EBFP, EBFP2, Azurite, mKalama1, ECFP, Cerulean, CyPet, YFP, Citrine, Venus, YPet). Fluorescent labels are detected by any suitable method. For example, a fluorescent label may be detected by exciting the fluorochrome with the appropriate wavelength of light and detecting the resulting fluorescence, e.g., by microscopy, visual inspection, via photographic film, by the use of electronic detectors such as charge coupled devices (CCDs), photomultipliers, etc. In some molecules, the imaging agent is labeled with a positron-emitting isotope (e.g., 18F) for positron emission tomography (PET), gamma-ray isotope (e.g., 99mTc) for single photon emission computed tomography (SPECT), or a paramagnetic molecule or nanoparticle (e.g., Gd3+ chelate or coated magnetite nanoparticle) for magnetic resonance imaging (MRI). In some molecules, the imaging agent is labeled with: a gadolinium chelate, an iron oxide particle, a super paramagnetic iron oxide particle, an ultra small paramagnetic particle, a manganese chelate or gallium containing agent. Examples of gadolinium chelates include, but are not limited to diethylene triamine pentaacetic acid (DTPA), 1,4,7,10-tetraazacyclododecane-1,4,7,10-tetraacetic acid (DOTA), and 1,4,7-triazacyclononane-N,N′,N″-triacetic acid (NOTA). In some molecules, the imaging agent is a near-infrared fluorophore for near-infra red (near-IR) imaging, a luciferase (firefly, bacterial, or coelenterate) or other luminescent molecule for bioluminescence imaging, or a perfluorocarbon-filled vesicle for ultrasound. In some molecules, the imaging agent is a nuclear probe. In some molecules, the imaging agent is a SPECT or PET radionuclide probe. In some molecules, the radionuclide probe is selected from: a technetium chelate, a copper chelate, a radioactive fluorine, a radioactive iodine, a indiuim chelate. Examples of Tc chelates include, but are not limited to HYNIC, DTPA, and DOTA. In some molecules, the imaging agent contains a radioactive moiety, for example a radioactive isotope such as 211At, 131I, 125I, 90Y, 186Re, 188Re, 153Sm, 212Bi, 32P, 64Cu radioactive isotopes of Lu, and others.

The term “cell-targeting agent” refers to any biological or chemical structure displaying affinity for a molecule present in the human or animal body, which are able to direct the functionalized nanoparticles by directing them towards the target site for therapeutic treatment since e.g., it selectively binds to receptors that are expressed or over-expressed on specific cell types. The term therefore includes ligands for specific receptors or antigens, such as antibodies for a specific antigen, folic acid for its receptor or sugars such as galactose for its hepatic receptors. The targeting agent may be attached to the functionalized end-group of the anionic polymer through the A and/or A′ moiety; or it may also be attached to the cationic polymer.

Cell-targeting groups are well known in the art. Examples of targeting agents include, but are not limited to monoclonal and polyclonal antibodies (e.g. IgG, IgA, IgM, IgD, IgE antibodies), sugars (e.g. mannose, mannose-6-phosphate, galactose, galactosamine, mannosamine), proteins (e.g. transferrin), oligopeptides (e.g. cyclic and acylic RGD-containing oligopeptides), oligonucleotides (e.g. aptamers), and vitamins (e.g. folate), a Her-2 binding peptide, TLR agonists, β-D-Glucose, Asn-Gly-Arg peptide, angiopep2, aptamers (A-9, A10, Anti-gp120, TTA1, sgc8, Anti MUC-1, AS1411), primaquine, zidovudine, superoxide dismutase, prednisolone, platinum, cisplatin, sulphamethoxazole, amoxicillin, etoposide, mesalzine, doxorubicin, paclitaxel, 5-amino salicylic acid, denosumab, docetaxel, calcitonin, proanthocyanidin, methotrexate, camptothecin, galactose, glycyrrhetinic acid, lactose, hyaluornic acid, octeotride, lactobionic acid, β-galactosyl moiety, arabino-galactan, chitosan, azo-based poly-phosphazene, azo group and 4-amino-benzyl-carbamate, succinate, 4,4′-dihydroxyazo benzene-3-carboxilic acid, cyclic RGD penta-peptide, Aspartic acid octapeptide, alendronate, transferrin, bisphosphonate adendronate, mono sialoganglioside GM1, gluthatione, E-selectinthioaptamer, poloxamer-407, a urokinase-type plasminogen activator receptor (uPAR) antagonist, a CXCR4 chemokine receptor antagonist, a GRP78 peptide antagonist, an RGD peptide, an RGD cyclic peptide, a luteinizing hormone-releasing hormone (LHRH) antagonist peptide, an aminopeptidase targeting peptide, a brain homing peptide, a kidney homing peptide, a heart homing peptide, a gut homing peptide, an integrin homing peptide, an angiogencid tumor endothelium homing peptide, an ovary homing peptide, a uterus homing peptide, a sperm homing peptide, a microglia homing peptide, a synovium homing peptide, a urothelium homing peptide, a prostate homing peptide, a lung homing peptide e.g. RCPLSHSLICY), laminin receptor binding peptide (e.g. YIGSR) a skin homing peptide, a retina homing peptide, a pancreas homing peptide, a liver homing peptide, a lymph node homing peptide, an adrenal gland homing peptide, a thyroid homing peptide, a bladder homing peptide, a breast homing peptide, a neuroblastoma homing peptide, a lymphona homing peptide, a muscle homing peptide, a wound vasculature homing peptide, an adipose tissue homing peptide, a virus binding peptide, or a fusogenic peptide.

In a particular embodiment, in the anionic polymer as defined herein, m is an integer selected from 5 to 250, preferably from 20 to 160, more preferably from 30 to 150, even more preferably from 50 to 140, particularly preferred from 60 to 125; and n is an integer selected from 3 to 200, preferably from 4 to 100, more preferably from 4 to 40, preferably from 5 to 30, more preferably from 6 to 26, particularly preferred from 8 to 20.

In a particular embodiment, in the anionic polymer as defined herein the ratio between m:n ranges from 1:8 to 30:1; preferably from 1:5 to 10:1, more preferably from 1:1 to 8:1, even more preferably 2:1 to 7:1.

In a particular embodiment, in the anionic polymer as defined herein m is an integer selected from 5 to 250, preferably from 20 to 160, more preferably from 30 to 150, even more preferably from 50 to 140, particularly preferred from 60 to 125; and n is an integer selected from 3 to 200, preferably from 4 to 100, more preferably from 4 to 40, preferably from 5 to 30, more preferably from 6 to 26, particularly preferred from 8 to 20; with the proviso that the ratio between m:n ranges from 1:8 to 30:1; preferably from 1:5 to 10:1, more preferably from 1:1 to 8:1, even more preferably 2:1 to 7:1.

In accordance with a particular embodiment, the anionic polymer is of formula (Ia) or (Ib), R₃ is —CH₃; and R₂ is H.

In accordance with a particular embodiment, the anionic polymer is of formula (Ia) or (Ib), R₃ is —CH₃; R₂ is H; m is an integer selected from 5 to 250, preferably from 20 to 160, more preferably from 30 to 150, even more preferably from 50 to 140, particularly preferred from 60 to 125; and n is an integer selected from 3 to 200, preferably from 4 to 100, more preferably from 4 to 40, preferably from 5 to 30, more preferably from 6 to 26, particularly preferred from 8 to 20; with the proviso that the ratio between m:n ranges from 1:8 to 30:1; preferably from 1:5 to 10:1, more preferably from 1:1 to 8:1, even more preferably 2:1 to 7:1.

In accordance with a particular embodiment, the anionic polymer is of formula (Ia) or (Ib), R₃ is H; and R₂ is selected from the group consisting of (X) and (XII).

In accordance with a particular embodiment, the anionic polymer is of formula (Ia) or (Ib), R₃ is H; and R₂ is selected from the group consisting of (X) and (XII); m is an integer selected from 5 to 250, preferably from 20 to 160, more preferably from 30 to 150, even more preferably from 50 to 140, particularly preferred from 60 to 125; and n is an integer selected from 3 to 200, preferably from 4 to 100, more preferably from 4 to 40, preferably from 5 to 30, more preferably from 6 to 26, particularly preferred from 8 to 20; with the proviso that the ratio between m:n ranges from 1:8 to 30:1; preferably from 1:5 to 10:1, more preferably from 1:1 to 8:1, even more preferably 2:1 to 7:1.

Some preferred examples of anionic polymers according to formula (Ib) are those wherein Z is a single bond, according to formula (Ib1) as depicted below:

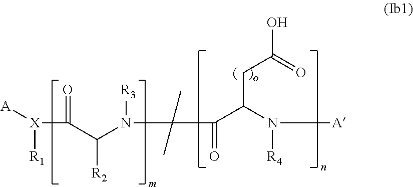

In a particular embodiment, the anionic polymer is of formula (Ib1); and R₂ is selected from the group consisting of a radical selected from the group consisting of (X), (XI), (XII), and (XIII).

In a particular embodiment, the anionic polymer is of formula (Ib1); and R₂ is selected from the group consisting of a radical selected from the group consisting of (X), (XI), (XII), and (XIII); m is an integer selected from 5 to 250, preferably from 20 to 160, more preferably from 30 to 150, even more preferably from 50 to 140, particularly preferred from 60 to 125; and n is an integer selected from 3 to 200, preferably from 4 to 100, more preferably from 4 to 40, preferably from 5 to 30, more preferably from 6 to 26, particularly preferred from 8 to 20; with the proviso that the ratio between m:n ranges from 1:8 to 30:1; preferably from 1:5 to 10:1, more preferably from 1:1 to 8:1, even more preferably 2:1 to 7:1.

In another particular embodiment, the anionic polymer is of formula (Ib1); and R₂ is H.

In another particular embodiment, the anionic polymer is of formula (Ib1); R₂ is H; and R₃ is —CH₃.

In a preferred embodiment, the anionic polymer is of formula (Ib1); R₂ is H; and R₃ is —CH₃; m is an integer selected from 5 to 250, preferably from 20 to 160, more preferably from 30 to 150, even more preferably from 50 to 140, particularly preferred from 60 to 125; and n is an integer selected from 3 to 200, preferably from 4 to 100, more preferably from 4 to 40, preferably from 5 to 30, more preferably from 6 to 26, particularly preferred from 8 to 20; with the proviso that the ratio between m:n ranges from 1:8 to 30:1; preferably from 1:5 to 10:1, more preferably from 1:1 to 8:1, even more preferably 2:1 to 7:1.

The objective of R5 moiety, when present, is to simulate or mimic the well-known GALA peptide (and analogues) activity as a tool for enhancing the transfection activities, more particularly enhancing cellular uptake, membrane penetration and endosomal escape of active agents, and improving cytosolic delivery of the active agent. All of this, results in a significant improvement of the efficiency of transcription/translation processes. GALA peptide has shown to promote the cell internalization by fragmentation, membrane fusion and pore formation as well as membrane-binding properties. Given the key role of the internalization and endosomal escape mechanism for an effective delivery of given cargoes, the approach depicted herein is also based on the non-covalent coating of the GALA-shielding polymer to the nanoparticle surface. When in contact with a cell membrane, the GALA-derived polymer is likely detached from the nanoparticle surface and inserted into the membrane by hydrophobic interactions to form pores that permits the endosomal scape or cytosolic delivery of the nanoparticle cargo or protein.

In a preferred embodiment, the anionic polymer is of formula (Ib), wherein Z is selected from the group consisting of —CO—(CH₂)q-S—S—(CH₂)r-NH—(R₅)z-, —CO—(CH₂)p-CO—NH—(CH₂)q-S—S—(CH₂)r-NH—(R₅)z-, —CO—(CH₂)p-NH—(R₅)z-, and —(R₅)z-; R₅ is a random or block co-polymer comprising at least 2 different repeating units selected from the group consisting of (II), (Ill), (IV), (V), (VI), (VII), (VIII) and (IX); and wherein the sequential order of the respective repeating units of formula (II), (Ill), (IV), (V), (VI), (VII), (VIII) and (IX) of R₅ and the repeating units denoted with the square brackets with the n integer, may be block or randomly present.

In accordance with a particular embodiment, the anionic polymer is of formula (Ib), wherein Z is selected from the group consisting of —CO—(CH₂)q-S—S—(CH₂)r-NH—(R₅)z-, —CO—(CH₂)p-CO—NH—(CH₂)q-S—S—(CH₂)r-NH—(R₅)z-, —CO—(CH₂)p-NH—(R₅)z-, and —(R₅)z-; R₅ is a random or block co-polymer comprising at least 2 different repeating units selected from the group consisting of (Ill), (VI), (VIII) and (IX); wherein c, e, g, and h are independently an integer selected from 0 to 20, with the proviso that at least one, preferably at least two, more preferably at least three, of c, e, g and h are different to 0; and z is an integer selected from 5-35; and wherein the sequential order of the respective repeating units of R₅ and the repeating units denoted with the square brackets with the n integer, may be block or randomly present.

In accordance with a particular embodiment, the anionic polymer is of formula (Ib), wherein Z is selected from the group consisting of —CO—(CH₂)q-S—S—(CH₂)r-NH—(R₅)z-, —CO—(CH₂)p-CO—NH—(CH₂)q-S—S—(CH₂)r-NH—(R₅)z-, —CO—(CH₂)p-NH—(R₅)z-, and —(R₅)z-; R₅ is a random or block co-polymer comprising at least 2 different repeating units selected from the group consisting of (Ill), (VI), (VIII) and (IX); and wherein c, e, g, and h are independently an integer selected from 0 to 20, with the proviso that at least one, preferably at least two, more preferably at least three, of c, e, g and h are different to 0; and z is an integer selected from 5-35; and m is an integer selected from 5 to 250, preferably from 20 to 160, more preferably from 30 to 150, even more preferably from 50 to 140, particularly preferred from 60 to 125; and n is an integer selected from 3 to 200, preferably from 4 to 100, more preferably from 4 to 40, preferably from 5 to 30, more preferably from 6 to 26, particularly preferred from 8 to 20; with the proviso that the ratio between m:n ranges from 1:8 to 30:1; preferably from 1:5 to 10:1, more preferably from 1:1 to 8:1, even more preferably 2:1 to 7:1; and wherein the sequential order of the respective repeating units of R₅ and the repeating units denoted with the square brackets with the n integer, may be block or randomly present.

Some preferred examples of anionic polymers according to formula (Ib) are those wherein Z is (R₅)z, according to formula (Ib2) as depicted below:

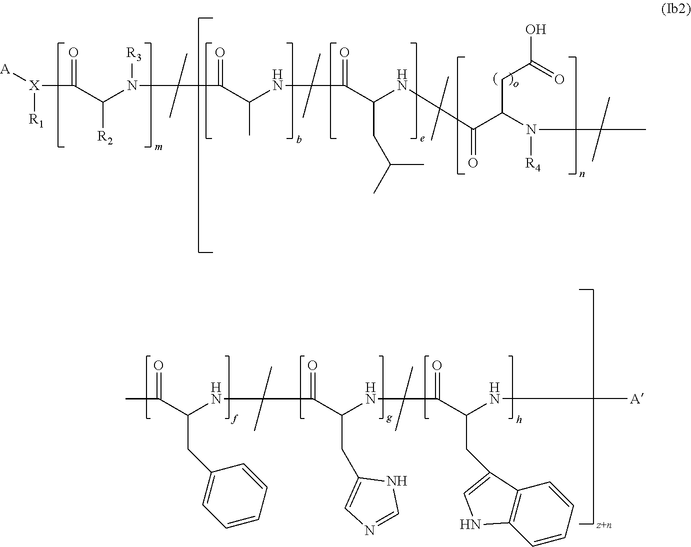

wherein b, e, f, g, and h are each independently an integer selected from 0 to 20; with the proviso that at least two, more preferably at least 3, of b, e, f, g, and h is different to 0;
z is an integer selected from 5 to 100, preferably from 5-35;
and m is an integer selected from 5 to 250, preferably from 20 to 160, more preferably from 30 to 150, even more preferably from 50 to 140, particularly preferred from 60 to 125; and n is an integer selected from 3 to 200, preferably from 4 to 100, more preferably from 4 to 40, preferably from 5 to 30, more preferably from 6 to 26, particularly preferred from 8 to 20; with the proviso that the ratio between m:n ranges from 1:8 to 30:1; preferably from 1:5 to 10:1, more preferably from 1:1 to 8:1, even more preferably 2:1 to 7:1;
wherein “/” between the repeating units defined by square brackets with the b, e, n, f, g and h integers indicates that the sequential order of the monomer repeating units defined by square brackets on both sides of the sign is arbitrary. i.e. they may be present in any order and they may be block or randomly present;
the “/” between the repeating unit defined by square brackets with the m integer and the repeating units defined by the square brackets with the z+n integer, indicates that the sequential order of the monomer repeating units on both sides of the sign is arbitrary, i.e. they may be present in any order and they may be block or randomly present.

In accordance with a particular embodiment, the anionic polymer is of formula (Ia); and R₂ is selected from H, and a radical selected from the group consisting of (X) and (XII).

In another particular embodiment, the anionic polymer is of formula (Ia); R₂ is selected from H, and a radical selected from the group consisting of (X) and (XII); and R₃ is —CH₃.

In a preferred embodiment, the anionic polymer is of formula (Ia); R₂ is selected from H, and a radical selected from the group consisting of (X) and (XII); and R₃ is —CH₃; m is an integer selected from 5 to 250, preferably from 20 to 160, more preferably from 30 to 150, even more preferably from 50 to 140, particularly preferred from 60 to 125; and n is an integer selected from 3 to 200, preferably from 4 to 100, more preferably from 4 to 40, preferably from 5 to 30, more preferably from 6 to 26, particularly preferred from 8 to 20; with the proviso that the ratio between m:n ranges from 1:8 to 30:1; preferably from 1:5 to 10:1, more preferably from 1:1 to 8:1, even more preferably 2:1 to 7:1.

In a preferred embodiment, the anionic polymer is of formula (Ia); Y is —CO—(CH₂)p-CO—; R₂ is selected from H, and a radical selected from the group consisting of (X) and (XII); R₅ and R₄ are H; m is an integer selected from 5 to 250, preferably from 20 to 160, more preferably from 30 to 150, even more preferably from 50 to 140, particularly preferred from 60 to 125; and n is an integer selected from 3 to 200, preferably from 4 to 100, more preferably from 4 to 40, preferably from 5 to 30, more preferably from 6 to 26, particularly preferred from 8 to 20; with the proviso that the ratio between m:n ranges from 1:8 to 30:1; preferably from 1:5 to 10:1, more preferably from 1:1 to 8:1, even more preferably 2:1 to 7:1.

Examples of cationic polymers include poly-L-lysine (PLL), Poly-L-Ornithine (PLO), Poly-L-Histidine, polyamidoamine, polyarginine, poly-[2-{(2-aminoethyl)amino-ethyl-aspartamide](pAsp(DET)), poly(dimethylaminoethyl methacrylate) (pDMAEMA), polyethyleneimine (PEI), chitosan, poly(beta-amino esters), cationic or cationically ionizable lipids or lipid-like materials, polycationic combinations of block, random or graft based on polyaminoacids such as a block co-polymer of polyethylene glycol and polyarginine, a block co-polymer of polyethylene glycol and polylysine, and a block co-polymer of polyethylene glycol and poly-[2-{(2-aminoethyl)amino}-ethyl-aspartamide](PEG-pAsp(DET)), and any other appropriate cationic polymer as long as the polymer can form a polymer complex with the at least one anionic polymer of formula (Ia) or (Ib) of the present invention.

In accordance with some embodiments, at least one anionic polymer of formula (Ia) or (Ib) of the present invention interacts electrostatically with positively charged proteins, acting as a shielding layer on top of the protein, or intercalated among it.

Thus, in accordance with some embodiments, the polymer complex or the protein-based complex as defined herein, may comprise one or more, anionic polymers of formula (Ia) or (Ib). In accordance with some embodiments, the polymer complex or the protein-based complex comprise at least two different anionic polymers of formula (Ia) or (Ib) as defined herein.

In accordance with some embodiments, the anionic polymer comprises a R5 moiety as defined in claim 1, which may be block or randomly present in combination with the repeating units denoted with the square brackets with the n integer.

Some examples according to this embodiment, wherein the anionic polymer is of formula (Ib) and comprises a R₅ moiety which may be block or randomly present in combination with the repeating units denoted with the square brackets with the n integer, may show certain endosomolytic activity.

In certain embodiments, the endosomolytic moiety assumes its active conformation at endosomal pH (e.g., pH 5-6). The “active” conformation is that conformation in which the endosomolytic ligand promotes lysis of the endosome and/or transport of the modular composition of the invention, from the endosome to the cytoplasm of the cell.

As mentioned above, the present invention relates to a polymer complex comprising
a) a cationic polymer which covalently or electrostatically bonds with at least one active agent, preferably selected from the group consisting of pharmaceutically active agents, veterinary active agents, cosmetically active agents, diagnostically active agents, nucleic acids, peptides, antibodies, aptamers, proteins, and mixtures thereof, wherein the cationic polymer and the at least one active agent are forming a positively charged nanoparticle; and
b) at least one anionic polymer of formula (Ia) or (Ib) has defined herein which interacts electrostatically with the nanoparticle.

The skilled person in the art knows different nanoparticulate systems can be used to load active ingredients. These nanoparticulate systems may be in the form of a micelle, a cylindric micelle, a reverse micelle, a vesicle, a lipid-polymer hybrid nanoparticle or a liposome.

In accordance with another aspect of the present invention, there is provided a protein-based complex comprising
a) a positively charged protein, and
b) at least one anionic polymer of formula (Ia) or (Ib) as defined herein which interacts electrostatically with the protein.

In accordance with these aspects of the invention, at least one anionic polymer of formula (Ia) or (Ib) acts as a shielding layer on top of the nanoparticle or the positively charged protein, or alternatively intercalated among it.

That at least one active agent(s) may be covalently bound directly or by one or more linkers, or alternatively that at least one active agent(s) may be non-covalently bound to the compound.

In a preferred embodiment, at least one active agent is covalently linked to the polypeptidic backbone of the cationic polymer through an amino acid side residue, C or N-terminal groups via amide, ester, anhydride bonding or through a linker that include one or more functional groups, including without limitation, alkynes, azides, reactive disulfides, maleimides, hydrazide, hydrazones, Schiff bases, acetal, aldehydes, carbamates, and reactive esters. In an alternative embodiment the covalent link is a bioresponsive one.

In another preferred embodiment, at least one active agent is linked to the polypeptidic backbone of the cationic polymer through electrostatic interaction. Examples of anionic compounds include proteins, polysaccharides, small molecules and nucleic acids.

The conditions for preparation, such as the aqueous medium, pH, temperature and ionic strength may be appropriately adjusted by those skilled in the art.

In accordance with a particular embodiment, the polymer complex is obtained when mixed in aqueous medium at a pH ranging from 4-9, preferably in a pH ranging from 4.5-8.5, more preferably from 5-7.5, being particularly preferred from 6.5-7.4. The pH can be easily adjusted using a buffering solution as the solvent.

In accordance with a particular embodiment, the ionic strength of the solution to be mixed can be appropriately adjusted in a range that does not destroy the structure of the nanoparticles or inhibit encapsulation of the substance to be encapsulated in the nanoparticles, and it is preferably within the range from 0-1000 mM, preferably from 0-300 mM, more preferably from 0-150 mM, being particularly preferred from 0-50 mM.

In accordance with a particular embodiment, the average molecular weight (Mw) of the compounds of formula (Ia) or (Ib) according to the present invention ranges from 200 Da to 80000 Da, preferably from 500 Da to 60000 Da, more preferably from 2000 Da to 40000 Da and more preferably from 2500 Da to 30000 Da, as measured by Gel Permeation Chromatography-Refractive index-Multi Angle Light Scattering-Visible Ultraviolet (GPC-RI-MALS-UV).

In accordance with a particular embodiment, at least one active agent is selected from the group consisting of low molecular weight drugs, peptides, antibodies, hormones, enzymes, nucleic acids, proteins, and combinations thereof.

In accordance with a particular embodiment, the polymer complex (also named herein as polyplex) comprise at least one nucleic acid. In a particular embodiment, the polymer complex comprise a combination of two or more nucleic acids.

As used herein, the term “nucleic acid” refers to DNA or RNA. In a particular embodiment, the nucleic acid is an DNA/RNA hybrid, a short interfering RNA (siRNA), a microRNA (miRNA), a single guide RNA (sgRNA), a donorDNA, a self-amplyfing/replicating RNA, a circularRNA (oRNA), a plasmid DNA (pDNA), a closed-linear DNA (cIDNA), a short hairpin RNA (shRNA), messenger RNA (mRNA), and antisense RNA (aRNA), a messenger RNA (mRNA), a CRISPR guide RNA, an antisense nucleic acid, a decoy nucleic acid, an aptamer, and a ribozyme to name a few, and encompasses both the nucleotide sequence and any structural embodiments thereof, such as double stranded, single stranded, helical, hairpin, etc, and may contain modified or unmodified bases.

When distinct nucleic acids are provided, they may be all DNA molecules or all RNA molecules or may be mixtures of DNA and RNA molecules or molecules comprising an association of DNA and RNA strands.

The nucleic acid may be a poly- or oligonucleotide, such as oligo- or poly-double stranded RNA, oligo- or poly-double stranded DNA, oligo- or poly-single stranded RNA, oligo- or poly-single stranded DNA. Each of the nucleotides contained in the nucleic acid may be a naturally occurring nucleotide or a chemically-modified, non-naturally occurring nucleotide.

The strand length of the nucleic acid is not particularly limited and the nucleic acid may have a short strand ranging from 10 to 200 bases, preferably from 20 to 180 bases, preferably from 25 to 100 bases, preferably from 30 to 50 bases; or the nucleic acid may have a relatively long strand of from 200 to 20000 bases, more preferably of from 250 to about 15000 bases.

In accordance with a particular embodiment, the nucleic acid is closed-linear DNA (cIDNA), i.e. molecules wherein the double stranded region is flanked and protected by two single stranded loops thereby generating dumbbell-shaped molecules.

In a more particular embodiment, the cIDNA consists of a stem region comprising a double stranded DNA sequence of interest covalently closed at both ends by hairpin loops, the cIDNA comprising at least two modified nucleotides.

As used herein, the term “closed linear DNA” or “cIDNA” refers to a single stranded covalently closed DNA molecule that forms a “dumbbell” or “doggy-bone” shaped structure under conditions allowing nucleotide hybridization. Therefore, although the cIDNA is formed by a single stranded DNA molecule, the formation of the “dumbbell” structure by the hybridization of two complementary sequences within the same molecule generates a structure consisting on a double-stranded middle segment flanked by two single-stranded loops. Those skilled in the art knows how to generate cIDNA from open or closed double stranded DNA using routine molecular biology techniques. For instance, those skilled in the art knows that a cIDNA can be generated by attaching hairpin DNA adaptors—for instance, by the action of a ligase—to both ends of an open double stranded DNA. “Hairpin DNA adaptor” refers to a single stranded DNA that forms a stem-loop structure by the hybridization of two complementary sequences, wherein the stem region formed is closed at one end by a single stranded loop and is open at the other end.

A “modified nucleotide” is any nucleotide (e.g., adenosine, guanosine, cytidine, uracil and thymidine) that has been chemically modified—by modification of the base, the sugar or the phosphate group—or that incorporates a non-natural moiety in its structure. Thus, the modified nucleotide may be naturally or non-naturally occurring depending on the modification.

The polymer complexes or the protein-based complexes of the present disclosure constitute a useful tool for therapeutic or diagnostic indications, wherein the compounds of formula (Ia) or (Ib) as defined herein act as a protective shielding for the positively charged nanoparticle bearing the active ingredient or the positively charged protein resulting in an improvement of certain properties such as increase of circulation times, safety or toxicological profile or the release profile in physiological conditions, and, in case of polymer complex, also transfection efficiency to the desired cells,

The polymer complex may have a particle hydrodynamic diameter ranging from 10 nm to 2000 nm, preferably from 20 nm to 800 nm, more preferably from 25 nm to 350 nm, from 30 nm to 300 nm, from 30 nm to 200 nm, as measured by Dynamic Light Scattering instrument.

The protein-based complex may have a particle hydrodynamic diameter ranging from 2 nm to 2000 nm, preferably from 5 nm to 1000 nm, more preferably from 10 nm to 800 nm, from 15 nm to 700 nm, from 20 nm to 600 nm, as measured by Dynamic Light Scattering instrument An additional aspect of the present disclosure relates to a pharmaceutical, diagnostic or theranostic composition comprising at least one polymer complex or at least one protein-based complex as defined above together with one or more appropriate pharmaceutical or diagnostically acceptable excipients.

A further aspect of the disclosure relates to the polymer complex, the protein-based complex or the composition of the disclosure for use as a medicament, in diagnostics or theranostics.

This aspect of the disclosure can be reformulated as the use of the polymer complex, the protein-based complex or the pharmaceutical composition of the disclosure for the manufacture of a medicament.

This aspect could also be formulated as a method for the treatment, diagnostics, prophylaxis and/or theranostics of a disease which comprises administering a therapeutically, diagnostically, prophylactic and/or theranostically effective amount of the polymer complex, the protein-based complex, or the composition of the invention, together with one or more appropriate pharmaceutically, veterinary or cosmetically acceptable excipients and/or carriers, to a subject in need of it, including a human.

In the present invention, the “subject” may be a mammal inclusive of human. The subject may be a healthy subject or a subject affected with some disease.

In the invention, “treatment” refers to curing, preventing or inducing remission of a disease or a disorder or decreasing a progressing speed of a disease or a disorder. The treatment can be attained by administering a therapeutically effective amount of a pharmaceutical composition.

When the method refers to the diagnostics, this aspect could also be formulated as a method for the diagnosis of a disease in an isolated sample of a subject, the method comprises administering to said subject an effective amount of the any of the polymer complex, or pharmaceutical composition having one or more imaging agents as defined above to the isolated sample of the subject. The detection of these imaging agents can be carried out by well-known techniques such as imaging diagnostic techniques. Examples of imaging diagnostic techniques suitable for the present disclosure include, but not limited to, are ultrasound imaging, magnetic resonance imaging (MRI), fluoroscopy, X-ray, positron emission tomography (PET), single-photon emission computed tomography (SPECT), fluorescence microscopy, and in vivo fluorescence.

Thus, the disclosure also refers to the use of the polymer complex or the pharmaceutical composition of the disclosure as a bioimaging tool; particularly to track internalization and delivery of active agents or imaging agents.

As “bioimaging tool” is to be understood according to this description a reagent used in an imaging technique used in biology to trace some compartments of cells or particular tissues. Examples of bioimaging tools include chemiluminescent compounds, fluorescent and phosphorescent compounds, X-ray or alpha, beta, or gamma-ray emitting compounds, etc.

An additional aspect of the present disclosure relates to the use of the polymer complexes as defined herein, as non-viral vectors of general use for biomedical applications, such as vaccines or gene therapy, being effective for transfection of hosts eukaryotic cells in culture, in vivo or ex vivo, monocellular parasites and bacteria, including gene editing using the CRISP/Cas9 methodology.

An additional aspect of the present disclosure relates to the use of the protein-based complexes as defined herein, as carriers of general use in protein-based therapy, such as vaccines.

In a particular embodiment, the present invention refers to the use of the polymer complexes as defined herein, as transfection reagents for delivering active agents (preferably nucleic acids regardless of size and structure, circular and linear nucleic acids) to target cells, in in vivo, in vitro or ex vivo. In a particular embodiment, the active agent is selected from the group consisting of low molecular weight drugs, peptides, proteins, antibodies, nucleic acids, aptamers, and combinations thereof.

Said transfection reagents are also useful for co-transfection of two or more active agents simultaneously, e.g. two or more nucleic acids, simultaneously.

Transfection compositions (such as kits), as well as methods of using the transfection reagents to deliver nucleic acid to target cells are also within the scope of the present invention. Further embodiments will be apparent upon review of the disclosure.

The present invention also relates to a method for in vitro, ex vivo and in vivo transferring active agents comprising using a polymer complex as disclosed herein.

The present invention also provides compositions for use as pharmaceutical compositions for inducing a regulating effect on the expression of one or more target proteins responsible or involved in genetic hereditary diseases or complex genetic diseases, immune diseases, cancers, viral infections in various tissues/organs or tumors.

The present invention also relates to the in vitro or ex vivo use of compositions according to the invention in the production of biologics, in particular biologics encoding a recombinant protein, a peptide or an antibody; or in the production of recombinant virus, such as adeno-associated virus (AAV), lentivirus (LV), adenovirus, oncolytic virus, or baculovirus, or viral or virus-like particles, said compositions comprising a polymer complex as defined herein, comprising at least one nucleic acid molecule for transfection. As used herein, the term “biologics” refers to proteins or nucleic acids or combinations thereof, living entities such as cells or viruses, cell compartments, organoids, and tissues.

The present invention also relates to an in vitro or ex vivo use of the polymer complexes according to the invention for genome engineering, for cell reprogramming, for differentiating cells or for gene-editing.

The compositions for transfecting cells comprise a polymer complex as defined herein and an acceptable excipient, buffering agent, cell culture medium, or transfection medium.

The present invention is also directed to the compositions as defined herein for use as a therapeutic or prophylactic vaccine against viral infections, or a therapeutic vaccine against cancers. Generally, in this aspect, the vaccine is delivered through direct administration such as systemic, intramuscular, intradermal, intraperitoneal, intratumoral, oral, topical, or sub-cutaneous administration, and, in said vaccine, the composition is in association with a pharmaceutically acceptable vehicle. In other words, the vaccine can be injected directly into the body, in particular in a human individual, for inducing a cellular and/or a humoral response.

The cell targeting is achieved through different mechanisms and depends on the nature and properties of the transfection reagent, method or protocol composition or formulation and the route of administration.

In a more particular embodiment, the present invention refers to the polymer complex for use in the prevention and/or treatment of different diseases such as neurodegenerative disorders, neurological diseases, cancer, infectious diseases, disorders related to aging, neuro-inflammation, demyelinating disorder, multiple sclerosis, ischemic disorders, immune disorder, inflammatory disorders, rare diseases, among others depending on the active agent it carries.

The compounds described in the present disclosure, their pharmaceutically acceptable salts and solvates, and the pharmaceutical compositions containing them may be used jointly with other, additional drugs, to provide combined therapy. Said additional drugs may be a part of the same pharmaceutical composition or, alternatively, may be provided in the form of a separate composition for simultaneous or non-simultaneous administration with the pharmaceutical composition comprising a compound with the formula (I), a pharmaceutically acceptable salt, stereoisomer or solvate thereof.

The compounds of the present disclosure may be in crystalline form as free ones or as solvates, and both forms are intended to be included within the scope of the present disclosure. In this regard, the term “solvate”, as used herein, includes both pharmaceutically acceptable solvates, i.e. solvates of the compound with the formula (I) that may be used in the preparation of a medicament, and pharmaceutically unacceptable solvates, which may be useful in the preparation of pharmaceutically acceptable solvates or salts. The nature of the pharmaceutically acceptable solvate is not critical, provided that it is pharmaceutically acceptable. In a particular embodiment, the solvate is a hydrate. The solvates may be obtained by conventional solvation methods that are well-known to persons skilled in the art. Except as otherwise specified, the compounds of the present disclosure also include compounds that differ only in the presence of one or more isotope-enriched atoms. Examples of isotope-enriched atoms, without limitation, are deuterium, tritium, 13C or 14C, or a nitrogen atom enriched in 15N.

One skilled in the art will recognize that a monomer repeating unit is defined by square brackets (“[ ]”) depicted around the repeating monomer unit. The number (or letter representing a numerical value) on the lower right of the brackets represents the number of monomer repeating units that are present in the polymer chain.

In the context of the present disclosure, the term “polyplex” or “polymer complex” refers to a compound formed by electrostatic interaction between a cationic polymer and at least one polyanionic genetic material (preferably a nucleic acid) as described herein. The cationic polymer includes an opposite charge than the polianionic genetic material at the predetermined pH, resulting in the formation of multiple electrostatic bonds between the polianionic genetic material and the polymer at the predetermined pH. The driving force for the polymer complex formation is the multivalency of both polyanionic nucleic acids and polycationic polymers which results in an extremely effective entropically-driven genetic material condensation. The polymer complex (polyplex) containing a nucleic acid is useful as a nonviral synthetic vector capable of delivering the nucleic acid to a target cell. DNA or RNA delivery to a target cell mediated by a nonviral synthetic vector (e.g. a polyplex) has been widely recognized as a promising alternative method for delivery that uses a viral vector which has been confronting significant challenges and drawbacks. These include immunogenic responses (which can prevent redosing), the risk of insertional mutagenesis, the difficulty of large manufacturing at good manufacturing practice grade, limited cargo size, and costsafety issues specific to biological properties.

The term “protein-based complex” refers to a compound formed by electrostatic or combination of hydrophobic and electrostatic interaction between the anionic polymer according to the invention with positively charged proteins at a given pH, acting as a shielding layer on top of the protein, or intercalated among it.

In accordance with a more particular embodiment, the N/P ratio in the polyplexes of the disclosure, which is defined as [total number (N) of cationic groups in the block copolymer]/[total number (P) of phosphate groups in the nucleic acid] is ranging from 1 to 200, preferably from 2 to 100, more preferably from 2 to 50. The N/P ratio means a ratio between the molar concentration (N) of protonable amino groups derived from the side chain of the compound of formula (I) and the molar concentration (P) of phosphate groups derived from the nucleic acid in the mixed solution.

In accordance with a more particular embodiment, the +/− ratio between the cationic polymer or positively charged protein and the at least one shielding polymer of the disclosure, which is defined as [total number positive charges (+) derived from cationic groups in the block copolymer]/[total negative charges (−) derived from polyanionic block in the shielding polymer(s)] is ranging from 0 to 1, preferably from 0.1 to 1, more preferably from 0.3 to 1. The +/− ratio means a ratio between the molar concentration of positive charges (+) derived of protonable amino groups derived from the side chain of the cationic polymer or positively charged protein and the molar concentration of negative charges (−) derived from the anionic block in the mixed solution.

In a more particular embodiment, the polymer complexes may comprise an amount of the at least an active agent in the range of 1 to 50% w/w based on the mass ratio of the active agent to the polymer complex. In a preferred embodiment, the range is of 1 to 30% w/w. In a still more preferred embodiment, the polymer complex comprises an amount of the active agent in the range of 2 to 20% w/w. Other preferred ranges are 2-20% w/w, 3-15% w/w, and 3-7% w/w.

In a more particular embodiment, the protein-based complexes may comprise an amount of the protein in the range of 5 to 99% w/w based on the mass ratio of the active agent to the protein-based complexes. In a preferred embodiment, the range is of 15 to 98% w/w. In a still more preferred embodiment, the protein-based complex comprises an amount of the protein in the range of 30 to 95% w/w. Other preferred ranges are 40-92% w/w, 50-90% w/w, and 60-90 w/w.

The pharmaceutical, diagnostic or theranostic composition according to the disclosure, may be prepared in solid form or aqueous suspension, in a pharmaceutically acceptable diluent. These preparations may be administered by any appropriate administration route, for which reason said preparation will be formulated in the adequate pharmaceutical form for the selected administration route. In a more particular embodiment, administration is performed by oral, topical, rectal or parenteral route (including subcutaneous, intraperitoneal, intradermal, intramuscular, intravenous route, etc.).

It is noted that, in view of the nomenclature used herein for the shielding polymers according to the invention, the numerical values mentioned in parenthesis refer to the degree of polymerization (DP) for each monomeric unit as a statistical number. The DP for a particular monomer unit is calculated dividing the molecular weight of the polymer by the molecular weight of the monomer unit. The DP value is subject to a reasonable uncertainty, due to the ring-opening polymerization mechanism, which, in the context of the present invention, may be considered within the range ±20%, preferably ±15%, more preferably ±10%, even more preferably ±5%, being particularly preferred ±2%. Thus, for example, compound V1 (pSar(72)-b-pGlu(ONa)(9) is described having a pSar DP of 72, a pGlu(ONa) DP of 9; wherein the cited DP numbers are subject to a reasonable uncertainty within the ranges mentioned above.

All terms as used herein in this application, unless otherwise stated, shall be understood in their ordinary meaning as known in the art. Other more specific definitions for certain terms as used in the present application are as set forth below and are intended to apply uniformly through-out the specification and claims unless an otherwise expressly set out definition provides a broader definition.

Throughout the description and claims the word “comprise” and variations of the word, are not intended to exclude other technical features, additives, components, or steps. Furthermore, the word “comprise” encompasses the case of “consisting of”. Additional objects, advantages and features of the invention will become apparent to those skilled in the art upon examination of the description or may be learned by practice of the invention. The following examples and drawings are provided by way of illustration, and they are not intended to be limiting of the present invention. Reference signs related to drawings and placed in parentheses in a claim, are solely for attempting to increase the intelligibility of the claim and shall not be construed as limiting the scope of the claim. Furthermore, the present invention covers all possible combinations of particular and preferred embodiments described herein.

Although only a number of examples have been disclosed herein, other alternatives, modifications, uses and/or equivalents thereof are possible. Furthermore, all possible combinations of the described examples are also covered. Thus, the scope of the present disclosure should not be limited by particular examples, but should be determined only by a fair reading of the claims that follow.

### EXAMPLES

### Example 1: Synthesis of Shielding Block Copolymers PSar-b-PGlu(ONa)

Synthesis of shielding copolymers PSar-b-PGlu(Ona) via block polymerization:

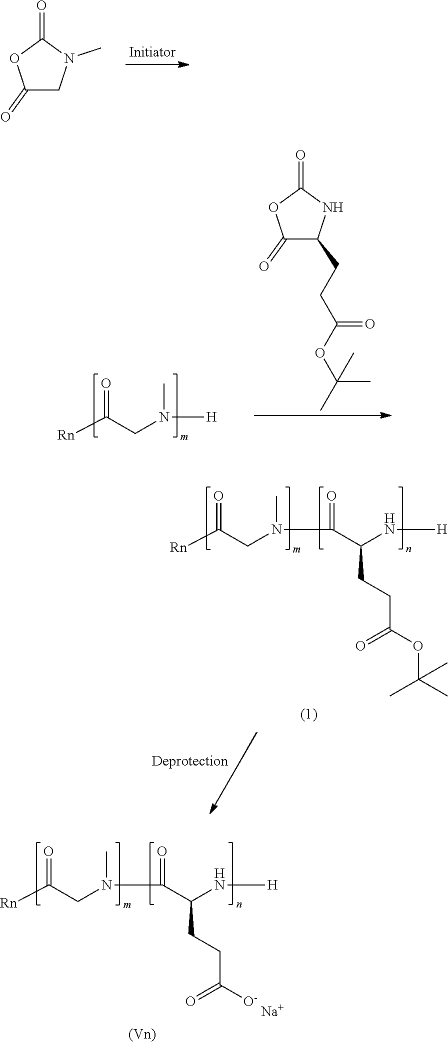

### 1.1A General Procedure for the Polymerization of PSar-b-PGluOtBu

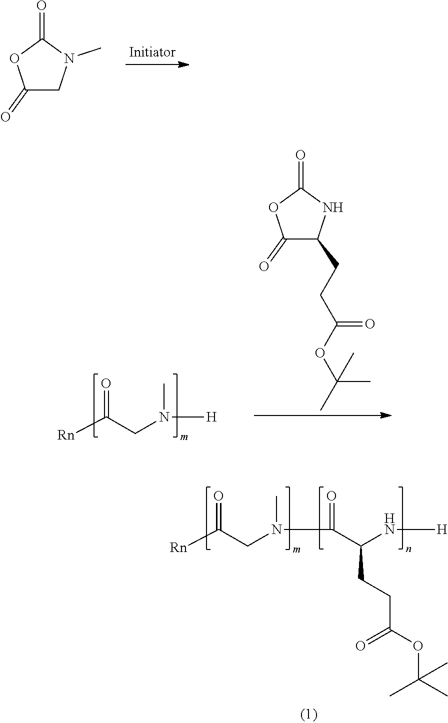

Sarcosine N-carboxyanhydride was added to a Schlenk tube fitted with a stirring bar and a stopper. After 3 cycles of vacuum/N₂, the mixture was dissolved in anhydrous DMF. Then, the initiator (n-Butylamine or iPropylamine) diluted in DMF (2 mL) and was added to the reaction mixture, which was stirred at 10° C. for 16 hours. Once NCA consumption was confirmed by IR the Glutamic acid t-tert butyl ester NCA was added to the reaction mixture dissolved in anhydrous DMF. The mixture was stirred at 10° C. for 16 hours. Upon completion the reaction mixture became clear and full conversion of the monomer could be detected by IR. The reaction mixture was poured into diethyl ether to precipitate the product. The precipitate was isolated by centrifugation (3750 rpm, 4 min) and dried under vacuum. Block copolymer was isolated as a white solid.

Yield: 70-90%. The polymerization product was not characterized by NMR since it is only soluble in the deprotection solvent.

### 1.2A General Procedure for Deprotection Step:

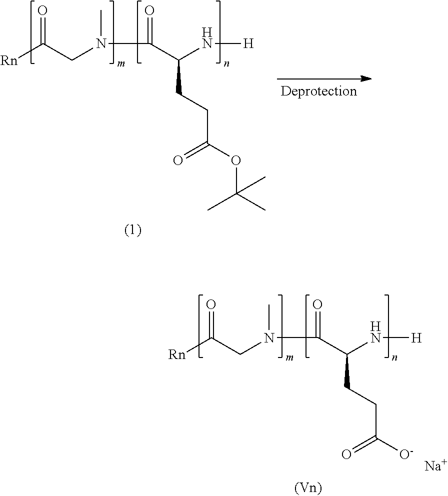

The block copolymer of PSar-b-PGluOtBu was dissolved in trifluoroacetic acid at 0° C. (100 mg/mL) and the mixture was stirred at 5° C. for 1 hour. The reaction mixture was poured into diethyl ether to precipitate the product. The precipitate was isolated by centrifugation (3750 rpm, 4 min) and dried under vacuum. The block copolymer was isolated as a white solid.

Yield: 95%.

[V1-V2]1H NMR (D2O): δ 1.19 (d, J=7.2 Hz, CH3), 1.90-2.53 (m, CH2), 2.83-3.28 (m, CH3), 3.95-4.63 (m, CH2 PSar+CH GluOtBu).

[V3]1H NMR (D2O): δ 1.19 (t, J=7.2 Hz, CH3), 1.35 (m, CH2), 1.70-2.53 (m, CH2), 2.82-3.32 (m, CH3), 4.02-4.60 (m, CH2 PSar+CH GluOtBu).

TABLE 1
Differents block copolymers based on PSar-b-PGlu(ONa) (Vn)

| | | DP (m) | DP (n) for | Mw by SEC | |
|---|---|---|---|---|---|
| Code | Initiator | for Sar^{a} | Glu(ONa)^{a} | (KDa)^{b} | PDI |
| V1 | ⁱPropylamine | 72 | 9 | 6.6 | 1.07 |
| V2 | ⁱPropylamine | 125 | 8 | 8.9 | 1.03 |
| V3 | Butylamine | 23 | 95 | 13.8 | 1.409 |

^{a}Determined by NMR.
^{b}Determined by SEC
V1 = iPr-PSar(72)-b-pGlu(ONa)(9)
V2 = iPr-PSar(125)-b-PGlu(ONa)(8)
V3 = nBu-PSar(23)-b-PGlu(ONa)(95)

wherein the cited DP numbers are subject to a reasonable uncertainty within the range ±20%.

### Example 2. Synthesis of Shielding Copolymers PSar-b-PGlu(Ona) Via Peptide Coupling of Two Homopolymers

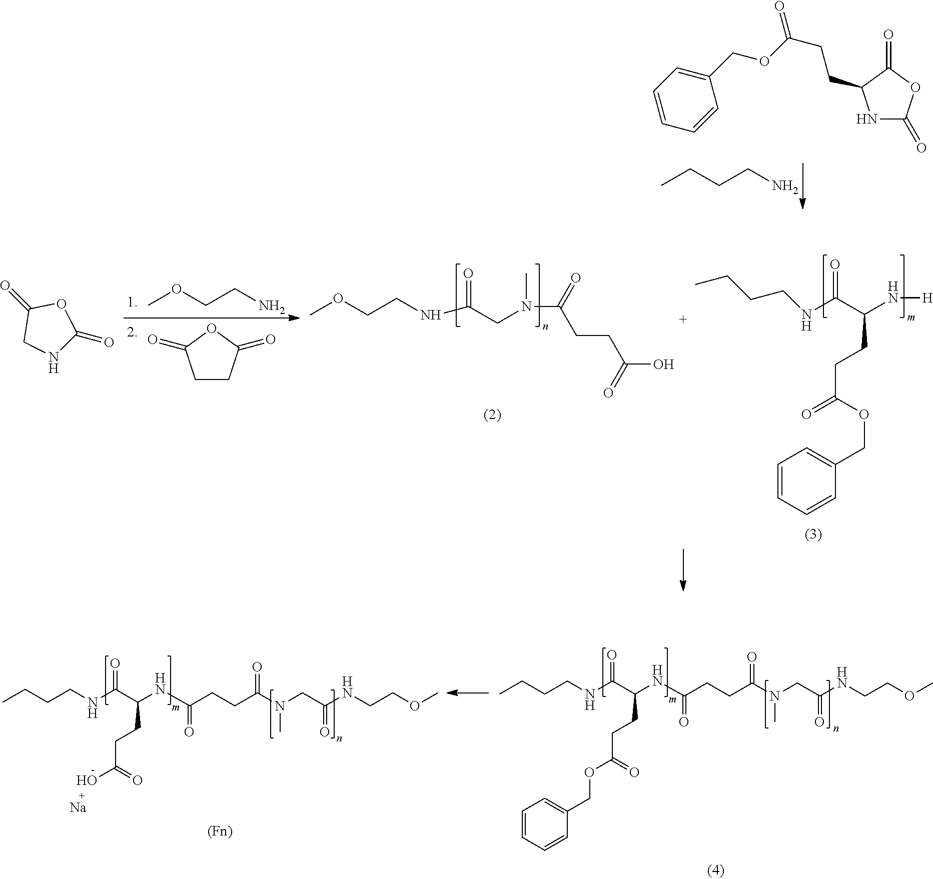

2.1 General Procedure for the Ring Opening Polymerization of MeA-PSar-Succ and nBu-PGluOBzl

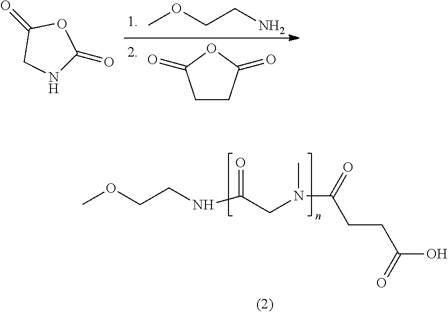

Sarcosine NCA was added to a Schlenk tube fitted with a stirring bar and a stopper. After 3 cycles of vacuum/N2, the mixture was dissolved in anhydrous DMF. Then, the initiator (2-methoxyethylamine) diluted in DMF (2 mL) and was added to the reaction mixture, which was stirred at 10° C. for 16 hours. Once NCA consumption was confirmed by IR. Succinic anhydride (10 eq) in powder was added to the reaction mixture and was stirred for 16 hours at room temperature. The reaction mixture was poured into diethyl ether to precipitate the product. The precipitate was isolated by centrifugation (3750 rpm, 4 min) and dried under vacuum. Homopolymer was isolated as a white solid.

Yield: 70-90%. 1H NMR (300 MHz, TFA): δ 2.71 (brs, 2H, CH₂), 2.86-3.22 (m, 3H, CH₃), 3.40 (s, 3H, CH₃), 4.10-5.58 (m, 2H, CH₂).

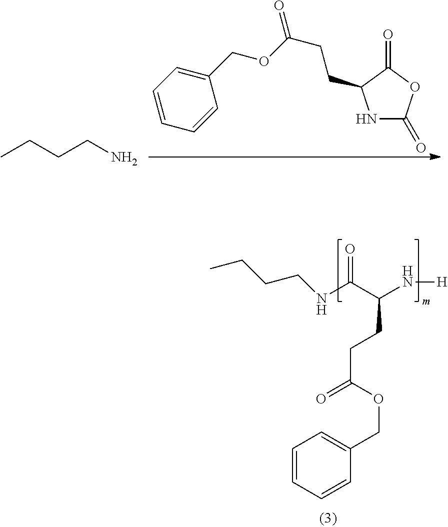

benzyl-L-glutamate NCA was added to a Schlenk tube fitted with a stirring bar and a stopper. After 3 cycles of vacuum/N₂, the mixture was dissolved in anhydrous DMF. Then, the initiator (n-Butylamine) diluted in DMF (2 mL) and was added to the reaction mixture, which was stirred at 10° C. for 16 hours. Once NCA consumption was confirmed by IR. Succinic anhydride (10 eq) in powder was added to the reaction mixture and was stirred for 16 hours at room temperature. The reaction mixture was poured into diethyl ether to precipitate the product. The precipitate was isolated by centrifugation (3750 rpm, 4 min) and dried under vacuum. Homopolymer was isolated as a white solid.

Yield: 70-90%. 1H NMR (300 MHz, TFA): δ 0.87 (t, J=7.3 Hz, CH₃); 1.29 (dd, J=7.2, 15.0 Hz, CH₂), 1.46 (d, J=8.0 Hz, CH₂), 1.80-2.73 (m, CH₂), 4.67 (m, CH), 5.00-5.24 (m, benzyl CH), 7.15-7.37 (brs, aryl CH):

TABLE 2

| | DP (n) for | Mw by SEC | |
|---|---|---|---|
| Code | Sar o GluOBzl^{a} | (Da)^{b} | PDI |
| 2 | 71 | 5200 | 1.05 |
| 3 | 14 | 3100 | N/A |

^{a}Determined by NMR.
^{b}Estimated MW by SEC of the precursor.

2.2 General Procedure for the Peptide Coupling Reaction that Generates the Copolymer of MeA-PSar-Succ-PGluOBzl-nBu:

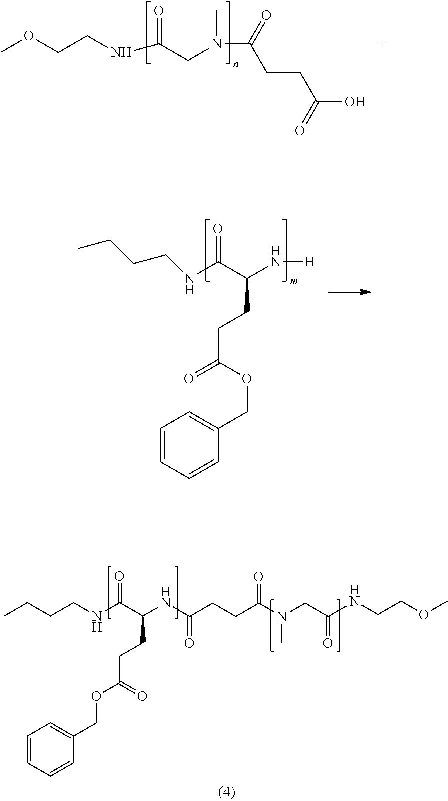

MeA-PSar-succ 0.32 mmol) was added to a two-necked round bottom flask fitted with a stirring bar and a stopper, then purged with 3 cycles of vacuum/N2, and dissolved in 25 mL of DMF. Then, CDI (2.1 eq, 0.68 mmol) was added to the reaction mixture and stirred for 30 minutes at room temperature. After this time, the nBu-PBG (1.2 eq, 0.38 mmol) dissolved in 8 mL of DMF was added. The mixture was stirred at room temperature for 16 hours. The reaction mixture was poured into acetone to precipitate the product. Precipitate was isolated by centrifugation (3750 rpm, 4 min) and dried under vacuum. The final product was isolated as a white solid.

Yield: 60-70. 1H NMR (DMSO): δ 1.06 (t, J=7.3 Hz, CH₃), 1.48 (dd, J=7.4, 14.8 Hz, CH2), 1.65 (dd, J=7.1, 14.4 Hz, CH2), 2.01-2.83 (m, CH₂), 3.17-3.55 (m, CH₃), 3.72 (s, CH3), 3.81-4.06 (m, CH2), 4.44-4.94 (m, CH) 5.19-5.43 (m, benzyl CH), 7.24-7.61 (m, aryl CH).

TABLE 3

| | DP (n) | DP (n) for | Mw by SEC | |
|---|---|---|---|---|
| Code | for Sar^{a} | GluOBzl^{a} | (Da)^{b} | PDI |
| 4 | 67 | 14 | 5610 | 1.02 |

^{a}Determined by NMR.
^{b}Estimated MW by SEC of the precursor.

### 2.3 General Procedure for Deprotection Step:

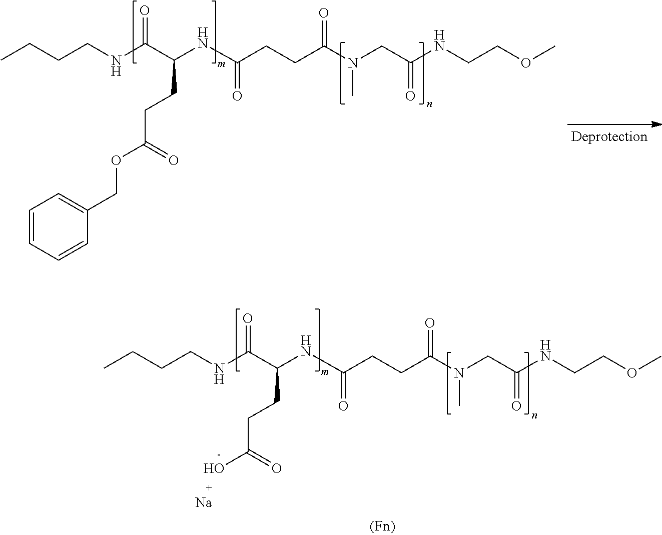

The block copolymer of MeA-PSar-succ-PBG-nBu was dissolved in THE (200 mg/mL). The solution of NaOH aq was added to the mixture and stirred at 4° C. for 16 hours. The reaction mixture is neutralized to pH 4 with 6M HCl and was poured into diethyl ether to precipitate the product. The precipitate was isolated by centrifugation (3750 rpm, 4 min) and dried under vacuum. The block copolymer was isolated as a white solid.

Yield: 70-80%. [V_1]1H NMR (D2O): δ 0.91 (t, J=7.0 Hz, CH₃), 1.33 (d, J=6.8 Hz, CH₂), 1.50 (d, J=6.8 Hz, CH₂), 2.39 (m, CH₂), 2.72-2.81 (m; CH₂), 2.88-3.25 (m, CH₃), 3.39 (s, CH₃), 3.46 (m, CH₂), 3.56 (m, CH₂), 4.03-4.63 (m, CH₂ PSar+CH GluOtBu).

TABLE 4

| | DP (n) | DP (n) for | Mw by SEC | |
|---|---|---|---|---|
| Code | for Sar^{a} | GluONa^{a} | (Da)^{b} | PDI |
| Fn | 67 | 14 | 7100 | N/A |

^{a}Determined by NMR.
^{b}Estimated MW by SEC of the precursor.
N/A not available
F1 = iPr-PGlu(ONa)(14)-Succ-PSar(67)-MeA

### Example 3. Synthesis of Shielding Block Copolymers Rn-PSar-b-PGlu(ONa) with Directing Agent

Shielding block copolymers of Rn-PSar-b-PGlu(ONa) are synthesized using small organic molecules as initiators for polymerization. With these specific organic motifs (TLR7, Galactosamine and manosamine) it will be able to target specific organs.

3.1 Synthetic Route for the Shielding Block Copolymers Rn-PSar-b-PGlu(ONa) with a Directing Agent.

### 3.1.1 General Procedure for the Polymerization of Directing Agent (Rn)-PSar.

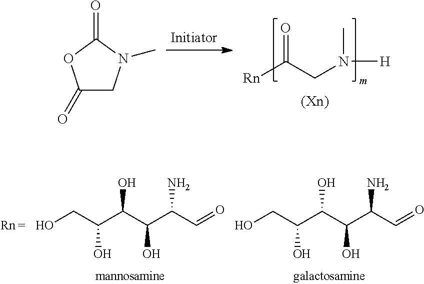

*Note that Mannosamine and galactosamine are shown in its linear form that is en equilibrium with its multiple cyclic forms

Sarcosine N-carboxyanhydride was added to a Schlenk tube fitted with a stirring bar and a stopper. After 3 cycles of vacuum/N₂, the mixture was dissolved in anhydrous DMSO. Then, the initiator (TLR7, galactosamine or mannosamine) diluted in DMSO (2 mL) and was added to the reaction mixture, which was stirred at room temperature for 16 hours. Once NCA consumption was confirmed by IR. The reaction mixture was poured into diethyl ether:THF (8:2) to precipitate the product. The precipitate was isolated by centrifugation (3750 rpm, 4 min) and dried under vacuum. Polysarccosine was isolated as a white solid. Yield: 70%.

[X1-X2-X3-X4]1H NMR (D₂O): δ 2.84-3.25 (m, CH₃), 3.91 (s, CH₃), 4.00 (brs, CH₂), 4.10-4.65 (m, CH₂).

The presence of sugar is performed by the Benedict test. The Benedict test is a chemical test that can be used to detect the presence of reducing sugars in a given analyte. Therefore, simple carbohydrates containing a free ketone or aldehyde functional group can be identified.

Benedict's test is perfomed by heating the reducing sugar with Benedict's reagent (blue solution prepared mixing copper sulfate pentahydrate, sodium citrate and sodium carbonate in distilled water). If color of the solution changes from blue to red, it indicates the presence of sugar in the polymer.

TABLE 5
Targeted shielding Sarcosine precursors according to formula (Xn)

| | | DP (m) | Test | Mw by SEC | |
|---|---|---|---|---|---|
| Code | Initiator (Rn) | for Sar^{a} | Benedict^{a} | (KDa)^{b} | PDI |
| X1 | Mannosamine | 74 | POSITIVE | 5.7 | 1.108 |
| X2 | Mannosamine | 89 | POSITIVE | 6.6 | 1.118 |
| X3 | Galactosamine | 65 | POSITIVE | 6.4 | 1.083 |
| X4 | Galactosamine | 71 | POSITIVE | 7.4 | 1.089 |

^{a}Determined by NMR.
^{b}Determined by SEC
X1 = Mannosamine-PSar(74)
X2 = Mannosamine-PSar(89)
X3 = Galactosamine-PSar(65)
X4 = Galactosamine-PSar(71)

wherein the cited DP numbers are subject to a reasonable uncertainty within the range ±20%.

### 3.2 General Procedure for the Synthesis of Block Copolymer Directing Agent-Rn-PSar-b-PGluONa.

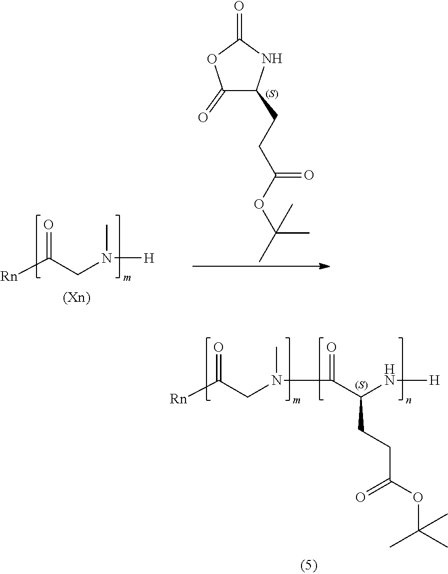

Glutamic acid t-tert butyl ester NCA was added to a Schlenk tube fitted with a stirring bar and a stopper. After 3 cycles of vacuum/N₂, the mixture was dissolved in anhydrous DMF. Then, the PSarcosine (synthesized in the previous section) diluted in DMF (4 mL) and was added to the reaction mixture, which was stirred at 10° C. for 16 hours. Once NCA consumption was confirmed by IR. The reaction mixture was poured into diethyl ether to precipitate the product. The precipitate was isolated by centrifugation (3750 rpm, 4 min) and dried under vacuum. Block copolymer was isolated as a white solid. Yield: 80%.

[5.1-5.2-5.3-5.4]1H-NMR (TFA): δ 2.29-2.56 (s, CH₃), 2.86-3.69 (m, CH₂), 3.77-4.33 (m, CH₃), 4.97 (brs, CH₂), 5.22-5.78 (m, CH₂ PSar+CH GluOtBu).

TABLE 6
Different block copolymer based on Rn-PSar-b-PGluOtBu

| | | DP for | DP for | Mw by GPC | |
|---|---|---|---|---|---|
| Code | Initiator (Rn) | Sar^{a} (n) | Glu(OtBu)^{a} (m) | (Da)^{b} | PDI |
| 5.1 | Mannosamine | 95 | 8 | 8474 | 1.137 |
| 5.2 | Mannosamine | 115 | 9 | 10030 | 1.159 |
| 5.3 | Galactosamine | 93 | 7 | 8122 | 1.151 |
| 5.4 | Galactosamine | 112 | 9 | 9814 | 1.170 |

^{a}Determined by NMR.
^{b}Determined by SEC
5.1 = Mannosamine-PSar(95)-b-PGlu(OtBu)(8)
5.2 = Mannosamine-PSar(115)-b-PGlu(OtBu)(9)
5.3 = Galactosamine-PSar(93)-b-PGlu(OtBu)(7)
5.4 = Galactosamine-PSar(112)-b-PGlu(OtBu)(9)

wherein the cited DP numbers are subject to a reasonable uncertainty within the range ±20%.

### 3.3 General Procedure for Deprotection Step:

Deprotection was carried out following the procedure described above in example 1, generating the corresponding shielding block copolymers Rn-PSar-b-PGlu(ONa) with a directing agent. Yield: 90%.

[A1-A2-A3-A4]1H-NMR (D₂O): 1.67-2.46 (m, CH₂); 2.74-3.23 (m, CH₃); 3.71 (brs, CH2), 3.94 (s, CH₂), 4.01-4.56 (m, CH₂ PSar+CH PGA).

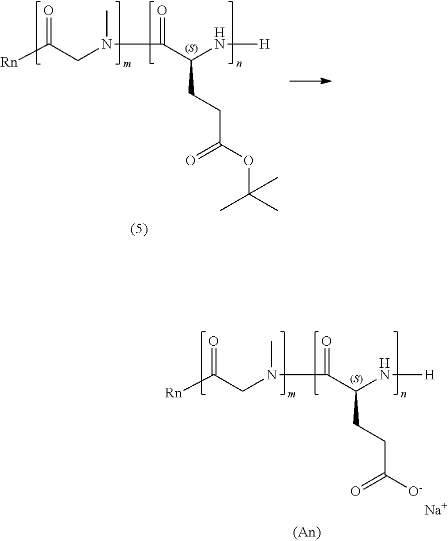

TABLE 7
Targeted shielding diblock copolymers according to formula (An)

| | | DP (m) | DP (n) | Mw by SEC | |
|---|---|---|---|---|---|
| Code | Initiator (Rn) | for Sar^{a} | for Glu^{a} | (KDa)^{b} | PDI |
| A1 | Mannosamine | 90 | 10 | 7849 | 1.046 |
| A2 | Mannosamine | 113 | 10 | 9542 | 1.034 |
| A3 | Galactosamine | 90 | 9 | 7722 | 1.066 |
| A4 | Galactosamine | 114 | 8 | 9329 | 1.054 |

^{a}Determined by NMR.
^{b}MW calculated from GPC analysis of the precursor
A1 = Mannosamine-PSar(90)-b-PGlu(ONa)(10)
A2 = Mannosamine-PSar(113)-b-PGlu(ONa)(10)
A3 = Galactosamine-PSar(90)-b-PGlu(ONa)(9)
A4 = Galactosamine-PSar(114)-b-PGlu(ONa)(8)

wherein the cited DP numbers are subject to a reasonable uncertainty within the range ±20%.

### Example 4. Synthesis of Shielding Block Copolymers PGlu-Diol-b-PGlu(ONa)

### 4.1 General Procedure for the Polymerization of PGluOtBu

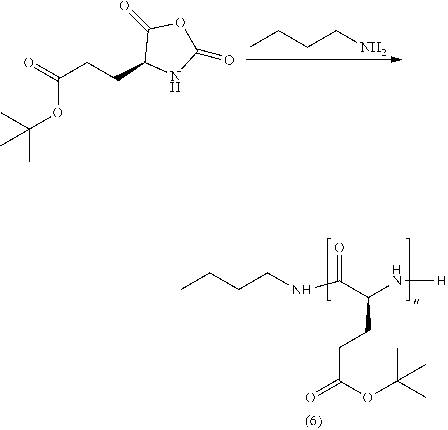

Glutamic acid t-tert butyl ester NCA was added to a Schlenk tube fitted with a stirring bar and a stopper. After 3 cycles of vacuum/N2, the mixture was dissolved in anhydrous DMF. Then, the initiator (n-Butylamine) diluted in DMF (2 mL) and was added to the reaction mixture, which was stirred at 10° C. for 16 hours. Once NCA consumption was confirmed by IR. The reaction mixture was poured into diethyl ether to precipitate the product. The precipitate was isolated by centrifugation (3750 rpm, 4 min) and dried under vacuum. Homopolymer was isolated as a white solid. Yield: 70-90%.

The polymerization product was used without further characterization or purification, the characterization will be performed when deprotection is carried out.

### 4.2 General Procedure for Deprotection Step:

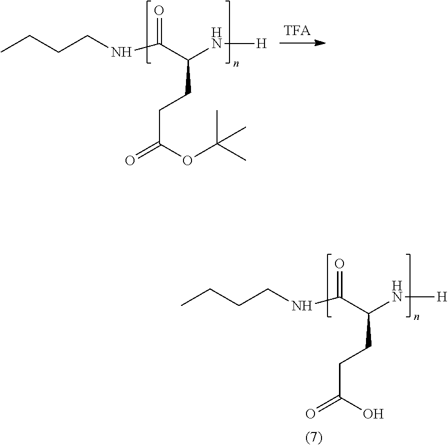

Deprotection was carried out following the procedure described above in example 1, generating the corresponding nBu-PGA.

Yield: 90%. 1H NMR (D2O): δ 0.85 (t, J=7.03 Hz, CH3), 1.28 (m, CH2), 1.44 (m, CH2), 1.84-2.39 (m, CH2), 4.15-4.46 (m, CH).

4.3 General Procedure for the Synthesis of Diblock Copolymer nBu-PGA-b-PGluOtBu.

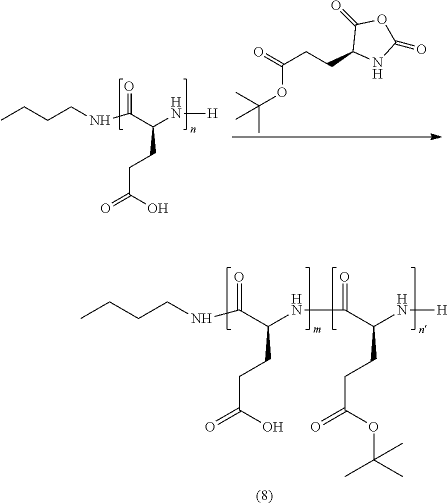

Polymerization reaction was carried out following the procedure described above in example 3.1, using in this case as initiator nBuPGA. Block copolymer was isolated as a white solid. Yield: 70-80%. 1H NMR (TFA): δ 1.62 (t, J=7.59 Hz), 1.99 (m, CH2), 2.09 (m, CH2), 2.12-2.40 (m, CH3), 2.72-3.59 (m, CH2), 5.49 (brs, CH).

4.4 General Procedure for Peptide Coupling with CDI for the Synthesis of PGlu-Diol-b-PGluOtBu:

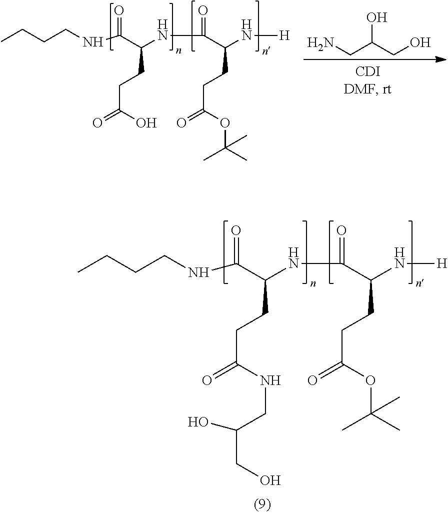

nBuPGA-b-PGluOtBu (3.09 mmol) was added to a two-necked round bottom flask fitted with a stirring bar and a stopper, then purged with 3 cycles of vacuum/N2, and dissolved in 6 mL of DMF. Then, CDI (1.6 eq, 4.95 mmol) was added to the reaction mixture and stirred for 30 minutes at room temperature. After this time, the 3-aminopropane-1.2-diol dissolved in 2 mL of DMF was added. The mixture was stirred at room temperature for 16 hours. The reaction mixture was poured into acetone to precipitate the product. Precipitate was isolated by centrifugation (3750 rpm, 4 min) and dried under vacuum. The final product was isolated as a white solid. Yield: 60-70

1H NMR (DMSO): δ 0.85 (t, J=7.2 Hz, CH₃), 1.20-1.48 (m, CH₃), 1.65-2.31 (m, CH₂), 2.98 (brs, CH), 4.16 (brs, CH), 4.52 (brs, CH), 4.72 (brs, CH), 7.74 (brs, NH-amide), 8.10 (brs, NH amide).

TABLE 8
Characterization of PGluDiol(45)-b-PGlu(OtBu) precursor

| | DP for | DP for | Mw by GPC | |
|---|---|---|---|---|
| Code | GluDiol^{a} (n) | Glu(OtBu)^{a}(n′) | (Da)^{b} | PDI |
| 9.1 | 46 | 4 | 10020 | 1.192 |

9.1 = nBu-PGluDiol(45)-b-PGlu(OtBu)(4)

wherein the cited DP numbers are subject to a reasonable uncertainty within the range ±20%.

### 4.5 General Procedure for Deprotection Step:

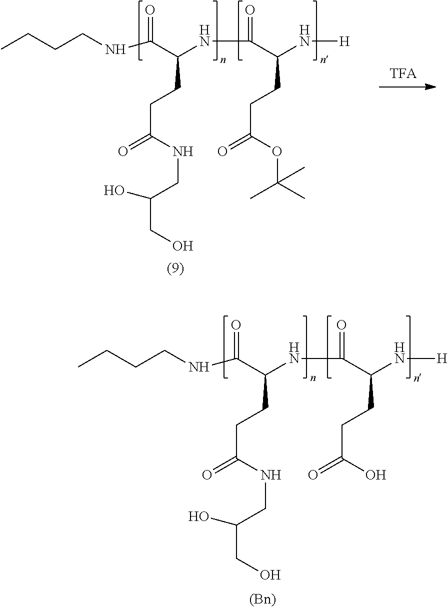

Deprotection was carried out following the procedure described above in example 1, generating the corresponding block copolymers PGlu-Diol-b-PGlu(ONa)

Yield: 90%. 1H NMR (D2O): δ 0.96 (t, J=7.12 Hz, CH3), 1.36 (dd, J=15.0, 7.2 Hz, CH2); 1.54 (d, J=7.12 Hz), 1.88-2.80 (m, CH2), 3.18-3.48 (m, CH2); 3.52-3.74 (m, CH2), 3.83 (brs, CH), 4.37 (brs, CH):

TABLE 9
Targeted shielding diblock copolymers according to formula (Bn)

| | DP (n) for | DP (n′) | Mw by SEC | |
|---|---|---|---|---|
| Code | GluDiol^{a} | for Glu^{a} | (Da)^{b} | PDI |
| B1 | 36 | 6 | 8099 | 1.028 |

^{a}Determined by NMR.
^{b}Determined by SEC
B1 = nBu-PGluDiol(44)-b-PGlu (7)

wherein the cited DP numbers are subject to a reasonable uncertainty within the range ±20%.

### Example 5. Synthesis of Shielding Block Copolymers PSar-R5 (W)

### 5.1 Procedure for the Polymerization of Boc-NH-Ethyl-R5 Random Copolymer.

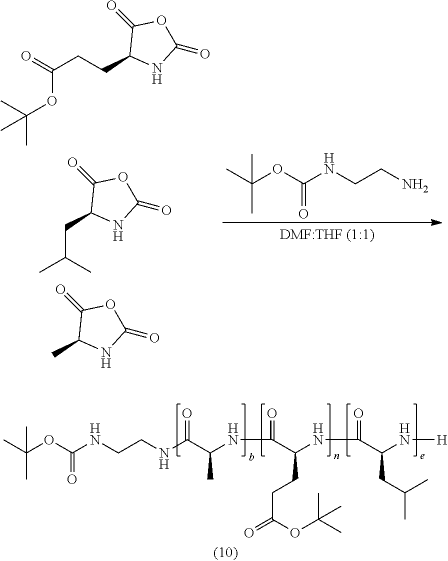

Glutamic acid t-tert butyl ester NCA, Leucine NCA and Alanine NCA were added to a Schlenk tube fitted with a stirring bar and a stopper. After 3 cycles of vacuum/N₂, the mixture was dissolved in anhydrous mixture of DMF:THF (1:1). Then, the initiator (N-Boc-ethylendiamine) diluted in DMF (2 mL) and was added to the reaction mixture, which was stirred at 10° C. for 16 hours. Once NCA consumption was confirmed by IR. The reaction mixture was poured into diethyl ether to precipitate the product. The precipitate was isolated by centrifugation (3750 rpm, 4 min) and dried under vacuum. Random copolymer was isolated as a white solid.

Yield: 70-80%. 1H NMR (300 MHz, TFA): δ 1.51 (d, J=8.9 Hz, CH₃), 1.94-2.22 (m, CH₂), 2.80 (m, CH₂), 3.142 (m, CH₃), 4.05 (brs, CH₂), 4.31 (brs, CH₂), 4.88-5.44 (m, CH).

### 5.2 General Procedure for the One Pot Polymerization/Capping of Boc-NH-Ethyl-R5-b-PSar:

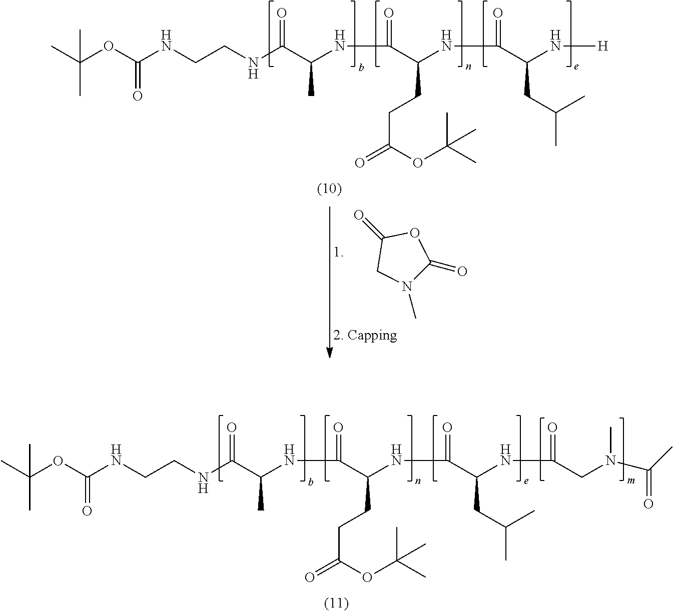

Sarcosine N-carboxyanhydride was added to a Schlenk tube fitted with a stirring bar and a stopper. After 3 cycles of vacuum/N₂, the mixture was dissolved in anhydrous DMF. Then, the initiator (Boc-NH-ethyl-R5 random copolymer) diluted in DMF (2 mL) and was added to the reaction mixture, which was stirred at 10° C. for 16 hours. Once NCA consumption was confirmed by IR acetic anhydride (10 eq, 1.47 mmol) and DIPEA (1 eq, 0.147 mmol) were added to the reaction mixture. Then, the mixture was stirred at 10° C. for 2 hours. The reaction mixture was poured into diethyl ether to precipitate the product. The precipitate was isolated by centrifugation (3750 rpm, 4 min) and dried under vacuum.

Yield: 60-70%. 1H NMR (300 MHz, TFA): δ 1.69 (d, J=9.0 Hz, CH₃), 2.10-2.47 (m, CH₂), 2.97 (m, CH₂), 3.25 (s, CH₃), 3.40 (brs, CH₂), 3.75-4.09 (m, CH₃), 5.05-5.41 (m, CH+CH₂).

TABLE 10
Characterization of GALA-PSar precursor

| | DP for | DP for | DP for | DP for | Mw by GPC | |
|---|---|---|---|---|---|---|
| Code | GluOtBu^{a} (n) | Leu^{a} (e) | Ala^{a} (b) | Sar^{a} (n) | (Da)^{b} | PDI |
| C1 | 8 | 7 | 18 | 61 | 7900 | 1.122 |

^{a}Determined by NMR. Determined by GPC
C1 = NH2-Et-PGlu(OtBu)(8)-co-PAla(18)-co-PLeu(7)-b-PSar(61)

wherein the cited DP numbers are subject to a reasonable uncertainty within the range ±20%.

### 5.3 General Procedure for Deprotection Step:

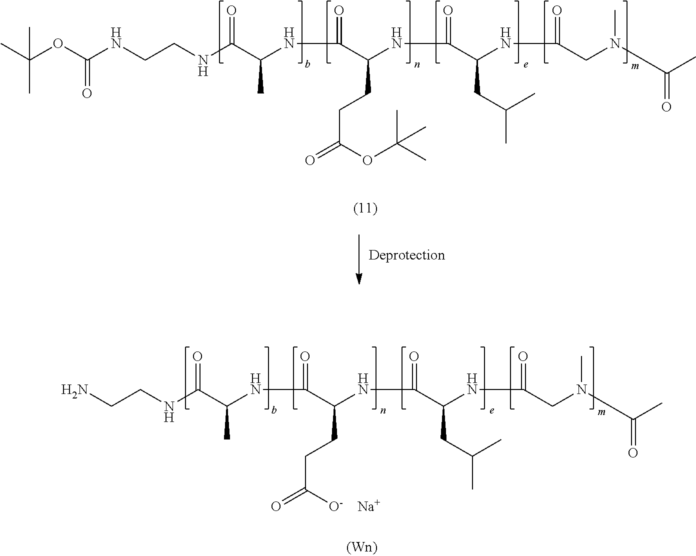

The block copolymer of Boc-NH-ethyl-R5-b-PSar was dissolved in trifluoroacetic acid at 0° C. (100 mg/mL) and the mixture was stirred at 5° C. for 2 hours. The reaction mixture was poured into diethyl ether to precipitate the product. The precipitate was isolated by centrifugation (3750 rpm, 4 min) and dried under vacuum. The isolated polymer was neutralized to pH 6.5-7 and was purified by centrifugal-assisted ultrafiltration. After filtration, the remaining aqueous polymeric solution was lyophilized to obtain the final product.

Yield: 70-80% (W1)

(D2O): δ 0.98 (d, J=14.2 Hz, CH3), 1.62 (m, CH3 Ala+CH Leu), 1.96-2.62 (m, CH2), 2.77-3.38 (m, CH3), 3.63 (brs, CH2), 4.01 (brs, CH2), 4.05-4.64 (m, CH3 Sar+CH Glu, Ala, Leu).

TABLE 11
Targeted shielding diblock copolymers according to formula (Bn)

| | | | | | Mw by | |
|---|---|---|---|---|---|---|
| | DP (n) for | DP (n) for | DP (n) for | DP (n) for | SEC | |
| Code | Glu^{a} | Ala^{a} | Leu^{a} | Sar^{a} | (Da)^{b} | PDI |
| W1 | 8 | 18 | 7 | 61 | 7628 | * |

^{a}Determined by NMR.
^{b}Estimated MW by GPC of the precursor..
* Not determined
W1 = nBu-PGlu(8)-co-PAla(18)-co-PLeu(7)-b-PSar(61)

wherein the cited DP numbers are subject to a reasonable uncertainty within the range ±20%.

### Example 6. Synthesis of Shielded Detachable Block Copolymers PSar-Succ-Detachable-GALA-Like Random Copolymer (Y)

### 6.1 General Procedure for the Polymerization and End Capping of PSar-Succ.

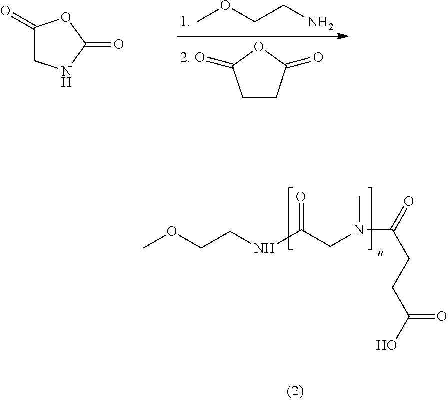

The experimental procedure is the same as described above in section 1.1B.

### 6.2 General Procedure for Shielded Detachable MeA-PSar-Succ-Cysteamine-Fmoc.

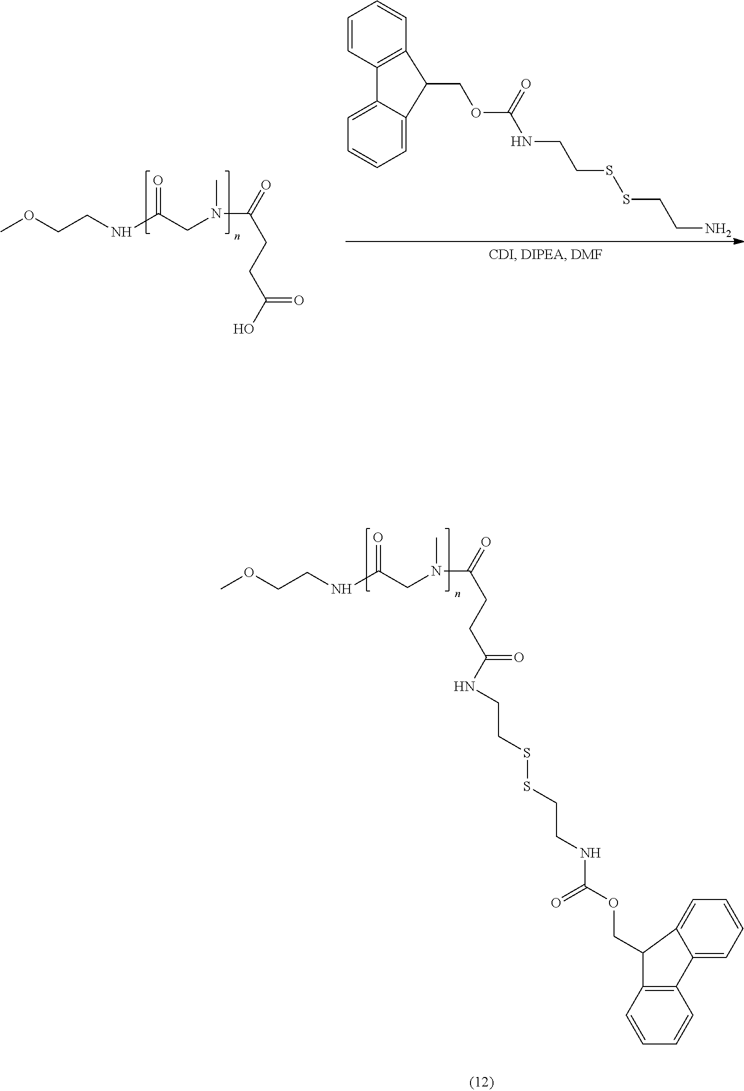

PSar-Succ (0.18 mmol, 1 g) was added to a two-necked round bottom flask fitted with a stirring bar and a stopper, then purged with 3 cycles of vacuum/N₂, and dissolved in 4 mL of DMF. Then, CDI (2.1 eq, 0.38 mmol) was added to the reaction mixture and stirred for 30 minutes at room temperature. After this time, Fmoc-cysteamine (1.2 eq, 0.21 mmol) and DIPEA (1 eq, 0.21 mmol) dissolved in 30 mL of DMF was added. The mixture was stirred at room temperature for 16 hours. The reaction mixture was poured into diethyl ether to precipitate the product. Precipitate was isolated by centrifugation (3750 rpm, 4 min) and dried under vacuum. The final product was isolated as a white solid. These system will be analyzed after the fmoc deprotection reaction.

### 6.3 General Procedure for Fmoc Deprotection:

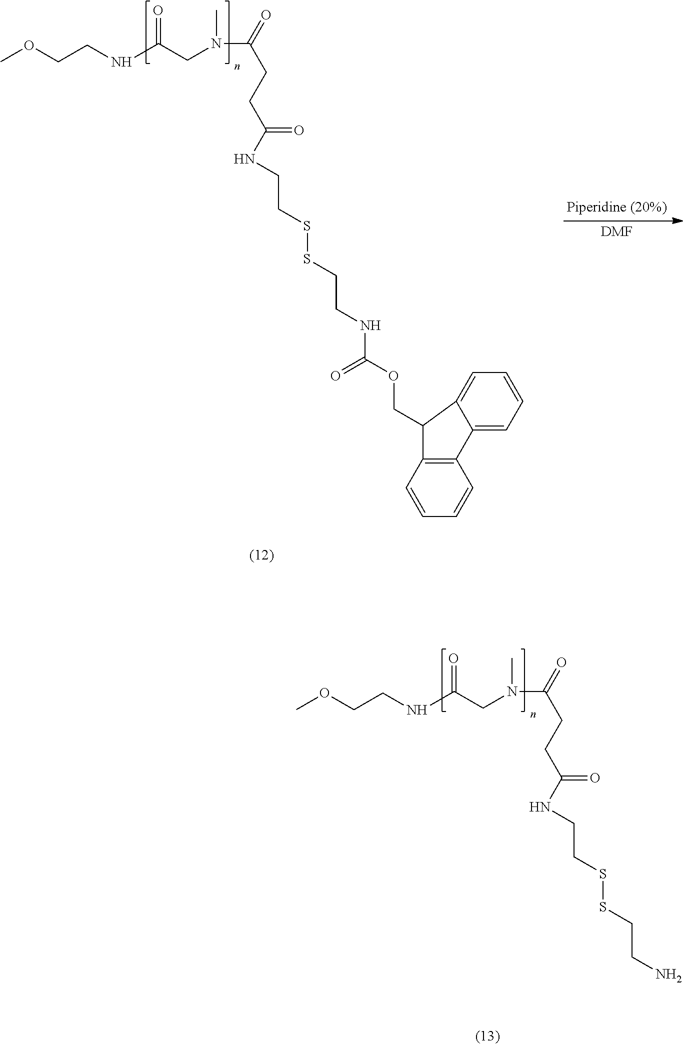

MeA-PSar-Succ-cysteamine-Fmoc (1 g) was added to a two-necked round bottom flask fitted with a stirring bar and a stopper, then purged with 3 cycles of vacuum/N₂, and dissolved in 10 mL of DMF. Then, Piperidine (2 mL) was added to the reaction mixture and stirred for 2 hours at room temperature. The reaction mixture was poured into diethyl ether to precipitate the product. Precipitate was isolated by centrifugation (3750 rpm, 4 min) and dried under vacuum. The final product was isolated as a white solid.

Yield: 95%. 1H NMR (300 MHz, TFA): δ 2.50 (m, CH₂), 2.74 (m, CH₂), 2.84-3.24 (m, CH₂), 3.38 (s, CH₃), 3.54 (m, CH₂), 4.28 (m, CH₂).

### 6.4 Procedure for the Polymerization of Copolymer PSar-Succ-Detachable-GALA-Like Random Copolymer (Y).

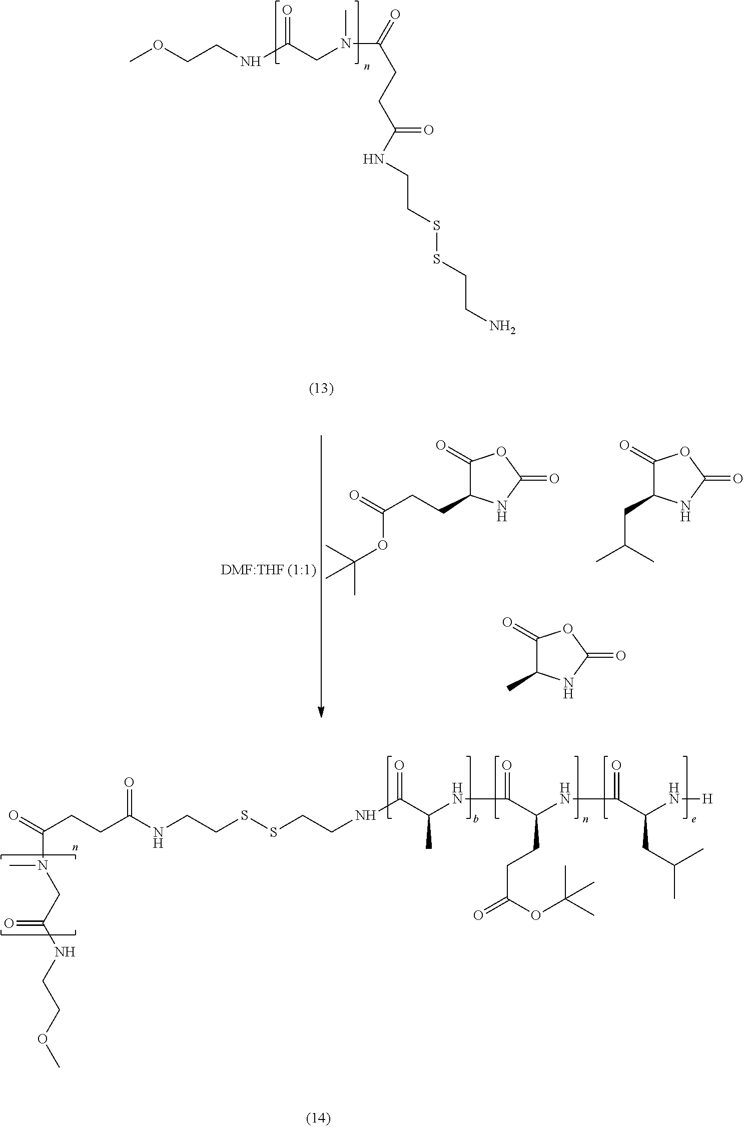

Glutamic acid t-tert butyl ester NCA, Leucine NCA and Alanine NCA were added to a Schlenk tube fitted with a stirring bar and a stopper. After 3 cycles of vacuum/N2, the mixture was dissolved in anhydrous mixture of DMF:THF (1:1). Then, the initiator (MeA-PSar-Succ-cysteamine) diluted in DMF (2 mL) and was added to the reaction mixture, which was stirred at 10° C. for 16 hours. Once NCA consumption was confirmed by IR. The reaction mixture was poured into diethyl ether to precipitate the product. The precipitate was isolated by centrifugation (3750 rpm, 4 min) and dried under vacuum. Random copolymer was isolated as a white solid.

Yield: 60-70%. 1H NMR (300 MHz, TFA): δ 1.07 (d, J=8.9 Hz, CH₃), 1.63 (brs, CH₃), 1.69-2.32 (m, CH₂), 2.78 (m, CH₂), 2.96-3.56 (m, CH₃), 3.70 (s, CH₃), 3.84 (M, CH₂), 3.96 (M, CH₂), 4.42-5.06 (m, CH₂).

TABLE 12
Characterization of PSar-succ-detachable-
Gala Random copolymer precursor

| | DP for | DP for | DP for | DP for | Mw by GPC | |
|---|---|---|---|---|---|---|
| Code | GluOtBu^{a} (n) | Leu^{a} (e) | Ala^{a} (b) | Sar^{a} (n) | (Da)^{b} | PDI |
| 14.1 | 6 | 5 | 10 | 50 | 6080 | 1.02 |

^{a}Determined by NMR. Determined by GPC
14.1 = PSar(50)-succ-detachable-[PGlu(OtBu)(6)-co-PAla(10)-co-PLeu(5)]

wherein the cited DP numbers are subject to a reasonable uncertainty within the range ±20%.

### 6.5 General Procedure for Deprotection Step:

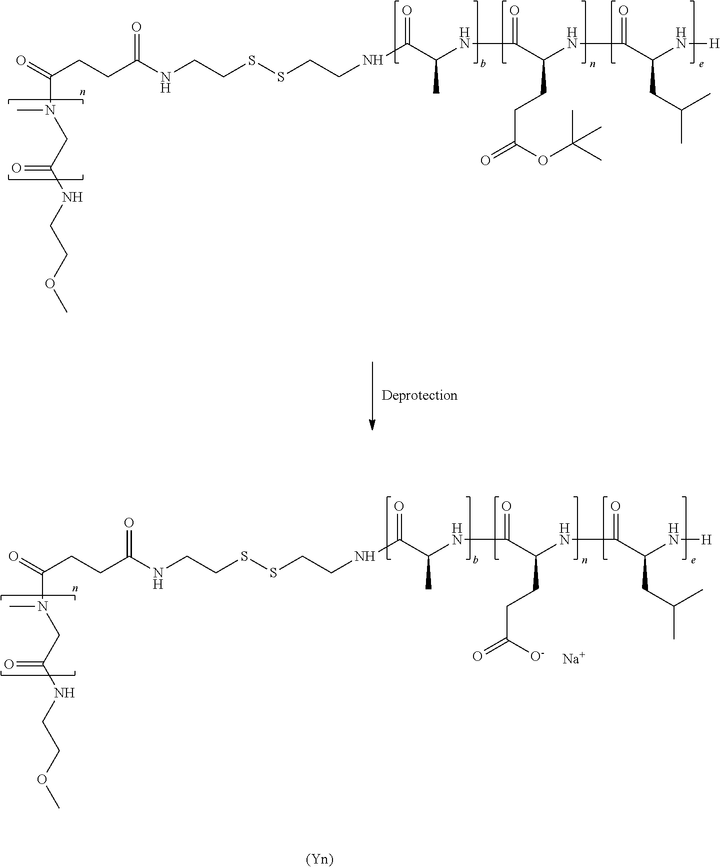

The block copolymer of PSar(50)-succ-detachable-[PGlu(OtBu)(6)-co-PAla(10)-co-PLeu(5)] was dissolved in trifluoroacetic acid at 0° C. (100 mg/mL) and the mixture was stirred at 5° C. for 2 hour. The reaction mixture was poured into diethyl ether to precipitate the product. The precipitate was isolated by centrifugation (3750 rpm, 4 min) and dried under vacuum. The isolated polymer was neutralized to pH 6.5-7 and was purified by centrifugal-assisted ultrafiltration. After filtration, the remaining aqueous polymeric solution was lyophilized to obtain the final product.

Yield: 70-80% (Y1) 1H NMR (300 MHz (D2O): δ 0.98 (d, J=20.6 Hz, CH₃), 1.53 (brs, CH3 Ala+CH Leu), 1.65-2.44 (m, CH₂), 2.69 (m, CH₂), 3.60 (s, CH₃), 3.75 (m, CH₂), 3.86 (m, CH₂), 4.05-4.64 (m, CH₃ Sar+CH Glu, Ala, Leu).

TABLE 13
Targeted shielding diblock copolymers according to formula (Yn)

| | | | | | Mw by | |
|---|---|---|---|---|---|---|
| | DP (n) for | DP (n) for | DP (n) for | DP (n) for | SEC | |
| Code | Glu^{a} | Ala^{a} | Leu^{a} | Sar^{a} | (Da)^{b} | PDI |
| Y1 | 6 | 10 | 5 | 50 | 6045 | * |

^{a}Determined by NMR.
^{b}Estimated MW by GPC of the precursor.
* Not determined
Y1 = PSar(50)-succ-detachable-[PGluONa(6)-co-PAla(10)-co-PLeu(5)]

wherein the cited DP numbers are subject to a reasonable uncertainty within the range ±20%.

### Example 7. Synthesis of Comparative Example where (R5) Contains Only One of the Disclosed Groups According to the Present Invention: PGluONa-Co-PLeu-b-PSar Copolymers (E)

The following example has been synthesized for comparative purposes following the procedure described in: Tian, C., Ling, J., & Shen, Y. qing. (2015). Self-assembly and pH-responsive properties of poly(L-glutamic acid-r-L-leucine) and poly(L-glutamic acid-r-L-leucine)-b-polysarcosine. Chinese Journal of Polymer Science (English Edition), 33(8), 1186-1195. https://doi.org/10.1007/s10118-015-1669-0. The following product was further used to stabilize different polyplexes, either formed by N1 or PEI, with negative results (See examples 10.6 and 10.9)

### 7.1 General Procedure for the Polymerization of PSar-b-PGluOtBu

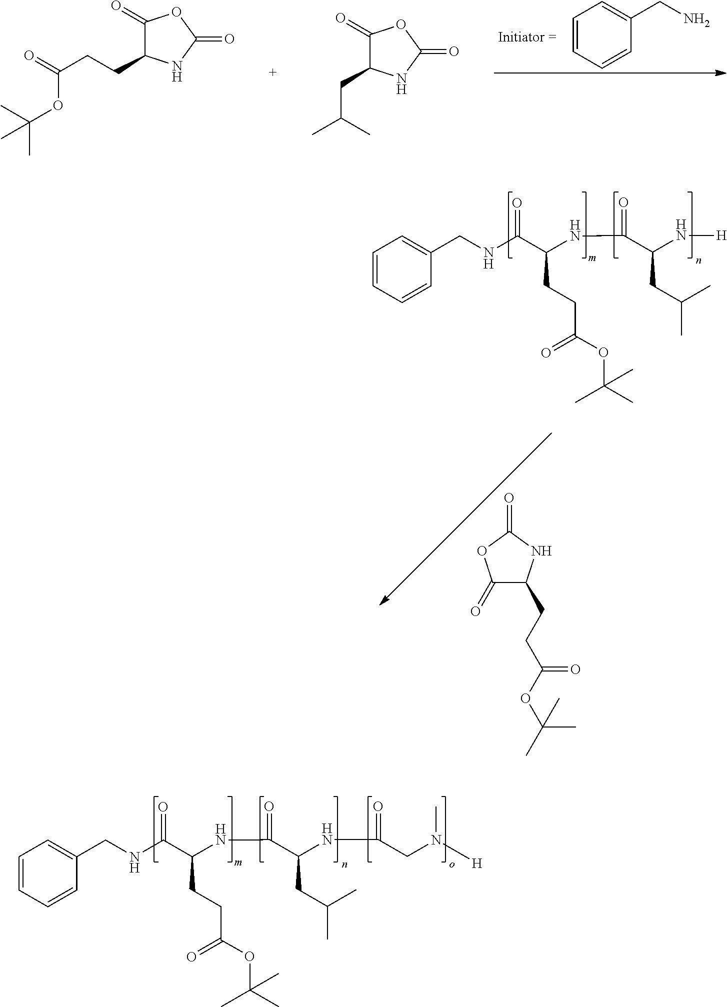

Glutamic acid t-tert butyl ester NCA and L-Leucine NCA were added to a Schlenk tube fitted with a stirring bar and a stopper. After 3 cycles of vacuum/N₂, the mixture was dissolved in anhydrous DMF (150 mg/mL). Then, the initiator (benzylamine) diluted in DMF (2 mL) and was added to the reaction mixture, which was stirred at 10° C. for 16 hours. Once NCA consumption was confirmed by IR the Sarcosine-N-carboxyanhydride (Sar-NCA) was added to the reaction mixture dissolved in anhydrous DMF. The mixture was stirred at 10° C. for 16 hours. Upon completion the reaction mixture became clear and full conversion of the monomer could be detected by IR. The reaction mixture was poured into diethyl ether to precipitate the product. The precipitate was isolated by centrifugation (3750 rpm, 4 min) and dried under vacuum. Block copolymer was isolated as a white solid.

Yield: 70-90%. The polymerization product was not characterized by NMR since it is only soluble in the deprotection solvent.

### 7.2 General Procedure for Deprotection Step:

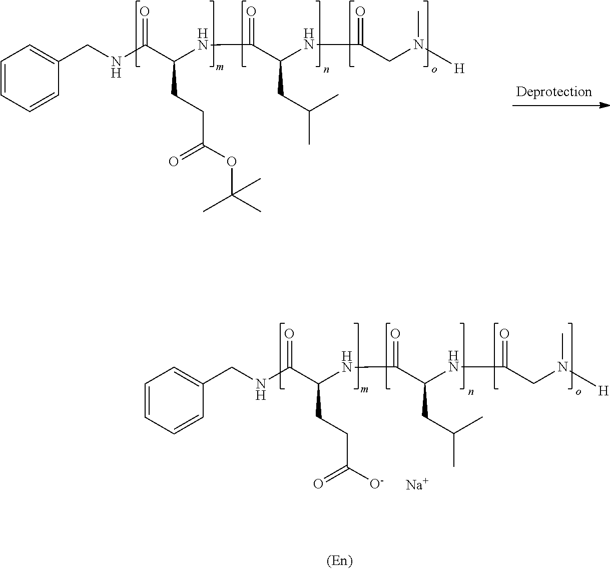

The block copolymer of Bn-PSar-b-PGluOtBu was dissolved in trifluoroacetic acid at 0° C. (100 mg/mL) and the mixture was stirred at 5° C. for 1 hour. The reaction mixture was poured into diethyl ether to precipitate the product. The precipitate was isolated by centrifugation (3750 rpm, 4 min) and dried under vacuum. The block copolymer was isolated as a white solid.

Yield: 95%.

[V1-V2]1H NMR (D2O): δ 0.93 (d, J=27.0 Hz, CH₃), 1.62 (brs, CH₂), 1.73 (brs, CH), 1.82-2.45 (m, CH₂), 2.78-3.17 (m, CH₃), 4.00-4.63 (m, CH₂ PSar+CH GluONa), 7.38 (m, aryl CH).

TABLE 14
Comparartive example of block copolymers where
(R5) contains only one aminoacid (En)

| | DP (n) | DP (n) | DP (n) | Mw by SEC | |
|---|---|---|---|---|---|
| Code | for Glu^{a} | for Leu^{a} | for Sar^{a} | (Da)^{b} | PDI |
| E1 | 32 | 7 | 45 | 8929 | * |

^{a}Determined by NMR.
^{b}Estimated MW by GPC of the precursor.
*Not determined
E1 = Bnz-[PGluONa(32)-co-Pleu(7)]-b-Psar(45) (E1)

Wherein the cited DP numbers are subject to a reasonable uncertainty within the range ±20%.

### Example 8. Synthesis and Description of Polycationic Carriers

In order to proof the stabilizing ability and the enhanced transfection features imbued to the polyplexes by the shielding polymers, the universal benchmark jetPEI was used (Polyplus-transfection S.A, Illkirch, France) (Ref Polyplus: 101-10N). JetPEI® is a powerful reagent that ensures robust, effective and reproducible DNA transfection into mammalian cells with low toxicity. jetPEI® is mainly composed of a linear polyethylenimine manufactured at Polyplus-transfection. jetPEI® is provided as a 7.5 mM solution in sterile and apyrogenic water (expressed as concentration of nitrogen residues). Also, to proof stabilization of very different architectural and compositional alternative polycations, different polycationic NVVs based on Star-shaped polyaminoacids were synthesized and its polyplexes were also stabilized and assayed. the following examples describe the preparation of the afore mentioned polycationic compounds used to complexate and vehiculize the genetic material.

### 8.1 Preparation of Compound N1

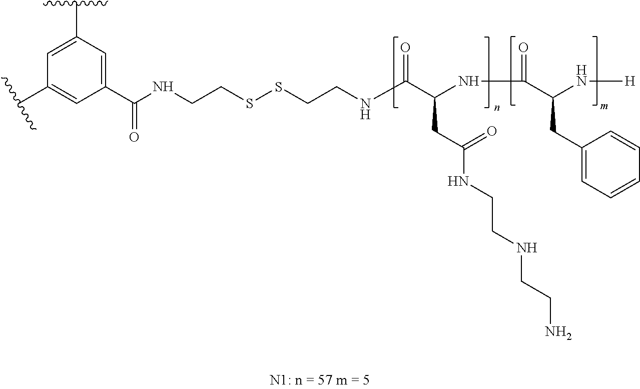

In general terms, to synthesize compounds of formula (N1) according to the present disclosure, first, the 3-arm star initiator was obtained within 2-3 steps. Such initiator was used then to polymerize y-benzyl L-Aspartate-NCA and L-Phenylalanine NCA, to yield the star random copolymer benzyl protected (St-PAsp(Bz)-co-PPhe). The benzyl groups were removed by an aminolysis reaction to yield the corresponding Star-PAsp-Oligoamine-co-PPhe.

Scheme 1 shows a particular example of polymerization and aminolysis steps:

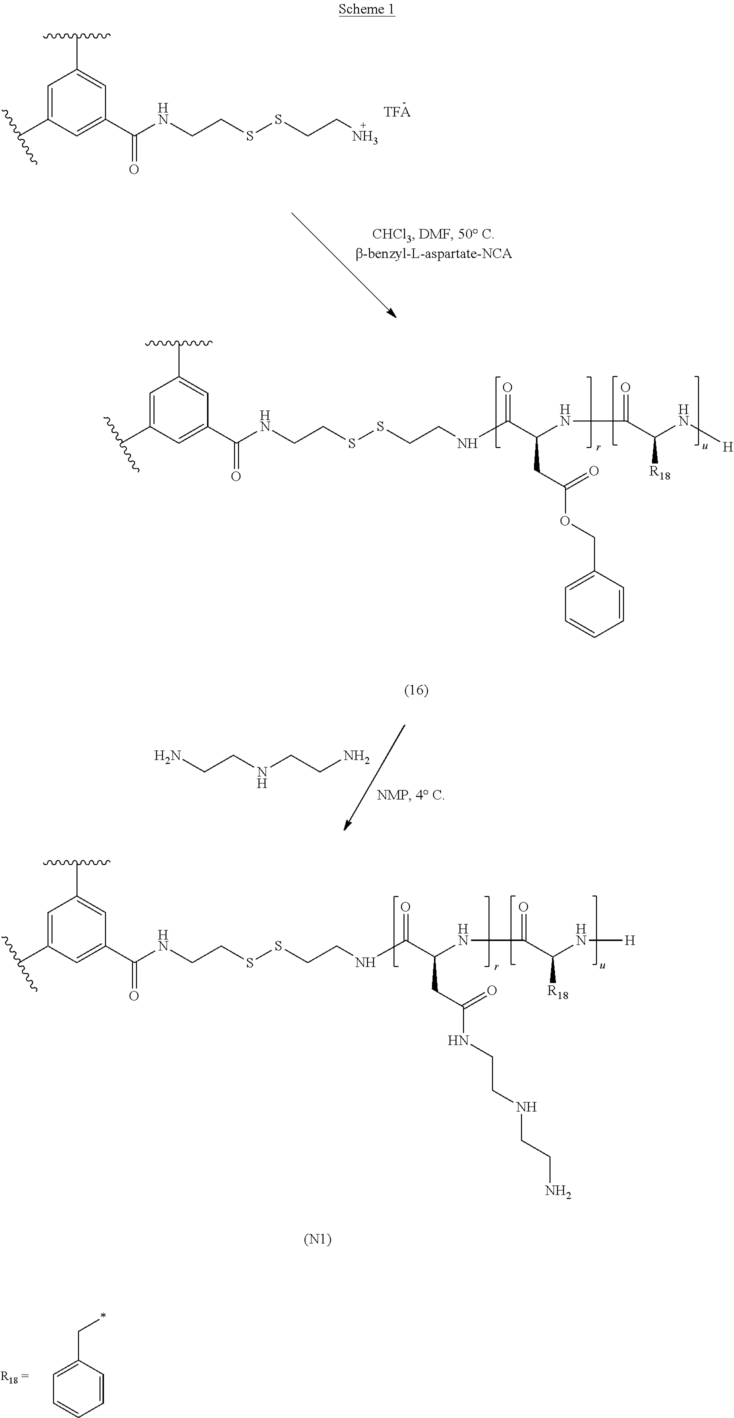

### 8.1.A Synthesis of 3-Arm Star Initiators

The synthetic routes towards 3-arm star initiators is described below.

### Example 8.1.A: Trifluoroacetic acid salt of N,N,N-tris(2-((2-Aminoethyl) disulfanyl)ethyl)benzene-1,3,5-tricarboxamide (St-S—S-Initiator) (15)

Trifluoroacetic acid salt of N,N,N-tris(2-((2-Aminoethyl)disulfanyl)ethyl)benzene-1,3,5-tricarboxamide (St-S—S-initiator) (17) was synthesized following the general procedure disclosed Scheme 2.

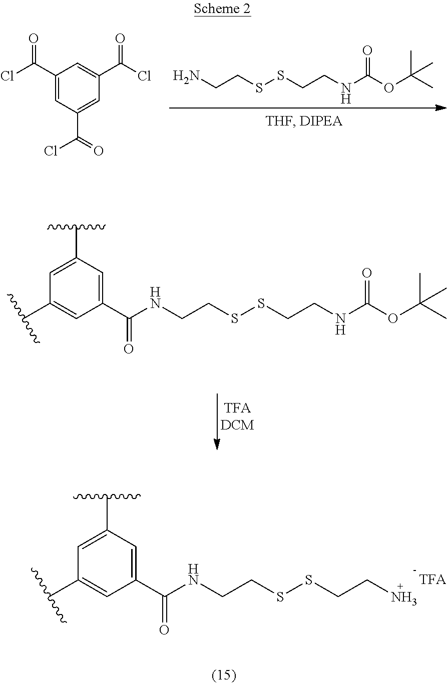 [SMILES: O=C(Cl)c1cc(C(=O)Cl)cc(C(=O)Cl)c1 ; CC(C)(C)OC(=O)NCCSSCCN ; *c1cc(*)cc(C(=O)NCCSSCCNC(=O)OC(C)(C)C)c1 ; *c1cc(*)cc(C(=O)NCCSSCCN)c1]

The synthesis of the trimeric amine initiator starts with a coupling reaction followed by amine deprotection.

### Step (a) Synthesis of tri-tert-butyl ((((benzenetricarbonyltris-(azanediyl))tris(ethane-2,1-diyl))tris(disulfanediyl))tris(ethane-2,1-diyl))-tricarbamate

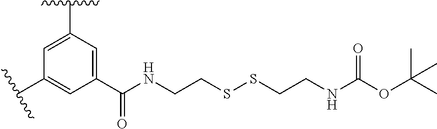 [SMILES: *c1cc(*)cc(C(=O)NCCSSCCNC(=O)OC(C)(C)C)c1]

N-(tert-Butyloxycarbonyl)cystamine (7.99, 27 mmol, 3.3 eq) was weighed into a flame-dried two-neck round-bottom flask and was dissolved in 56 mL anhydrous THF. Freshly distilled DIPEA (4.75 mL, 27 mmol, 3.3 eq) was added and stirred for 15 min at room temperature. 1,3,5-Benzenetricarbonyl trichloride (2.25 g, 8.3 mmol, 1 eq) was weighed into a flame-dried two-neck round-bottom flask and was dissolved in 28 mL of anhydrous THF. The trichloride solution was slowly added to the N-(tert-butyloxycarbonyl)cystamine mixture via syringe. The progress of reaction was monitored by thin-layer chromatography (TLC). After 4 h, the solvent was evaporated in vacuo and the residue was dissolved in ethyl acetate. The organic layer was sequentially washed with Milli-Q water, 1M hydrochloric acid and saturated sodium bicarbonate solution. The organic phase was dried over magnesium sulfate anhydrous and concentrated in vacuo afforded tri-tert-butyl ((((benzenetricarbonyltris-(azanediyl))tris(ethane-2,1-diyl))tris-(disulfanediyl))tris(ethane-2,1-diyl))-tricarbamate as a white foam (7.5 g, η=98%).

¹H NMR (CDCl₃): δ=1.39 (brs, 27H, —C(CH₃)₃), 2.84 (t, J=6.26 Hz, 6H, CH₂), 2.96 (t, J=6.84 Hz, 6H. CH₂), 3.46 (m, 6H, CH₂), 3.79 (m, 6H, CH₂), 5.18 (brs, 3H, —NHBoc), 7.39 (brs, 3H, aryl CH).

### Step (b): Synthesis of trifluoroacetic acid salt of N,N,N-tris(2-((2-aminoethyl)disulfanyl)ethyl)benzene-1,3,5-tricarboxamide(St-S—S-Initiator) (15)

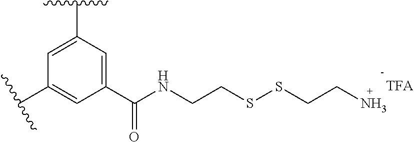 [SMILES: *c1cc(*)cc(C(=O)NCCSSCCN)c1]

7.5 g (8.19 mmol) of the initiator (15) was dissolved in anhydrous dichlorometane (180 mL) and 90 mL of TFA were added. The reaction was stirred under nitrogen atmosphere for 60 min and the completion of the reaction was monitored by TLC. The solvents were evaporated under vacuum. The TFA salt of the initiator (15) (7 g, 7.31 mmol) was obtained in quantitative yield and dried under vacuum.

1H NMR (D2O): δ=2.86 (m, 12H), 3.25 (t, J=6.49 Hz, 8H), 3.60 (t, J=6.85 Hz, 8H), 8.02 (brs, 3H, aryl CH).

### Example 8.1.B. Synthesis of Star-PAsp(Bz) (16) Copolymers Comprising a Hydrophobic Fragment

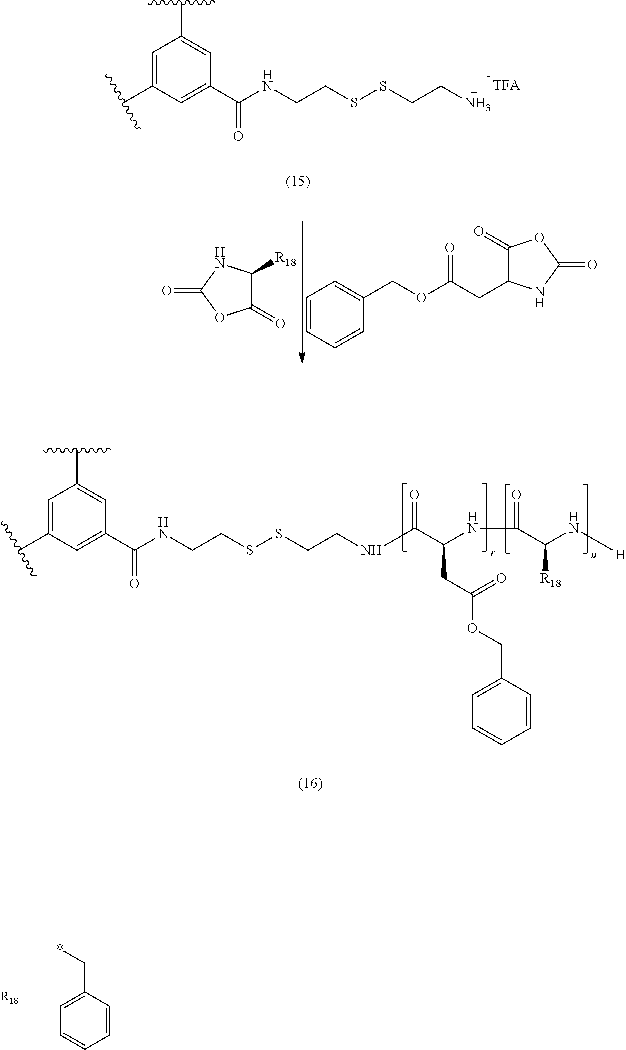

To synthesize the copolymers with hydrophobic residues, the polymerization was carried out via ring opening polymerization mechanism using trifluoroacetic acid salt of N,N,N-tris(2-((2-aminoethyl)disulfanyl)ethyl)-benzene-1,3,5-tricarboxamide as initiator.

### General Procedure for Synthesized St-S—S-PAsp(Bz)(45)-Co-PLeu(5)

Bi-benzyl-L-aspartate-N-carboxy anhydride (3.5 g, 14.15 mmol) and L-Phenylalanine-N-carboxy anhydride (1.57 mmol) was added to a Schlenk tube fitted with a stirrer bar and a stopper, and purged with 3 cycles of vacuum/N₂, and dissolved in a mixture of anhydrous chloroform (100 mL) and DMF (6 mL). Then, the star initiator was dissolved in DMF (4 mL) and was added to the reaction mixture. The mixture was stirred at 50° C. for 16 hours. Upon completion, the reaction mixture became clear and full conversion of the monomer could be detected by IR. The reaction mixture was poured into diethyl ether to precipitate the product. The precipitate was isolated by centrifugation (3750 rpm, 4 min) and dried under vacuum. Copolymer was isolated as a white solid.

Yield: 70-80% 1H NMR (TFA): 5=2.99 (s, 2H, CH), 3.94 (brs, 1H, CH), 4.93 (s, 1H, CH), 5.15 (m, 2H, benzyl CH2), 7.20 (s, 5H, aryl CH), 8.42 (s, aryl CH).

The introduction of ratios of the repeating unit were adjusted changing the mixing ratios of the corresponding monomer units to be allowed to react. In this precursor, the hydrophobic residue matches by 1H-NMR with the protective group of the poly-aspartic. This system will be analyzed after the aminolysis reaction (next example 5.1.C).

### Example 8.1.C Synthesis of Amphiphilic Polyaspartamide Derivatives St-S—S-PAspDET-Co—PR18 (X)

As shown in the synthetic route below (7) polyamino acids were prepared by the simultaneous aminolysis reaction of PBLA with DET.

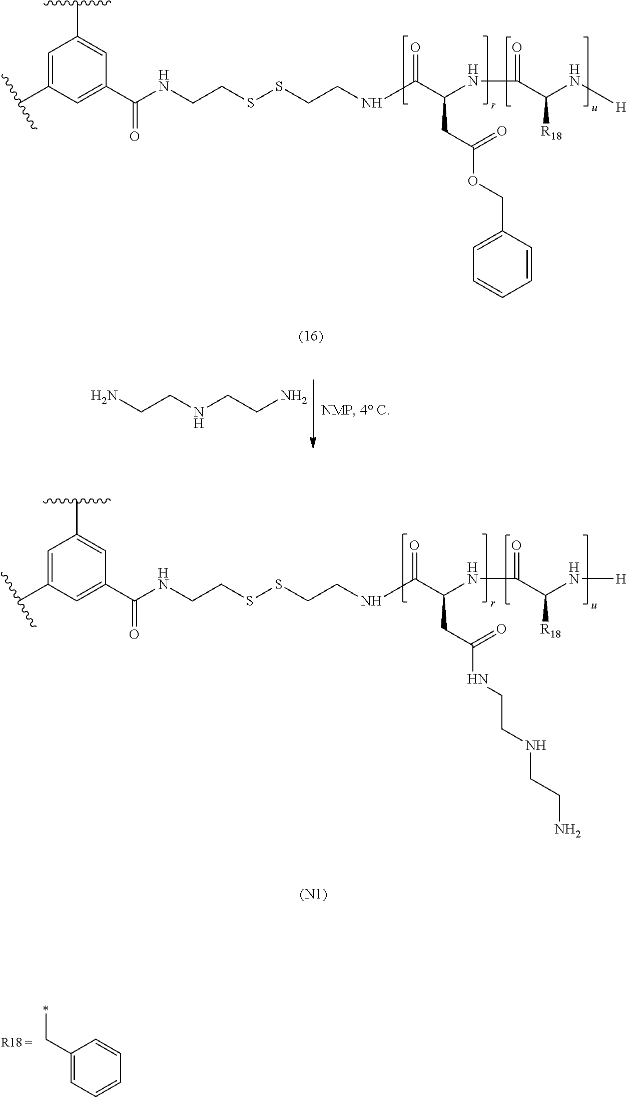

As an example, herein we describe the synthetic method in which R₁₈ represents a phenylalanine group. Copolymer of St-S—SPAsp(Bz)45-co-PPhe(5) (500 mg of copolymer, 470 mg of PBLA, DP:45) was dissolved in NMP (10 mL) and cooled to 4° C. The resultant copolymer solution was added dropwise to the mixture of DET (12 mL, 50 eq. vs unit of PAsp(Bz)) and the solution was stirred for 4 h at 4° C. under nitrogen atmosphere. After this time, the reaction mixture was added dropwise into cold HCl 6 M for neutralization (pH 3.5). The polymer product was purified by centrifugal-assisted ultrafiltration. After filtration, the remaining aqueous polymeric solution was lyophilized to obtain the final product.

Yield: 70-80%. ¹H NMR (D₂O) [R₁₈=Phe side chain]: 5=2.91 (brs, 2H, CH2), 3.84-3.18 (m, 2H, CH2), 7.34 (brs, 5H, aryl CH of Phe), 8.33 (s, aryl CH).

Table 15 refers to amphiphilic copolymer St-S—S-PAspDET-co-R₁₈ according to formula (N1).

TABLE 15
Characterization data of N1 polymer

| | | Ratio^{a} | Mn^{b} | |
|---|---|---|---|---|
| Compound | R18 | PAsp:R18 | (Da) | Ð |
| N1 | Phe | 57:5 | 13098 | 1.282 |

^{a}Determined by NMR.
^{b}Determined by SEC.
Mn and DP refer to number average molar mass and degree of polymerization respectively.

### 8.2 Preparation of Compound N2

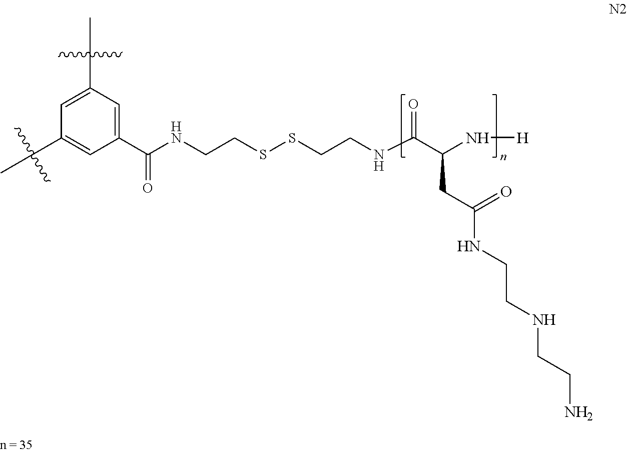

### Example 8.2.A: St-Poly(p-Benzyl-L-Aspartate) (Star-PAsp(Bz)) (Pn)

General procedure for the polymerization of St-PAsp(Bz)(6) is as follows:

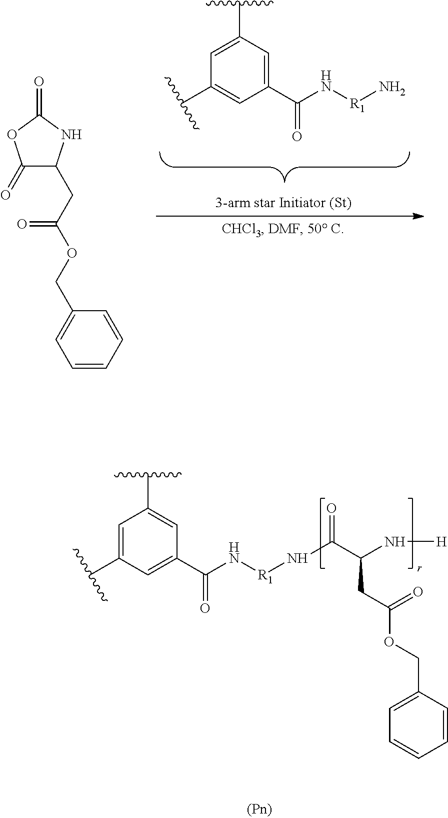

The synthesis of the compound N2 is very similar as described in the previous example and starts with the same indicator previously described. p-benzyl-L-aspartate-NCA (5 g, 2 mmol) was added to a Schlenk tube fitted with a stirrer bar, a stopper and purged with 3 cycles of vacuum/N₂, and dissolved in a mixture of anhydrous chloroform (100 mL) and DMF (6 mL). Then, the star initiator (St) was dissolved in DMF (4 mL) and was added to the reaction mixture. The mixture was stirred at 50° C. for 16 hours. Upon completion, the reaction mixture became clear and full conversion of the monomer could be detected by IR. The reaction mixture was poured into diethyl ether to precipitate the product. The precipitate was isolated by centrifugation (3750 rpm, 4 min) and dried under vacuum. St-Poly(βbenzyl-L-aspartate) (Star-PAsp(Bz)) (Pn) was isolated as a white solid.

Yield: 70-90% ¹H NMR (TFA): 5=2.92 (m, 2H, CH₂), 4.85 (s, 1H, CH), 5.05 (m, 2H, benzyl CH₂), 7.13 (s, 5H, aryl CH), 8.38 (s, aryl CH).

TABLE 16
Characterization data of precursor compound P1

| | | | Mn^{a} | |
|---|---|---|---|---|
| Compound | R1 | DP_{PAsp(Bz) theoretical} | (kDa) | DP_{PAsp(Bz)}^{a} |
| P1 | —CH₂CH₂SSCH₂CH₂— | 50 | 10.5 | 47 |

^{a}Estimated by NMR.
Mn and DP refer to number average molar mass and degree of polymerization respectively

### Example 8.2.B Aminolysis Reaction of Poly(p-Benzyl-L-Aspartate) (6) to Afford St-PAsp-DET (Nn)

General procedure for the aminolysis reaction to generate polycationic homopolymer PaspDET regardless of the nature of the initiator used in the polymerization step:

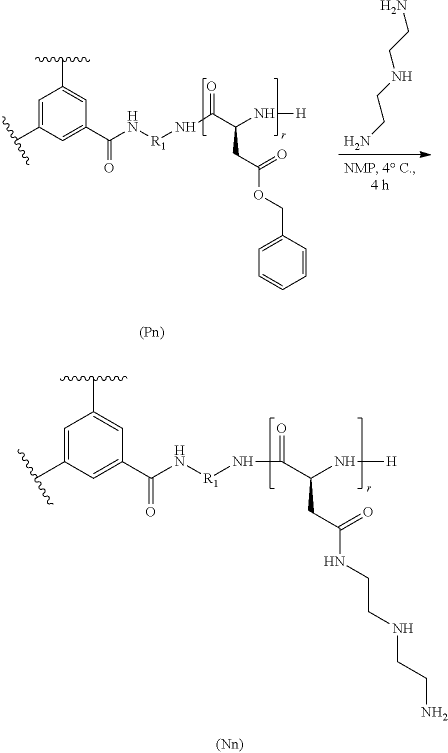

St-PAsp(Bz)(6) (DP=50, 750 mg) was dissolved in NMP (15 mL) and cooled to 4° C. This solution was added dropwise to the DET (50 eq DET vs unit of Asp, 19 mL) cooled at 4° C. and the mixture was stirred for 4 hours at the same temperature. After this time, the reaction mixture was added dropwise into cold HCl 6M for neutralization (pH 3.5). The polymer product was purified by centrifugal-assisted ultrafiltration. After filtration through 0.22 um PES filter, the remaining aqueous polymeric solution was lyophilized to obtain the final product (370 mg, q=50%).

¹H NMR (D₂O): δ 2.93 (brs, 2H, CH₂), 3.12-3.85 (m, 2H, CH₂), 8.33 (s, 3H, aryl CH).

TABLE 17
Characterization of compound N2 St-PAsp-DET.

| | | | Mn^{a} | Mn^{b} | | | |
|---|---|---|---|---|---|---|---|
| Compound | R1 | DP_{AspDET theo} | (kDa) | (kDa) | DP^{a} | DP^{b} | Ð |
| N2 | —CH₂CH₂SSCH₂CH₂— | 35 | 11 | 6.9 | 51 | 31 | 1.305 |

^{a}Determined by NMR.
^{b}Determined by SEC.
Mn and DP refer to number average molar mass and degree of polymerization respectively.
Ð represent polydispersity as determined by SEC-MALS software analysis.

### Example 9. Polyplex Formulation

Polyplex formulations are named as “PXn_ratio1_Shielding polymer_ratio2^{nuc}”, wherein “n” corresponds to the polycationic compound nomenclature as given above, which is used to form the polyplex; wherein “ratio1” refers to the N/P ratio of cationic polymer vs genetic material “shielding polymer” refers to the polyanionic shielding diblock copolymer and “ratio2” refers to the +/− ratio of the cationic polymer vs shielding (anionic) polymer; and wherein “nuc” refers to the type of nucleic acid: pDNA, cIDNA or mRNA.

In the following examples, a pDNA (purchased from PlasmidFactory, with reference PF461 (pCMV-luc)), containing 6233 bp expressing luciferase) and a cIDNA according to SEQ ID NO. 1 which was obtained according to standard molecular biology methods, such as the one disclosed in Heinrich, M. et al. “Linear closed mini DNA generated by the prokaryotic cleaving-joining enzyme TeIN is functional in mammalian cells”, J Mol Med, 2002, vol. 80, pp. 648-654. The mRNA was purchased from Trilink, with reference L-1201-1000 CleanCap Fluc mRNA (5moU) expressing luciferase as reporter gene.

The sequence of the cIDNA according to SEQ ID NO. 1, in the examples, is that of Table 18.

TABLE 18
Sequence of the clDNA (SEQ ID NO. 1)
SEQ ID NO: 1; linear closed DNA (clDNA) synthetic construct (artificial)
with 5580 nucleotides.

| | |
|---|---|
| gcgtataatg gactattgtg tgctgatagg tctcgccctg atttaaatcg tcgaccgtta | 60 |
| cataacttac ggtaaatggc ccgcctggct gaccgcccaa cgacccccgc ccattgacgt | 120 |
| caataatgac gtatgttccc atagtaacgc caatagggac tttccattga cgtcaatggg | 180 |
| tggagtattt acggtaaact gcccacttgg cagtacatca agtgtatcat atgccaagta | 240 |
| cgccccctat tgacgtcaat gacggtaaat ggcccgcctg gcattatgcc cagtacatga | 300 |
| ccttatggga ctttcctact tggcagtaca tctacgtatt agtcatcgct attaccatgg | 360 |
| tgatgcggtt ttggcagtac atcaatgggc gtggatagcg gtttgactca cggggatttc | 420 |
| caagtctcca ccccattgac gtcaatggga gtttgttttg gcaccaaaat caacgggact | 480 |
| ttccaaaatg tcgtaacaac tccgccccat tgacgcaaat gggcggtagg cgtgtacggt | 540 |
| gggaggtcta tataagcaga gctcgtttag tgaaccgtca gatcgcctgg agacgccatc | 600 |
| cacgctgttt tgacctccat agaagacacc gggaccgatc cagcctccgg actctagagg | 660 |
| atccggtact cgaggaactg aaaaaccaga aagttaactg gtaagtttag tctttttgtc | 720 |
| ttttatttca ggtcccggat ccggtggtgg tgcaaatcaa agaactgctc ctcagtggat | 780 |
| gttgccttta cttctaggcc tgtacggaag tgttacttct gctctaaaag ctgcggaatt | 840 |
| gtacccgcgg cccgggatcc accggtcgcc accatggaag acgccaaaaa cataaagaaa | 900 |
| ggcccggcgc cattctatcc tctagaggat ggaaccgctg gagagcaact gcataaggct | 960 |
| atgaagagat acgccctggt tcctggaaca attgctttta cagatgcaca tatcgaggtg | 1020 |
| aacatcacgt acgcggaata cttcgaaatg tccgttcggt tggcagaagc tatgaaacga | 1080 |
| tatgggctga atacaaatca cagaatcgtc gtatgcagtg aaaactctct tcaattcttt | 1140 |
| atgccggtgt tgggcgcgtt atttatcgga gttgcagttg cgcccgcgaa cgacatttat | 1200 |
| aatgaacgtg aattgctcaa cagtatgaac atttcgcagc ctaccgtagt gtttgtttcc | 1260 |
| aaaaaggggt tgcaaaaaat tttgaacgtg caaaaaaaat taccaataat ccagaaaatt | 1320 |
| attatcatgg attctaaaac ggattaccag ggatttcagt cgatgtacac gttcgtcaca | 1380 |
| tctcatctac ctcccggttt taatgaatac gattttgtac cagagtcctt tgatcgtgac | 1440 |
| aaaacaattg cactgataat gaattcctct ggatctactg ggttacctaa gggtgtggcc | 1500 |
| cttccgcata gaactgcctg cgtcagattc tcgcatgcca gagatcctat ttttggcaat | 1560 |
| caaatcattc cggatactgc gattttaagt gttgttccat tccatcacgg ttttggaatg | 1620 |
| tttactacac tcggatattt gatatgtgga tttcgagtcg tcttaatgta tagatttgaa | 1680 |
| gaagagctgt ttttacgatc ccttcaggat tacaaaattc aaagtgcgtt gctagtacca | 1740 |
| accctatttt cattcttcgc caaaagcact ctgattgaca aatacgattt atctaattta | 1800 |
| cacgaaattg cttctggggg cgcacctctt tcgaaagaag tcggggaagc ggttgcaaaa | 1860 |
| cgcttccatc ttccagggat acgacaagga tatgggctca ctgagactac atcagctatt | 1920 |
| ctgattacac ccgaggggga tgataaaccg ggcgcggtcg gtaaagttgt tccatttttt | 1980 |
| gaagcgaagg ttgtggatct ggataccggg aaaacgctgg gcgttaatca gagaggcgaa | 2040 |
| ttatgtgtca gaggacctat gattatgtcc ggttatgtaa acaatccgga agcgaccaac | 2100 |
| gccttgattg acaaggatgg atggctacat tctggagaca tagcttactg ggacgaagac | 2160 |
| gaacacttct tcatagttga ccgcttgaag tctttaatta aatacaaagg atatcaggtg | 2220 |
| gcccccgctg aattggaatc gatattgtta caacacccca acatcttcga cgcgggcgtg | 2280 |
| gcaggtcttc ccgacgatga cgccggtgaa cttcccgccg ccgttgttgt tttggagcac | 2340 |
| ggaaagacga tgacggaaaa agagatcgtg gattacgtcg ccagtcaagt aacaaccgcg | 2400 |
| aaaaagttgc gcggaggagt tgtgtttgtg gacgaagtac cgaaaggtct taccggaaaa | 2460 |
| ctcgacgcaa gaaaaatcag agagatcctc ataaaggcca agaagggcgg aaagtccaaa | 2520 |
| ttgtaaagcg gccgcgggga tccagacatg ataagataca ttgatgagtt tggacaaacc | 2580 |
| acaactagaa tgcagtgaaa aaaatgcttt atttgtgaaa tttgtgatgc tattgcttta | 2640 |
| tttgtaacca ttataagctg caataaacaa gttaacaaca acaattgcat tcattttatg | 2700 |
| tttcaggttc agggggaggt gtgggaggtt ttttagtcga ccatttaaat cggtctcgag | 2760 |
| ggtatcagca cacaattgcc cattatacgc gcgtataatg ggcaattgtg tgctgatacc | 2820 |
| ctcgagaccg atttaaatgg tcgactaaaa aacctcccac acctccccct gaacctgaaa | 2880 |
| cataaaatga atgcaattgt tgttgttaac ttgtttattg cagcttataa tggttacaaa | 2940 |
| taaagcaata gcatcacaaa tttcacaaat aaagcatttt tttcactgca ttctagttgt | 3000 |
| ggtttgtcca aactcatcaa tgtatcttat catgtctgga tccccgcggc cgctttacaa | 3060 |
| tttggacttt ccgcccttct tggcctttat gaggatctct ctgatttttc ttgcgtcgag | 3120 |
| ttttccggta agacctttcg gtacttcgtc cacaaacaca actcctccgc gcaacttttt | 3180 |
| cgcggttgtt acttgactgg cgacgtaatc cacgatctct ttttccgtca tcgtctttcc | 3240 |
| gtgctccaaa acaacaacgg cggcgggaag ttcaccggcg tcatcgtcgg gaagacctgc | 3300 |
| cacgcccgcg tcgaagatgt tggggtgttg taacaatatc gattccaatt cagcgggggc | 3360 |
| cacctgatat cctttgtatt taattaaaga cttcaagcgg tcaactatga agaagtgttc | 3420 |
| gtcttcgtcc cagtaagcta tgtctccaga atgtagccat ccatccttgt caatcaaggc | 3480 |
| gttggtcgct tccggattgt ttacataacc ggacataatc ataggtcctc tgacacataa | 3540 |
| ttcgcctctc tgattaacgc ccagcgtttt cccggtatcc agatccacaa ccttcgcttc | 3600 |
| aaaaaatgga acaactttac cgaccgcgcc cggtttatca tccccctcgg gtgtaatcag | 3660 |
| aatagctgat gtagtctcag tgagcccata tccttgtcgt atccctggaa gatggaagcg | 3720 |
| ttttgcaacc gcttccccga cttctttcga aagaggtgcg cccccagaag caatttcgtg | 3780 |
| taaattagat aaatcgtatt tgtcaatcag agtgcttttg gcgaagaatg aaaatagggt | 3840 |
| tggtactagc aacgcacttt gaattttgta atcctgaagg gatcgtaaaa acagctcttc | 3900 |
| ttcaaatcta tacattaaga cgactcgaaa tccacatatc aaatatccga gtgtagtaaa | 3960 |
| cattccaaaa ccgtgatgga atggaacaac acttaaaatc gcagtatccg gaatgatttg | 4020 |
| attgccaaaa ataggatctc tggcatgcga gaatctgacg caggcagttc tatgcggaag | 4080 |
| ggccacaccc ttaggtaacc cagtagatcc agaggaattc attatcagtg caattgtttt | 4140 |
| gtcacgatca aaggactctg gtacaaaatc gtattcatta aaaccgggag gtagatgaga | 4200 |
| tgtgacgaac gtgtacatcg actgaaatcc ctggtaatcc gttttagaat ccatgataat | 4260 |
| aattttctgg attattggta attttttttg cacgttcaaa attttttgca accccttttt | 4320 |
| ggaaacaaac actacggtag gctgcgaaat gttcatactg ttgagcaatt cacgttcatt | 4380 |
| ataaatgtcg ttcgcgggcg caactgcaac tccgataaat aacgcgccca acaccggcat | 4440 |
| aaagaattga agagagtttt cactgcatac gacgattctg tgatttgtat tcagcccata | 4500 |
| tcgtttcata gcttctgcca accgaacgga catttcgaag tattccgcgt acgtgatgtt | 4560 |
| cacctcgata tgtgcatctg taaaagcaat tgttccagga accagggcgt atctcttcat | 4620 |
| agccttatgc agttgctctc cagcggttcc atcctctaga ggatagaatg gcgccgggcc | 4680 |
| tttctttatg tttttggcgt cttccatggt ggcgaccggt ggatcccggg ccgcgggtac | 4740 |
| aattccgcag cttttagagc agaagtaaca cttccgtaca ggcctagaag taaaggcaac | 4800 |
| atccactgag gagcagttct ttgatttgca ccaccaccgg atccgggacc tgaaataaaa | 4860 |
| gacaaaaaga ctaaacttac cagttaactt tctggttttt cagttcctcg agtaccggat | 4920 |
| cctctagagt ccggaggctg gatcggtccc ggtgtcttct atggaggtca aaacagcgtg | 4980 |
| gatggcgtct ccaggcgatc tgacggttca ctaaacgagc tctgcttata tagacctccc | 5040 |
| accgtacacg cctaccgccc atttgcgtca atggggcgga gttgttacga cattttggaa | 5100 |
| agtcccgttg attttggtgc caaaacaaac tcccattgac gtcaatgggg tggagacttg | 5160 |
| gaaatccccg tgagtcaaac cgctatccac gcccattgat gtactgccaa aaccgcatca | 5220 |
| ccatggtaat agcgatgact aatacgtaga tgtactgcca agtaggaaag tcccataagg | 5280 |
| tcatgtactg ggcataatgc caggcgggcc atttaccgtc attgacgtca atagggggcg | 5340 |
| tacttggcat atgatacact tgatgtactg ccaagtgggc agtttaccgt aaatactcca | 5400 |
| cccattgacg tcaatggaaa gtccctattg gcgttactat gggaacatac gtcattattg | 5460 |
| acgtcaatgg gcgggggtcg ttgggcggtc agccaggcgg gccatttacc gtaagttatg | 5520 |
| taacggtcga cgatttaaat cagggcgaga cctatcagca cacaatagtc cattatacgc | 5580 |

### 9.1. Polyplex Formulation Procedure 1

Shielded polyplex formulations to study stability, size, toxicity and transfection capacity were prepared in-situ (mixing in a pipette) as follows:

The desired amount of pDNA, cIDNA or mRNA and the calculated amount of the cationic polymer at indicated charge-ratio (+/−) or amine to phosphate ratio (N/P) were diluted in separate tubes in PBS pH 7.4. Only protonatable nitrogens, not amide nitrogens, were considered in the +/− ratio and N/P ratio calculations. Prior to polyplex formation, the corresponding amount of shielding polymer was added and mixed to the nucleic acid test tube. For the shielded polyplex formation, the cationic polymer solution and the genetic material+shielding polymer solution were mixed by rapidly pipetting up and down (ten times) and incubated for 20 min at RT. Then the polyplexes formed were characterized by DLS to determine the size and the Z-potential.

As a particular example, the shielded polyplex PX_{N1__8_V1_1}^{pDNA} loaded with 20 μg of pDNA (final volume of polyplex 200 μl) will be showed. Formulation of other polyplexes are performed in a similar manner. The shielding anionic polymer according to the invention amount is calculated as follows: once stablished the amount of amines required for the NP8 ratio, we have divided them by 2, half of them will be employed for polymer-DNA interaction and the other half will be confronted with the required shielding anionic polymer NP ratio (NP1).

First, the shielding polymer stock solution in water at 10 mg/ml and the polycationic polymer stock solution in water at 4 mg/ml were prepared. The experimental procedure was performed as follows:
1. Add 80 μl of PBS in an Eppendorf tube. Then, dilute 20 μl of pDNA from a stock of 1 mg/ml, and add 15.8 μl of the shielding polymer from a stock of 10 mg/ml (final volume of 115.8 μl).
2. Add 18.2 μl from the polycationic polymer solution (Stock 4 mg/ml) to the pDNA-Shielding polymer solution, and complete with PBS to a final volume of 200 μl (66 μl PBS).
3. Incubate for 20 minutes at room temperature.
4. The polyplex is ready to be used.

Those samples formulated by this first method were prepared in a similar manner for its in-vitro testing. After 24 hours of incubation the toxicity and transfection efficiency will be evaluated. The ratios studied for each polymer were N/P 8, 15 or 30. As a positive control for the transfection jetPEI® (Polyplus-transfection S.A, Illkirch, France) (Ref Polyplus: 101-10N) was used at nitrogen to phosphorus ratio (NP5). Cell transfection was performed using jetPEI® according to the manufacturer's instructions. jetPEI® is mainly composed of a linear polyethylenimine manufactured at Polyplus-transfection. jetPEI® is provided as a 7.5 mM solution in sterile and apyrogenic water (expressed as concentration of nitrogen residues).

### 9.2. Polyplex Formulation Procedure 2

A microfluidic device was used for this procedure of polyplex formulation. The microfluidic device is placed in a laminar flow hood to avoid possible contamination of the samples and all the polymers used in this formulation step were previously sterilized by passing them through 0.22 μm PES filter. For all microfluidics experiments, the microfluidic device was at room temperature.

As a particular example, the shielded polyplex PX_{N1_30_V1_1}^{cIDNA} loaded with 3 μg of cIDNA (final volume of polyplex 200 μl) will be showed. Formulation of other polyplexes are performed in a similar manner. First, the shielding polymer stock solution in water at 50 mg/ml, and the polycationic polymer stock solution in water at 10 mg/ml were prepared. The experimental procedure was performed as follows:
1. Add 75.81 μl of PBS in an Eppendorf tube. Then, dilute 24.19 μl of cIDNA from a stock of 0.124 mg/ml, and add 1.78 μl of the shielding polymer from a stock of 50 mg/ml (final volume of 101.78 μl).
2. In other Eppendorf tube add 94.13 μl of PBS and dilute 4.09 μl from the polycationic polymer solution (Stock 10 mg/ml) (final volume of 98.22 μl).
3. Then, both solutions were loaded into 1 ml plastic syringes (BD Plastipak™, Spain). The two programmable infusion syringe pumps (NE-4000, Syringe Pump, USA) were then used for fluidic injection and control according to the desired flow-rate ratio (300 μl/min) of the side streams to the central stream.
4. the final polyplex solution was collected and allowed to stabilize for 20 min.
5. The polyplex is ready to be used.

### 9.2.A. Microfluidic Device and Setup

A microfluidic device was purchased in Little Things Factory GmbH (Germany). The system consists in two connected reactors made of borosilicate glass: first reactor (LTF-MS) presents 2 inlet-channels (one for the DNA and the other for the polymer) and 1 outlet-channel, Volume 0.2 ml, Channel size: 1 mm, 0.5-20 ml/min/channel, Not sensitive to blockage. Size: 115×60×6 mm (l, w, h). The second reactor (LTF-VS) has 1 inlet-channel (connected to the outlet-channel of the first reactor) and 1 outlet-channel, Volume 1.1 ml, Channel size: 1 mm. Size: 115×60×6 mm (l, w, h). First reactor is employed for the mix and formation of the polyplexes, and the second reactor is used to increase the residence time.

In addition, two programmable pumps control the fluid flow rates of the syringes (NE-4000 Programmable 2 Channel Syringe Pump, Syringe Pump, USA). The system accepts infusion rates from 1.436 μL/hr (1 mL syringe) to 7515 mL/hr (60 mL syringe).

This methodology provides reproducibility to the formation of polyplexes as well as the possibility of scaling up the process.

### Example 10. Size and Stability of Shielded Polyplexes

Stability of polyplexes is a paramount aspect on developing efficient therapies. Ensuring mid-long term stability of the drug product formulation is followed by a panel assay to mimic the physiological conditions that the drug will meet following the administration route where they need be stable during circulation to the target site of action. It is well known that polyplexes displaying positive surface charges undergo salt-induced agglomeration which might cause inaccurate cell biology evaluation and severe toxicity issues when applied systemically. Initial stability studies are currently under development during the present project, they are aimed to monitor polyplex particle characteristics (size).

Size and Z-pot of the stabilized polyplexes formed with cIDNA, mRNA or pDNA at different N/P ratios, different polycations and shielding polymer (V1 or V0.5) were performed using a Malvern ZetasizerNanoZS instrument, equipped with a 532 nm laser at a fixed scattering angle of 173°. 20 μl of the samples were measured using a quartz glass high performance cuvette (Hellma Analytics). Size distribution was measured (diameter, nm) with n >3 measurements. For the stability measurements, the polyplexes were kept in the fridge during the experiment and the stability of the polyplexes was measured at different times.

The sequence of the cIDNA indicated in this example, SEQ ID NO. 1, is that of Table 18 above.

### 10.1. Stabilization of Polyplexes Formed by N1 and V1

The stability at different times and the formation of the N1 polyplexes by polyplex formulation procedure 1 (as reported above, example 8.1), using different NP ratios (8 and 15) and using shielding polymer V1 different +/− charge ratio in PBS pH7.4 were studied. For this experiment, different amounts of genetic material were used too (shown in table 19). The final polyplex solution (200 μl) was allowed to stabilize for 20 min before measuring the size by DLS (Malvern Panalytical, Spain). The polyplexes were kept in the fridge during the experiment, and the stability of the polyplexes was measured at different times.

As shown in table 19, the presence of the shielding polymer provided enhanced stability to the polyplex in solution up to, at least, several days, maintaining a constant size over time and avoiding aggregation.

TABLE 19
Stabilization of polyplexes formed by N1 by different amounts of V1 in
different genetic material and different masses of genetic material.

| | Genetic | | | | |
|---|---|---|---|---|---|
| | material | | D(n)/nm | D(n)/nm | D(n)/nm |
| Polyplex | mass | | t = 0 h | t = 24 h | t = 4 days |
| PX_{N1}_{_{—}}₈_{_{—}}_{V1}_{_{—}}₀^{pDNA} | 150 | ng | 1440 ± 20 | N/A | N/A |
| PX_{N1}_{_{—}}₈_{_{—}}_{V1}_{_{—}}₁^{pDNA} | 150 | ng | 156 ± 18 | 172 ± 13 | 145 ± 23 |
| PX_{N1}_{_{—}}₈_{_{—}}_{V1}_{_{—}}_{0.5}^{pDNA} | 150 | ng | 898 ± 18 | 659 ± 65.2 | 529 ± 36 |
| PX_{N1}_{_{—}}₈_{_{—}}_{V1}_{_{—}}₀^{mRNA} | 150 | ng | 1780 ± 247 | NA | NA |
| PX_{N1}_{_{—}}₈_{_{—}}_{V1}_{_{—}}₁^{mRNA} | 150 | ng | 208 ± 23 | 240 ± 25 | 242 ± 121 |
| PX_{N1}_{_{—}}₈_{_{—}}_{V1}_{_{—}}_{0.5}^{mRNA} | 150 | ng | 1220 ± 98 | 837 ± 55 | 556 ± 313 |
| PX_{N1}_{_{—}}₈_{_{—}}_{V1}_{_{—}}₀^{pDNA} | 1 | μg | N/A | N/A | N/A |
| PX_{N1}_{_{—}}₈_{_{—}}_{V1}_{_{—}}₁^{pDNA} | 1 | μg | 80 ± 7 | 76 ± 7 | 88 ± 3 |
| PX_{N1}_{_{—}}₈_{_{—}}_{V1}_{_{—}}_{0.5}^{pDNA} | 1 | μg | 174 ± 8 | 176 ± 5 | 173 ± 8 |
| PX_{N1}_{_{—}}₈_{_{—}}_{V1}_{_{—}}₀^{mRNA} | 1 | μg | NA | NA | NA |
| PX_{N1}_{_{—}}₈_{_{—}}_{V1}_{_{—}}₁^{mRNA} | 1 | μg | 71 ± 14 | 67 ± 14 | 79 ± 11 |
| PX_{N1}_{_{—}}₈_{_{—}}_{V1}_{_{—}}_{0.5}^{mRNA} | 1 | μg | 137 ± 6 | 136 ± 28 | 140 ± 5 |
| PX_{N1}_{_{—}}₈_{_{—}}_{V1}_{_{—}}₀^{pDNA} | 3 | μg | N/A | N/A | N/A |
| PX_{N1}_{_{—}}₈_{_{—}}_{V1}_{_{—}}₁^{pDNA} | 3 | μg | 92 ± 2 | 93 ± 8 | 92 ± 2 |
| PX_{N1}_{_{—}}₈_{_{—}}_{V1}_{_{—}}_{0.5}^{pDNA} | 3 | μg | 131 ± 19 | 146 ± 7 | 140 ± 30 |

D(n) stands for hydrodynamic diameter measured by DLS; N/A stands for not able to be measured because aggregation was present.

As can be observed in the table, the size of the polyplexes depend on the mass of genetic material and the ratio of shielding polymer present in the final formulation. Those polyplexes formulated without shielding polymer (i.e. PX_{N1_8_V1_0}^{pDNA} and PX_{N1_V1_0}^{MRNA}) gave big aggregates unable to be measured by the DLS technique.

### 10.2. Stabilization of Polyplexes Formed by N1 and V2

As shown in table 20, the presence of the shielding polymer provided enhanced stability to the polyplex formulated by the procedure depicted in example 8.1 in solution up to, at least, several days, maintaining a constant size over time and avoiding aggregation.

TABLE 20
Stabilization of polyplexes formed by N1 by different
amounts of V2 in different genetic material
and different masses of genetic material.

| | Genetic | | | |
|---|---|---|---|---|
| | material | D(n)/nm | D(n)/nm | D(n)/nm |
| Polyplex | mass | t = 0 h | t = 24 h | t = 5 days |
| PX_{N1}_{_{—}}₈_{_{—}}_{V2}_{_{—}}₀^{pDNA} | 1 μg | N/A | N/A | N/A |
| PX_{N1}_{_{—}}₁₅_{_{—}}_{V2}_{_{—}}_{0.5}^{pDNA} | 1 μg | 183 ± 14 | 139 ± 10 | 167 ± 8 |
| PX_{N1}_{_{—}}₈_{_{—}}_{V2}_{_{—}}_{0.5}^{pDNA} | 1 μg | 106 ± 11 | 107 ± 9 | 100 ± 5 |
| PX_{N1}_{_{—}}₈_{_{—}}_{V2}_{_{—}}₁^{pDNA} | 1 μg | 101 ± 4 | 99 ± 1 | 103 ± 7 |
| PX_{N1}_{_{—}}₁₅_{_{—}}_{V2}_{_{—}}₁^{pDNA} | 1 μg | 84 ± 5 | 80 ± 1 | 87 ± 4 |

D(n) stands for hydrodynamic diameter measured by DLS; N/A stands for not able to be measured because aggregation was present.

### 10.3. Stabilization of Polyplexes Formed by N1 and W1

As shown in table 21, the presence of the shielding polymer provided enhanced stability to the polyplex formulated by the procedure depicted in example 6.1 in solution up to, at least, several days, maintaining a constant size over time and avoiding aggregation.

TABLE 21
Stabilization of polyplexes formed by N1 by different
amounts of W1 in different genetic material
and different masses of genetic material.

| | Genetic | | | |
|---|---|---|---|---|
| | material | D(n)/nm | D(n)/nm | D(n)/nm |
| Polyplex | mass | t = 0 h | t = 24 h | t = 4 days |
| PX_{N1}_{_{—}}₈_{_{—}}_{W1}_{_{—}}₀^{pDNA} | 1 μg | N/A | N/A | N/A |
| PX_{N1}_{_{—}}₁₅_{_{—}}_{W1}_{_{—}}_{0.5}^{pDNA} | 1 μg | 54 ± 6 | 60 ± 2 | 60 ± 2 |
| PX_{N1}_{_{—}}₈_{_{—}}_{W1}_{_{—}}_{0.5}^{pDNA} | 1 μg | 63 ± 3 | 73 ± 4 | 76 ± 4 |
| PX_{N1}_{_{—}}₈_{_{—}}_{W1}_{_{—}}₁^{pDNA} | 1 μg | 54 ± 5 | 57 ± 10 | 68 ± 5 |
| PX_{N1}_{_{—}}₁₅_{_{—}}_{W1}_{_{—}}₁^{pDNA} | 1 μg | 45 ± 5 | 47 ± 1 | 47 ± 2 |

D(n) stands for hydrodynamic diameter measured by DLS; N/A stands for not able to be measured because aggregation was present.

### 10.4 Stabilization of Polyplexes Formed by N1 and Y1

As shown in table 22, the presence of the shielding polymer provided stability to the polyplex formulated by the procedure depicted in example 6.1 in solution

TABLE 22
Polyplex formed by N1 and Y1 using pDNA as genetic material.

| | Genetic | | |
|---|---|---|---|
| | material | D(n)/nm | D(n)/nm |
| Polyplex | mass | t = 0 h | t = 72 h |
| PX_{N1}_{_{—}}₁₅_{_{—}}_{Y1}_{_{—}}₀^{pDNA} | 150 ng | N/A | N/A |
| PX_{N1}_{_{—}}₁₅_{_{—}}_{Y1}_{_{—}}₂^{pDNA} | 150 ng | 99.2 ± 0.566 | 75.6 ± 21 |

### 10.5 Stabilization of Polyplexes Formed by N1 and F1

As shown in table 23, the presence of the shielding polymer provided stability to the polyplex formulated by the procedure depicted in example 9.1.

TABLE 23
Polyplex formed by N1 and F1 using pDNA and mRNA
as genetic material and different masses.

| | Genetic | | | |
|---|---|---|---|---|
| | material | | D(n)/nm | D(n)/nm |
| Polyplex | mass | | t = 0 h | t = 24 h |
| PX_{N1}_{_{—}}₁₅_{_{—}}_{F1}_{_{—}}₂^{pDNA} | 150 | ng | 224 ± 14 | 342 ± 7 |
| PX_{N1}_{_{—}}₁₅_{_{—}}_{F1}_{_{—}}₂^{pDNA} | 1 | ug | 178 ± 34 | 201 ± 47 |
| PX_{N1}_{_{—}}₁₅_{_{—}}_{F1}_{_{—}}₂^{mRNA} | 150 | ng | 170 ± 8 | 180 ± 78 |
| PX_{N1}_{_{—}}₁₅_{_{—}}_{F1}_{_{—}}₂^{mRNA} | 1 | ug | 128 ± 71 | 199 ± 23 |

### 10.6 Stabilization of Polyplexes Formed by N1 and Comparative Example E1

As shown in table 24, the presence of the shielding polymer didn't provide stability to the polyplex formulated by the procedure depicted in example 6.1 in solution

TABLE 24
Polyplexes formed by N1 by different amounts of E1 in different
genetic material and different masses of genetic material.

| | Genetic | |
|---|---|---|
| | material | D(n)/nm |
| Polyplex | mass | t = 0 h |
| PX_{N1}_{_{—}}₈_{_{—}}_{E1}_{_{—}}₀^{pDNA} | 150 ng | N/A |
| PX_{N1}_{_{—}}₈_{_{—}}_{E1}_{_{—}}₁^{pDNA} | 150 ng | N/A |
| PX_{N1}_{_{—}}₈_{_{—}}_{E1}_{_{—}}₀^{mRNA} | 150 ng | N/A |
| PX_{N1}_{_{—}}₈_{_{—}}_{E1}_{_{—}}₁^{mRNA} | 150 ng | N/A |
| PX_{N1}_{_{—}}₈_{_{—}}_{E1}_{_{—}}₀^{pDNA} | 1 μg | N/A |
| PX_{N1}_{_{—}}₈_{_{—}}_{E1}_{_{—}}₁^{pDNA} | 1 μg | N/A |
| PX_{N1}_{_{—}}₈_{_{—}}_{E1}_{_{—}}₀^{mRNA} | 1 μg | NA |
| PX_{N1}_{_{—}}₈_{_{—}}_{E1}_{_{—}}₁^{mRNA} | 1 μg | N/A |
| PX_{N1}_{_{—}}₁₅_{_{—}}_{E1}_{_{—}}₀^{pDNA} | 150 ng | N/A |
| PX_{N1}_{_{—}}₁₅_{_{—}}_{E1}_{_{—}}₁^{pDNA} | 150 ng | N/A |
| PX_{N1}_{_{—}}₁₅_{_{—}}_{E1}_{_{—}}₀^{mRNA} | 150 ng | N/A |
| PX_{N1}_{_{—}}₁₅_{_{—}}_{E1}_{_{—}}₁^{mRNA} | 150 ng | N/A |
| PX_{N1}_{_{—}}₁₅_{_{—}}_{E1}_{_{—}}₀^{pDNA} | 1 μg | N/A |
| PX_{N1}_{_{—}}₁₅_{_{—}}_{E1}_{_{—}}₁^{pDNA} | 1 μg | N/A |
| PX_{N1}_{_{—}}₁₅_{_{—}}_{E1}_{_{—}}₀^{mRNA} | 1 μg | NA |
| PX_{N1}_{_{—}}₁₅_{_{—}}_{E1}_{_{—}}₁^{mRNA} | 1 μg | N/A |

N/A stands for not able to be measured because aggregation was present

### 10.7 Polyplex Z Potential Variation Upon V1 Shielding Polymer Addition to Polyplexes Formed by N1

Z-potential is a key feature of the polyplexes in order to ensure the shielding and the aggregation avoidance. Increasing amounts of shielding V1 was used to shield the same polyplex formed by N1 and pDNA following formulation protocol 1. Despite the size stabilization and the aggregation prevention, the decrease in Z-potential as shielding polymer ratio increases can be clearly seen. This diminution in Z-pot confirms that the positive charge shielding was efficiently achieved (Table 25).

TABLE 25
Size and Z-potential variation with increasing
amounts of shielding polymer V1.

| | Genetic | | |
|---|---|---|---|
| | material | D(n)/nm | |
| Polyplex | mass | t = 0 h | Z-Pot/mV |
| PX_{N1}_{_{—}}₈_{_{—}}_{V1}_{_{—}}₀^{pDNA} | 1 μg | NA | 12.3 ± 1.0 |
| PX_{N1}_{_{—}}₈_{_{—}}_{V1}_{_{—}}₁^{pDNA} | 1 μg | 80 ± 7 | 0.10 ± 0.3 |
| PX_{N1}_{_{—}}₈_{_{—}}_{V1}_{_{—}}_{0.5}^{pDNA} | 1 μg | 174 ± 8 | −0.07 ± 0.03 |

D(n) stands for hydrodynamic diameter measured by DLS; N/A stands for not able to be measured because aggregation was present.

10.8. Stabilization of Polyplexes Formed by jetPEI Comparing Two Formulation Procedures

The variation of size and stability of stabilized polyplexes formed by jetPEI (NP=5) with pDNA and stabilized with different +/− ratio of shielding polymer V1 and formulation procedure is assessed by DLS. As shown in table 26, the presence of the shielding polymer provided enhanced stability to the polyplex in solution up to 96 h maintaining a constant size over time and avoiding aggregation.

TABLE 26
Size variation with increasing amounts of shielding polymers V1

| | Genetic | | | |
|---|---|---|---|---|
| | material | D(n)/nm | D(n)/nm | D(n)/nm |
| Polyplex | mass | t = 0 h | t = 24 h | t = 96 h |
| PX_{jetPEI}_{_{—}}₅_{_{—}}_{V1}_{_{—}}₀^{pDNA} | ^{a}150 ng | NA | NA | NA |
| PX_{jetPEI}_{_{—}}₅_{_{—}}_{V1}_{_{—}}_{0.5}^{pDNA} | ^{a}150 ng | 113 ± 3 | 90 ± 25 | 107 ± 12 |
| PX_{jetPEI}_{_{—}}₅_{_{—}}_{V1}_{_{—}}₁^{pDNA} | ^{a}150 ng | 109 ± 12 | 120 ± 20 | 133 ± 7 |
| PX_{jetPEI}_{_{—}}₅_{_{—}}_{V1}_{_{—}}₀^{pDNA} | ^{b}150 ng | NA | NA | NA |
| PX_{jetPEI}_{_{—}}₅_{_{—}}_{V1}_{_{—}}_{0.5}^{pDNA} | ^{b}150 ng | 67 ± 2 | * | 71 ± 5 |
| PX_{jetPEI}_{_{—}}₅_{_{—}}_{V1}_{_{—}}₁^{pDNA} | ^{b}150 ng | 100 ± 11 | * | 99 ± 8 |

D(n) stands for hydrodynamic diameter measured by DLS;
N/A stands for not able to be measured because aggregation was present;
*Not measured but no aggregates present.
^{a}formulation procedure 2; ^{b}formulation procedure 1.

10.9. Stabilization of Polyplexes Formed by jetPEI with E1

The variation of size and stability of stabilized polyplexes formed by jetPEI (NP=5) with pDNA and stabilized with different +/− ratio of shielding polymer E1 and formulation procedure 1 (described in section 8.1) was assessed by DLS. As shown in table 27, the presence of the shielding polymer didn't provided stability to the polyplex in solution.

TABLE 27
Size variation with increasing amounts of shielding polymers E1

| | Genetic | | |
|---|---|---|---|
| | material | D(n)/nm | D(n)/nm |
| Polyplex | mass | t = 0 h | t = 24 h |
| PX_{jetPEI}_{_{—}}₅_{_{—}}_{E1}_{_{—}}₀^{pDNA} | 150 ng | N/A | N/A |
| PX_{jetPEI}_{_{—}}₅_{_{—}}_{E1}_{_{—}}_{0.5}^{pDNA} | 150 ng | N/A | N/A |
| PX_{jetPEI}_{_{—}}₅_{_{—}}_{E1}_{_{—}}₁^{pDNA} | 150 ng | N/A | N/A |

D(n) stands for hydrodynamic diameter measured by DLS;
N/A stands for not able to be measured because aggregation was present

### 10.10. Stabilization of Polyplexes Formed by N2 and V1

The stability at different times and the formation of the N2 polyplexes by polyplex formulation procedure 1 (as reported above), using NP ratio of 8 and using shielding polymer V1 different +/− charge ratio in PBS pH7.4 were studied. The presence of the shielding polymer provided enhanced stability to the polyplex in solution both with pDNA and mRNA, maintaining a constant size over time and avoiding aggregation (Table 28).

TABLE 28
N2 Polyplex Size variation with increasing
amounts of shielding polymers V1 with time.

| | Genetic | | | |
|---|---|---|---|---|
| | material | D(n)/nm | D(n)/nm | D(n)/nm |
| Polyplex | mass | t = 0 h | t = 24 h | t = 96 h |
| PX_{N2}_{_{—}}₈_{_{—}}_{V1}_{_{—}}₀^{pDNA} | 3 μg | NA | NA | NA |
| PX_{N2}_{_{—}}₈_{_{—}}_{V1}_{_{——}}₁^{pDNA} | 3 μg | 85 ± 2 | 81.2 ± 3.59 | 91 ± 7 |
| PX_{N2}_{_{—}}₈_{_{—}}_{V1}_{_{—}}_{0.5}^{pDNA} | 3 μg | 120 ± 6 | 128 ± 16.4 | 132 ± 11 |
| PX_{N2}_{_{—}}₈_{_{—}}_{V1}_{_{—}}₀^{mRNA} | 1 μg | NA | NA | NA |
| PX_{N2}_{_{—}}₈_{_{—}}_{V1}_{_{—}}₁^{mRNA} | 1 μg | 60 ± 16 | 73.5 ± 12.6 | 72 ± 3 |
| PX_{N2}_{_{—}}₈_{_{—}}_{V1}_{_{—}}_{0.5}^{mRNA} | 1 μg | 131 ± 24 | 163 ± 11.4 | 159 ± 8 |
| PX_{N2}_{_{—}}₁₅_{_{—}}_{V1}_{_{—}}₀^{mRNA} | 1 μg | NA | NA | NA |
| PX_{N2}_{_{—}}₁₅_{_{—}}_{V1}_{_{—}}₁^{mRNA} | 1 μg | 36 ± 3 | 39 ± 3 | 41 ± 4 |
| PX_{N2}_{_{—}}₁₅_{_{—}}_{V1}_{_{—}}_{0.5}^{mRNA} | 1 μg | 64 ± 3 | 68 ± 7 | 65 ± 2 |

D(n) stands for hydrodynamic diameter measured by DLS; N/A stands for not able to be measured because aggregation was present.

10.11. Stabilization of Polyplexes Formed by N1 and cIDNA

The polyplexes formed by N1 and cIDNA stabilisation was performed using a microfluidic device. The stability and formation of the polyplexes was using NP ratio of 30 for polyplex formation and NP ratio of 1 for shielding polymer (V1), in PBS pH 7.4. As shown in table 29, the presence of the shielding polymer provided enhanced stability to the polyplex in solution.

The sequence of the cIDNA indicated in this example, SEQ ID NO. 1, is that of Table 18 above.

TABLE 29
N1 Polyplex Size with time with a +/− ratio = 1 of V1.

| | Genetic | | |
|---|---|---|---|
| | material | D(n)/nm | D(n)/nm |
| Polyplex | mass | t = 0 h | t = 24 h |
| PX_{N1}_{_{—}}₃₀_{_{—}}_{V1}_{_{—}}₁^{cIDNA} | 3 μg | 80 ± 6 | 88 ± 4 |

D(n) stands for hydrodynamic diameter measured by DLS.

### Example 11. Complexation/Disassembly Experiments

In addition, the complexation efficacy and possible presence of free pDNA in the polyplexes were assessed using an electrophoresis gel as first screening method. To perform the electrophoresis, E-gel Power Snap Electrophoresis Device and E-Gel Power Snap Camera (Invitrogen) was used. 1.2% agarose gels prepared that include the SYBR safe DNA marker (E-Gel® 1.2% with SYBR safe, Invitrogen) were used. The complexation efficiency of the polyplexes (20 μl) at different NPs and different +/− shielding polymer ratio were evaluated, and also the disassembly of the polyplexes in the presence of low heparin (0.075|U/ml) and high heparin (200 IU/ml) concentration (PanReacAppliChem, Spain). For the low concentration, 0.1 μl of a 151 U/ml heparin solution was added to the 20 μl of already formed polyplex, and for the high concentration, 0.8 μl of a 50001 U/ml heparin solution to the 20 μl of polyplex was added. Once the gel is loaded (20 μl/well), select the equipment protocol according to the type of gel used (in our case, protocol approximately 40 min, although the time can be modified according to the samples).

TABLE 30
Complexation and disassembly experiments representative
of formulated polyplexes synthesized.

| | Genetic | | |
|---|---|---|---|
| | material | | |
| Compound | mass | Complexation | Disassembly test |
| PX_{N1}_{_{—}}₈_{_{—}}_{V2}_{_{—}}₀^{pDNA} | 1 μg | YES | YES (high heparin) |
| PX_{N1}_{_{—}}₁₅_{_{—}}_{V2}_{_{—}}_{0.5}^{pDNA} | 1 μg | YES | YES (high heparin) |
| PX_{N1}_{_{—}}₈_{_{—}}_{V2}_{_{—}}_{0.5}^{pDNA} | 1 μg | YES | YES (high heparin) |
| PX_{N1}_{_{—}}₈_{_{—}}_{V2}_{_{—}}₁^{pDNA} | 1 μg | YES | YES (high heparin) |
| PX_{N1}_{_{—}}₈_{_{—}}_{V1}_{_{—}}₀^{pDNA} | 1 μg | YES | YES (high heparin) |
| PX_{N1}_{_{—}}₈_{_{—}}_{V1}_{_{—}}₁^{pDNA} | 1 μg | YES | YES (high heparin) |
| PX_{N1}_{_{—}}₈_{_{—}}_{V1}_{_{—}}_{0.5}^{pDNA} | 1 μg | YES | YES (high heparin) |
| PX_{N1}_{_{—}}₈_{_{—}}_{V1}_{_{—}}₀^{mRNA} | 1 μg | YES | YES (high heparin) |
| PX_{N1}_{_{—}}₈_{_{—}}_{V1}_{_{—}}₁^{mRNA} | 1 μg | YES | YES (high heparin) |
| PX_{N1}_{_{—}}₈_{_{—}}_{V1}_{_{—}}_{0.5}^{mRNA} | 1 μg | YES | YES (high heparin) |
| PX_{N1}_{_{—}}₈_{_{—}}_{W1}_{_{—}}_{0.5}^{pDNA} | 1 μg | YES | YES (high heparin) |
| PX_{N1}_{_{—}}₈_{_{—}}_{W1}_{_{—}}₁^{pDNA} | 1 μg | YES | YES (high heparin) |
| PX_{N1}_{_{—}}₁₅_{_{—}}_{W1}_{_{—}}_{0.5}^{pDNA} | 1 μg | YES | YES (high heparin) |
| PX_{N1}_{_{—}}₁₅_{_{—}}_{W1}_{_{—}}₁^{pDNA} | 1 μg | YES | YES (high heparin) |

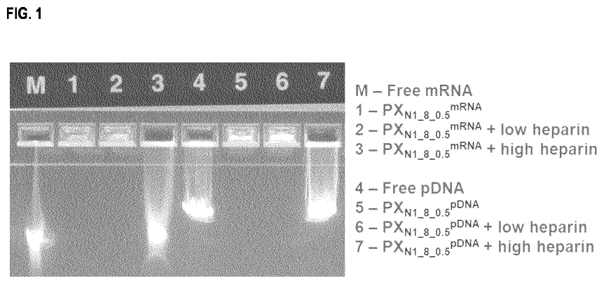

In all cases, no free pDNA is observed at the different NPs or at low concentrations of heparin. However, at high concentration of heparin, free pDNA signal was observed due to the competition between heparin and pDNA to bind to the polymer, showing the ability of the polymer to release their cargo (Representative image of the gels can be observed in FIG. 1).

### Example 12. In Vitro Biological Studies

### Example 12A. Cell Culture

HeLa cells were cultured in DMEM high glucose with Glutamax (Gibco-Thermo Fisher #61965-059) supplemented with 10% of Fetal Bovine Serum (Hyclone #SV30160.03HI, provided by GE Healthcare Europe GmbH). Transfections were carried out on 96-well plates containing 10000 cells/well in a final volume of 100 μl, and cells were incubated 24 hours at 37° C. and 5% CO2. After 24 h, the medium was removed and refreshed with 90 μl of complete medium. The transfection mixtures were prepared using PBS and in the case of the positive control (JetPEI) manufacturer guidelines were followed (#101-10N, Polyplus Transfection), after 20 min of stabilization 10 μl of each formulation were added to the cells. After 24 hours cells were recovered and processed.

HEK293 (Human embryonic kidney) cells were cultured in DMEM high glucose (Gibco ref 61965-059)+10% FBS ((Hyclone #SV30160.03HI, provided by GE Healthcare Europe GmbH). Transfections were carried out on 96-well plates containing 10000 cells/well in a final volume of 100 μl, and cells were incubated 24 hours at 37° C. and 5% CO2. After 24 h, the medium was removed and refreshed with 90 μl of complete medium. The transfection mixtures were prepared using PBS and in the case of the positive control (JetPEI) manufacturer guidelines were followed (#101-10N, Polyplus Transfection), after 20 min of stabilization 10 μl of each formulation were added to the cells. After 24 hours cells were recovered and processed Example 12B. ATP Evaluation for Cell Toxicity evaluation

After 24 h post-incubation, the medium was aspirated and 50 μl/well of ATPLite reagent (ATPLite PerkinElmer #6016731) were added. The plate was incubated 10 minutes at room temperature in the dark. Luminiscence was read spectrophotometrically using VictorNivo (PerkinElmer) and data was represented as the percentage of cell viability, taken untreated control cells as 100%.

### Example 12C. Luciferase Assay

After 24 h post-incubation, 100 μl of BrightGlo reagent (Promega #E2620) was added in each well following manufacturer instructions. After 5 minutes of incubation at room temperature luciferase activity was measured using VictorNivo (PerkinElmer). Data was represented as luminescence relative to the percentage of transfection relative to the positive control of transfection.

### Example 12D. Biological Activity of Polyplexes Formed by N1 and V1 in HeLa Cells

The transfection efficiency and the cell viability of the polyplexes formed by N1 and V1 in HeLa cells is reported in the following table. The transfection data is represented as % of the positive control. jetPEI® being the positive control 100% after 24 h of treatment and cell viability is compared to non-treated (NT) cells, being the ATP content readout of NT cells equal to 100%.

TABLE 31
Cell viability and transfection

| | Genetic | | | |
|---|---|---|---|---|
| | material | | | |
| Polyplex | mass | | Cell viability | Transfection |
| PX_{N1}_{_{—}}₈_{_{—}}_{V1}_{_{—}}₀^{mRNA} | 150 | ng | 116.2 ± 3 | 0.03 ± 0.01 |
| PX_{N1}_{_{—}}₈_{_{—}}_{V1}_{_{—}}₁^{mRNA} | 150 | ng | 121 ± 3 | 0.05 ± 0.01 |
| PX_{N1}_{_{—}}₈_{_{—}}_{V1}_{_{—}}_{0.5}^{mRNA} | 150 | ng | 117 ± 5 | 0.10 ± 0.01 |
| PX_{N1}_{_{—}}₈_{_{—}}_{V1}_{_{—}}₀^{mRNA} | 1 | μg | 104 ± 21 | 4 ± 3 |
| PX_{N1}_{_{—}}₈_{_{—}}_{V1}_{_{—}}₁^{mRNA} | 1 | μg | 103 ± 20 | 15 ± 19 |
| PX_{N1}_{_{—}}₈_{_{—}}_{V1}_{_{—}}_{0.5}^{mRNA} | 1 | μg | 107 ± 6 | 18 ± 19 |
| PX_{N1}_{_{—}}₈_{_{—}}_{V1}_{_{—}}₀^{pDNA} | 150 | ng | 107.8 ± 1.2 | 0.7 ± 0.4 |
| PX_{N1}_{_{—}}₈_{_{—}}_{V1}_{_{—}}₁^{pDNA} | 150 | ng | 114 ± 6 | 0.6 ± 0.7 |
| PX_{N1}_{_{—}}₈_{_{—}}_{V1}_{_{—}}_{0.5}^{pDNA} | 150 | ng | 114 ± 3 | 0.5 ± 0.2 |
| PX_{N1}_{_{—}}₈_{_{—}}_{V1}_{_{—}}₀^{pDNA} | 1 | μg | 91 ± 8 | 282 ± 19 |
| PX_{N1}_{_{—}}₈_{_{—}}_{V1}_{_{—}}₁^{pDNA} | 1 | μg | 103 ± 17 | 273 ± 164 |
| PX_{N1}_{_{—}}₈_{_{—}}_{V1}_{_{—}}_{0.5}^{pDNA} | 1 | μg | 96 ± 10 | 480 ± 40 |

### Example 12E. Biological Activity of Polyplexes Formed by N1 and V2 in HeLa Cells

The transfection efficiency and the cell viability of the polyplexes formed by N1 and V2 in HeLa cells is reported in the following table. The transfection data is represented as % of the positive control. jetPEI® being the positive control 100% after 24 h of treatment and cell viability is compared to non-treated (NT) cells, being the ATP content readout of NT cells equal to 100%.

TABLE 32
Cell viability and transfection

| | Genetic | | |
|---|---|---|---|
| | material | | |
| Polyplex | mass | Cell viability | Transfection |
| PX_{N1}_{_{—}}₈_{_{—}}_{V2}_{_{—}}₀^{pDNA} | 1 μg | 94 ± 3 | 118 ± 6 |
| PX_{N1}_{_{—}}₁₅_{_{—}}_{V2}_{_{—}}_{0.5}^{pDNA} | 1 μg | 94 ± 13 | 182 ± 119 |
| PX_{N1}_{_{—}}₈_{_{—}}_{V2}_{_{—}}_{0.5}^{pDNA} | 1 μg | 76 ± 2 | 260 ± 46 |
| PX_{N1}_{_{—}}₈_{_{—}}_{V2}_{_{—}}₁^{pDNA} | 1 μg | 73 ± 6 | 376 ± 14 |
| PX_{N1}_{_{—}}₁₅_{_{—}}_{V2}_{_{—}}₁^{pDNA} | 1 μg | 87 ± 20 | 498 ± 60 |

### Example 12F. Biological Activity of Polyplexes Formed by N1 and W1 in HeLa Cells

The transfection efficiency and the cell viability of the polyplexes formed by N1 and W1 in HeLa cells is reported in the following table. The transfection data is represented as % of the positive control. jetPEI® being the positive control 100% after 24 h of treatment and cell viability is compared to non-treated (NT) cells, being the ATP content readout of NT cells equal to 100%.

TABLE 33
Cell viability and transfection

| | Genetic | | |
|---|---|---|---|
| | material | | |
| Polyplex | mass | Cell viability | Transfection |
| PX_{N1}_{_{—}}₈_{_{—}}_{W1}_{_{—}}₀^{pDNA} | 1 μg | 91 ± 7 | 282 ± 19 |
| PX_{N1}_{_{—}}₁₅_{_{—}}_{W1}_{_{—}}₀^{pDNA} | 1 μg | 90 ± 9 | 166 ± 3 |
| PX_{N1}_{_{—}}₁₅_{_{—}}_{W1}_{_{—}}_{0.5}^{pDNA} | 1 μg | 85 ± 12 | 892 ± 15 |
| PX_{N1}_{_{—}}₈_{_{—}}_{W1}_{_{—}}_{0.5}^{pDNA} | 1 μg | 101 ± 4 | 1121 ± 42 |
| PX_{N1}_{_{—}}₈_{_{—}}_{W1}_{_{—}}₁^{pDNA} | 1 μg | 94.0 ± 1.1 | 919 ± 67 |
| PX_{N1}_{_{—}}₁₅_{_{—}}_{W1}_{_{—}}₁^{pDNA} | 1 μg | 86.9 ± 0.7 | 877 ± 22 |

As can be extracted from the data described above, the presence of the shielding polymer not only increases the cell viability in HeLa cells, conferring to the polymer complex less toxicity but increases in a outstanding manner the transfection efficiency up to 10 fold.

### Example 12G. Biological Activity of Polyplexes Formed by N1 and V1 in HEK293 Cells

The transfection efficiency and the cell viability of the polyplexes formed by N1 and V1 in HEK293 cells is reported in the following table. The transfection data is represented as % of the positive control. jetPEI® being the positive control 100% after 24 h of treatment and cell viability is compared to non-treated (NT) cells, being the ATP content readout of NT cells equal to 100%.

TABLE 34
Cell viability and transfection

| | Genetic | | |
|---|---|---|---|
| | material | | |
| Polyplex | mass | Cell viability | Transfection |
| PX_{N1}_{_{—}}₁₅_{_{—}}_{V1}_{_{—}}₀^{pDNA} | 1 μg | 105 ± 3 | 305 ± 40 |
| PX_{N1}_{_{—}}₁₅_{_{—}}_{V1}_{_{—}}_{0.5}^{pDNA} | 1 μg | 83 ± 1 | 467 ± 7 |
| PX_{N1}_{_{—}}₁₅_{_{—}}_{V1}_{_{—}}₁^{pDNA} | 1 μg | 110 ± 16 | 605 ± 18 |

### Example 12H. Biological Activity of Polyplexes Formed by N1 and W1 in HEK293 Cells

The transfection efficiency and the cell viability of the polyplexes formed by N1 and W1 in HEK293 cells is reported in the following table. The transfection data is represented as % of the positive control. jetPEI® being the positive control 100% after 24 h of treatment and cell viability is compared to non-treated (NT) cells, being the ATP content readout of NT cells equal to 100%.

TABLE 35
Cell viability and transfection

| | Genetic | | |
|---|---|---|---|
| | material | | |
| Polyplex | mass | Cell viability | Transfection |
| PX_{N1}_{_{—}}₁₅_{_{—}}_{W1}_{_{—}}₀^{pDNA} | 1 μg | 105 ± 3 | 305 ± 40 |
| PX_{N1}_{_{—}}₁₅_{_{—}}_{W1}_{_{—}}_{0.5}^{pDNA} | 1 μg | 97 ± 10 | 469 ± 2 |
| PX_{N1}_{_{—}}₁₅_{_{—}}_{W1}_{_{—}}₁^{pDNA} | 1 μg | 88 ± 5 | 412 ± 46 |

As can be extracted from the data described above, the presence of the shielding polymer not only increases the cell viability in HEK293 cells, conferring to the polymer complex less toxicity but increases in a outstanding manner the transfection efficiency up to 6 fold.

### REFERENCES CITED IN THE APPLICATION

J Mol Med, 2002, vol. 80, pp. 648-654
T. W. Green and P. G. M. Wuts, Protective Groups in Organic Chemistry, Wiley, 3rd ed. 1999, Chapter 5 (pp. 369-451)
T. W. Green and P. G. M. Wuts, Protective Groups in Organic Chemistry, Wiley, 3rd ed. 1999, Chapter 7 (pp. 495-653)
Chinese Journal of Polymer Science (English Edition), 33(8), 1186-1195

### CLAUSES

For reasons of completeness, various aspects of the invention are set out in the following numbered clauses:
Clause 1. An anionic polymer of formula (Ia) or (Ib), a pharmaceutically acceptable salt thereof, or any stereoisomer or mixtures of stereoisomers, either of the compound of formula (Ia) or (Ib), or of any of its pharmaceutically acceptable salts, comprising homo-polypeptides or random or block or graft co-polypeptides

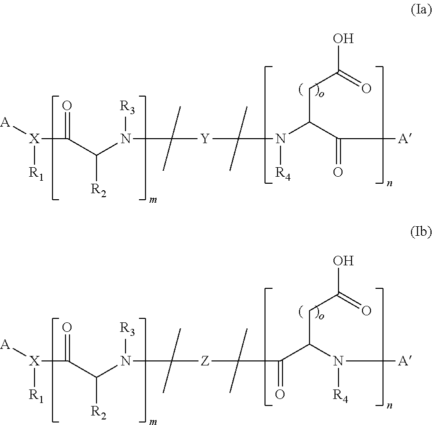

wherein Y is selected from the group consisting of —CO—(CH₂)p-CO— and —CO—(CH₂)q-S—S—(CH₂)r-CO—;
Z is selected from the group consisting of a single bond, —CO—(CH₂)q-S—S—(CH₂)r-NH—(R₅)z-, —CO—(CH₂)p-NH—(R₅)z-, and —(R₅)z-;
o is an integer selected from 1 to 2;
wherein p, q and r are each independently an integer selected from 1-6;
R5 is a random or block co-polymer comprising at least two different repeating units selected from the group consisting of (II), (Ill), (IV), (V), (VI), (VII), (VIII) and (IX):

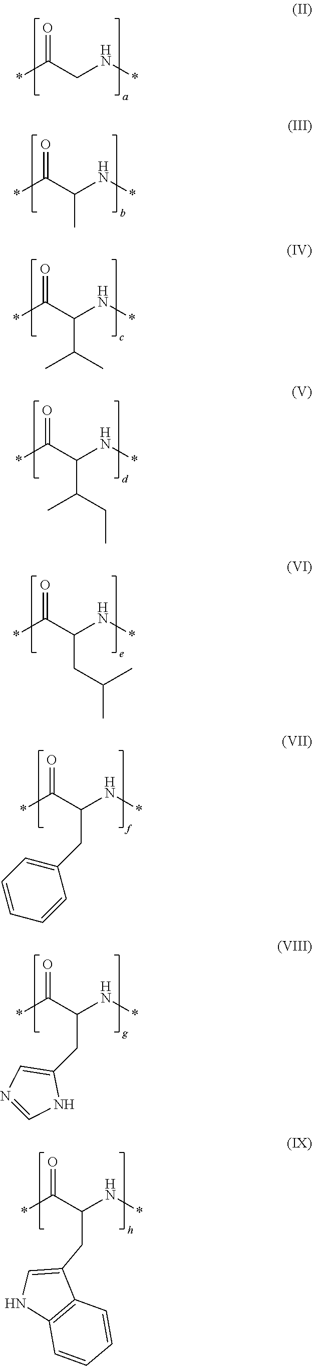

a, b, c, d, e, f, g and h are each independently an integer selected from 0 to 20; with the proviso that at least one of a, b, c, d, e, f, g and h is different to 0;
z is an integer selected from 5 to 100;
X is selected from N, S, and O;
R₁ is selected from the group consisting of H and (C₁-C₆)alkyl, with the proviso that R₁ is absent when X is O;
R₃ and R₄ are each independently selected from the group consisting of H and —CH₃;
m is an integer selected from 5 to 250;
n is an integer selected from 3-200;
with the proviso that the ratio between m:n ranges from 1:8 to 30:1;
R₂ is selected from H, and a radical selected from the group consisting of (X), (XI), (XII), and (XIII)

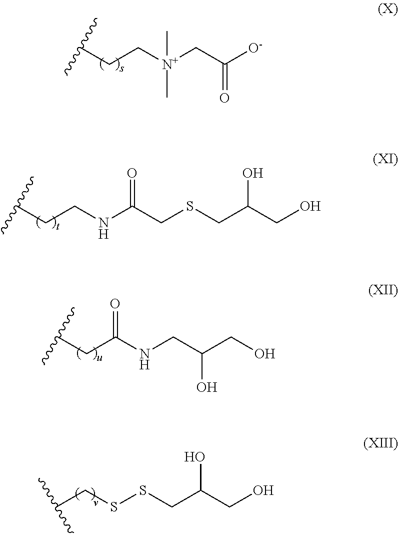

wherein s, t, u, and v are each independently an integer selected from 1 to 4;
wherein “/” indicates that the sequential order of the monomer repeating units defined by square brackets on both sides of the sign is arbitrary, thus though the repeating units defined by square brackets in the formulas Ia and Ib are shown in a particular order for convenience of description, the repeating units may be present in any order and the repeating units may be block or randomly present;
wherein in formula Ib, the sequential order of the respective repeating units of formula (II), (Ill), (IV), (V), (VI), (VII), (VIII) and (IX) of R5 and the repeating units denoted with the square brackets with the n integer, may be block or randomly present;
A and A′ are each independently selected from H, OH, straight chain or branched —(C₁-C₆)alkyl, straight chain or branched —CO(C₁-C₆)alkyl, —(C₅-C₁₀)aryl, —(C₅-C₁₀)heteroaryl, —(C₆-C₁₀)aralkyl, —(C₁-C₆)alkyl-O—(C₅-C₁₀)aryl, —(C₅-C₁₀)heterocycloalkyl, —(C₁-C₁₀)alkoxy, —(C₆-C₁₀)aryloxy, —(C₆-C₁₀)aralkoxy, —(C₅-C₁₀)heteroaralkoxy, —(C₁-C₁₀)alkyl-O—(C₆-C₁₀)aryloxy, an amine protecting group, a natural or unnatural alpha amino acid, a carboxyl protecting group, a labelling or imaging agent and a cell-targeting agent;
being each of A and A′ independently optionally substituted by one or more groups selected from the group consisting of —OH, halogen, —CF₃, —NH₂, —NH—(C₁-C₄)alkyl, —NR₆R₇, —NH—CO—(C₁-C₆)alkyl, —(C₁-C₆)alkyl, —NO₂, —N₃, —CO—(C₁-C₆)alkyl, —CO—O—(C₁-C₆)alkyl, —SO₃H, —SO₂NH₂, —SO₂—N((C₁-C₆)alkyl)₂, —COOH, CONH₂, and —CON((C₁-C₆)alkyl)₂;
R₆ and R₇ are independently selected from the group consisting of H, —(C₁-C₄)alkyl and an amine protecting group.
Clause 2. The anionic polymer according to clause 1, wherein m is an integer selected from 20 to 160, and n is an integer selected from 4 to 100; with the proviso that the ratio between m:n ranges from 1:5 to 10:1.
Clause 3. The anionic polymer according to any of clauses 1-2, which is of formula (Ia) or (Ib), R₃ is —CH₃; and R₂ is H.
Clause 4. The anionic polymer according to any of clauses 1-3, which is of formula (Ib1)

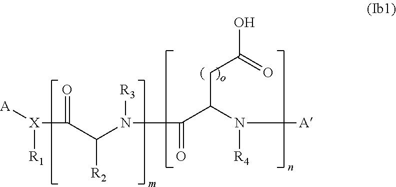

and and R₂ is selected from the group consisting of a radical selected from the group consisting of (X), (XI), (XII), and (XIII).
Clause 5. The anionic polymer according to any of clauses 1-3, which is of formula (Ib1) as defined above, wherein R₂ is H; and R₃ is —CH₃.
Clause 6. The anionic polymer according to any of clauses 1-5, wherein the anionic polymer is of formula (Ib), wherein Z is selected from the group consisting of —CO—(CH₂)q-S—S—(CH₂)r-NH—(R₅)z-, —CO—(CH₂)p-NH—(R₅)z-, and —(R₅)z-; R₅ is a random or block co-polymer comprising at least 2 different repeating units selected from the group consisting of (II), (Ill), (IV), (V), (VI), (VII), (VIII) and (IX); and wherein the sequential order of the respective repeating units of formula (II), (Ill), (IV), (V), (VI), (VII), (VIII) and (IX) of R₅ and the repeating units denoted with the square brackets with the n integer, may be block or randomly present.
Clause 7. The anionic polymer according to any of clauses 1-6, wherein the anionic polymer is of formula Ib; Z is selected from the group consisting of —CO—(CH₂)q-S—S—(CH₂)r-NH—(R₅)z-, —CO—(CH₂)p-NH—(R₅)z-, and —(R₅)z-; and R₅ is a random or block co-polymer comprising at least 2 repeating units selected from the group consisting of (Ill), (VI), (VIII) and (IX); and wherein c, e, g, and h are independently an integer selected from 1 to 20; and z is an integer selected from 5-35.
Clause 8. The anionic polymer according to any of clauses 1-7, wherein the anionic polymer is of formula (Ib), wherein Z is selected from the group consisting of —CO—(CH₂)q-S—S—(CH₂)r-NH—(R₅)z-, —CO—(CH₂)p-NH—(R₅)z-, and —(R₅)z-; R₅ is a random or block co-polymer comprising at least 2 different repeating units selected from the group consisting of (Ill), (VI), (VIII) and (IX); wherein c, e, g, and h are independently an integer selected from 0 to 20, with the proviso that at least two of c, e, g and h are different to 0; and z is an integer selected from 5-35; and wherein the sequential order of the respective repeating units of R₅ and the repeating units denoted with the square brackets with the n integer, may be block or randomly present.
Clause 9. The anionic polymer according to any of clauses 1-8, wherein the anionic polymer is of formula (Ib), wherein Z is selected from the group consisting of —CO—(CH₂)q-S—S—(CH₂)r-NH—(R₅)z-, —CO—(CH₂)p-NH—(R₅)z-, and —(R₅)z-; R₅ is a random or block co-polymer comprising at least 2 different repeating units selected from the group consisting of (Ill), (VI), (VIII) and (IX); and wherein c, e, g, and h are independently an integer selected from 0 to 20, with the proviso that at least two of c, e, g and h are different to 0; and z is an integer selected from 5-35; and m is an integer selected from 5 to 250; and n is an integer selected from 3 to 200; with the proviso that the ratio between m:n ranges from 1:8 to 30:1;
and wherein the sequential order of the respective repeating units of R₅ and the repeating units denoted with the square brackets with the n integer, may be block or randomly present.
Clause 10. The anionic polymer according to any of clauses 1-9, wherein the anionic polymer is of formula (Ib), wherein Z is (R₅)z, according to formula (Ib2):

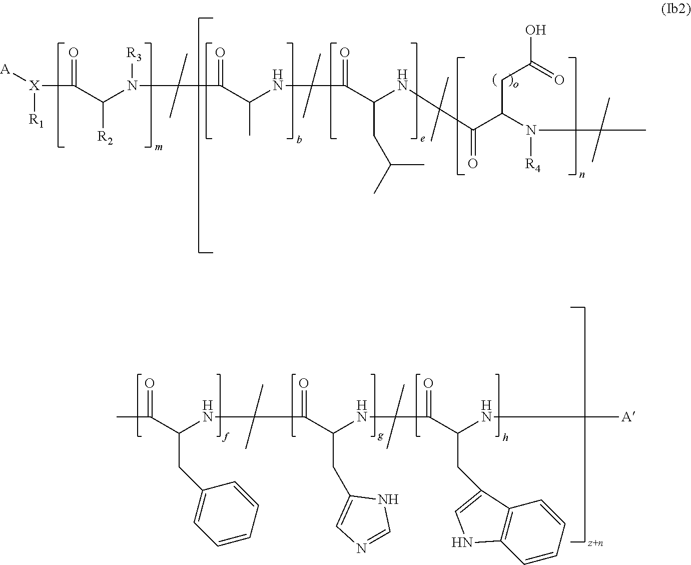

wherein b, e, f, g, and h are each independently an integer selected from 0 to 20; with the proviso that at least two of b, e, f, g, and h is different to 0;
z is an integer selected from 5 to 100;
m is an integer selected from 5 to 250;
n is an integer selected from 3 to 200;
with the proviso that the ratio between m:n ranges from 1:8 to 30:1;
wherein “/” between the repeating units defined by square brackets with the b, e, n, f, g and h integers indicates that the sequential order of the monomer repeating units defined by square brackets on both sides of the sign is arbitrary;
the “/” between the repeating unit defined by square brackets with the m integer and the repeating units defined by the square brackets with the z+n integer, indicates that the sequential order of the monomer repeating units on both sides of the sign is arbitrary.
Clause 11. The anionic polymer according to any of clauses 1-3, is of formula (Ia); R₂ is selected from H, and a radical selected from the group consisting of (X) and (XII); and R₃ is —CH₃.
Clause 12. The anionic polymer according to clause 7, wherein Y is —CO—(CH₂)p-CO—; R₂ is selected from H the group consisting of (X) and (XII); R₃ and R₄ are H; m is an integer selected from 20 to 160; n is an integer selected from 4-100; with the proviso that the ratio between m:n ranges from 1:5 to 10:1.

1. An anionic polymer of formula (Ia) or (Ib), a pharmaceutically acceptable salt thereof, or any stereoisomer or mixtures of stereoisomers, either of the compound of formula (Ia) or (Ib), or of any of its pharmaceutically acceptable salts, comprising homo-polypeptides or random or block or graft co-polypeptides. 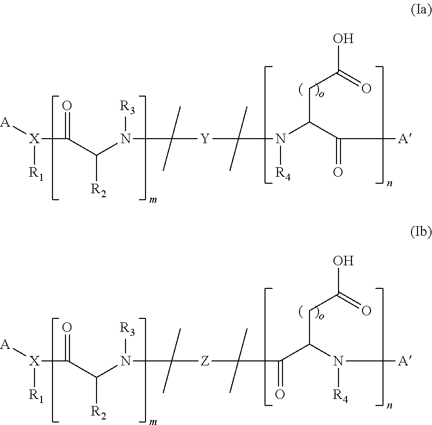
wherein Y is selected from the group consisting of —CO—(CH₂)p-CO— and —CO—(CH₂)q-S—S—(CH₂)r-CO—;
Z is selected from the group consisting of a single bond, —CO—(CH₂)q-S—S—(CH₂)r-NH—(R₅)z-, —CO—(CH₂)p-NH—(R₅)z-, —CO—(CH₂)p-CO—NH—(CH₂)q-S—S—(CH₂)r-NH—(R₅)z- and —(R₅)z-;
o is an integer selected from 1 to 2;
wherein p, q and r are each independently an integer selected from 1 to 6;
R₅ is a random or block co-polymer comprising at least two different repeating units selected from the group consisting of (II), (III), (IV), (V), (VI), (VII), (VIII) and (IX): 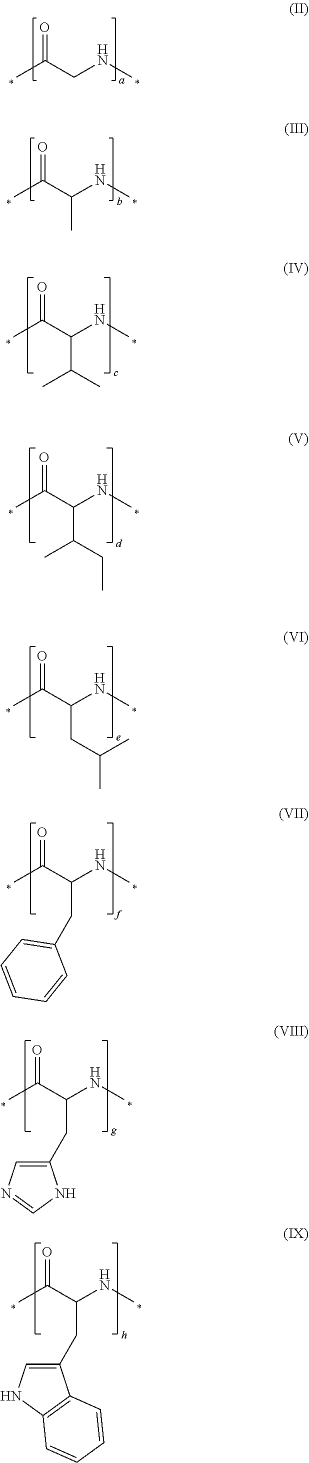
a, b, c, d, e, f, g and h are each independently an integer selected from 0 to 20; with the proviso that at least one of a, b, c, d, e, f, g and h is different to 0;
z is an integer selected from 5 to 100;
X is selected from N, S, and O;
R₁ is selected from the group consisting of H and (C₁-C₆)alkyl, with the proviso that R₁ is absent when X is O;
R₃ and R₄ are each independently selected from the group consisting of H and —CH₃;
m is an integer selected from 5 to 250;
n is an integer selected from 3 to 200;
with the proviso that the ratio between m:n ranges from 1:8 to 30:1;
R₂ is selected from H, and a radical selected from the group consisting of (X), (XI), (XII), and 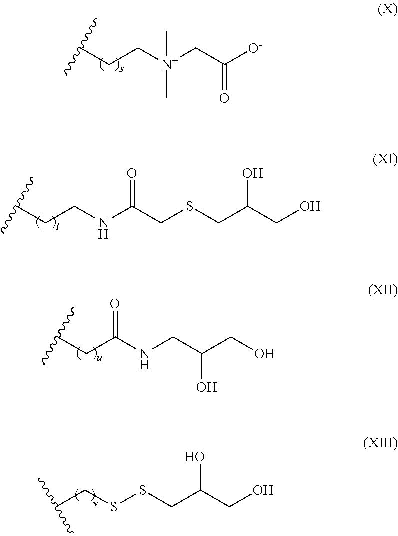
wherein s, t, u, and v are each independently an integer selected from 1 to 4;
wherein “/” indicates that the sequential order of the monomer repeating units defined by square brackets on both sides of the sign is arbitrary, thus though the repeating units defined by square brackets in the formulas Ia and Ib are shown in a particular order for convenience of description, the repeating units may be present in any order and the repeating units may be block or randomly present;
wherein in formula Ib, the sequential order of the respective repeating units of formula (II), (III), (IV), (V), (VI), (VII), (VIII) and (IX) of R₅ and the repeating units denoted with the square brackets with the n integer, may be block or randomly present;
A and A′ are each independently selected from H, OH, straight chain or branched —(C₁-C₆)alkyl, straight chain or branched —CO(C₁-C₆)alkyl, —(C₅-C₁₀)aryl, —(C₅-C₁₀)heteroaryl, —(C₆-C₁₀)aralkyl, —(C₁-C₆)alkyl-O—(C₅-C₁₀)aryl, —(C₅-C₁₀)heterocycloalkyl, —(C₁-C₁₀)alkoxy, —(C₆-C₁₀)aryloxy, —(C₆-C₁₀)aralkoxy, —(C₅-C₁₀)heteroaralkoxy, —(C₁-C₁₀)alkyl-O—(C₆-C₁₀)aryloxy, an amine protecting group, a natural or unnatural alpha amino acid, a carboxyl protecting group, a labelling or imaging agent, and a cell-targeting agent;
being each of A and A′ independently optionally substituted by one or more groups selected from the group consisting of —OH, halogen, —CF₃, —NH₂, —NH—(C₁-C₄)alkyl, —NR₆R₇, —NH—CO—(C₁-C₆)alkyl, —(C₁-C₆)alkyl, —NO₂, —N₃, —CO—(C₁-C₆)alkyl, —CO—O—(C₁-C₆)alkyl, —SO₃H, —SO₂NH₂, —SO₂—N((C₁-C₆)alkyl)₂, —COOH, CONH₂, and —CON((C₁-C₆)alkyl)₂; and
R₆ and R₇ are independently selected from the group consisting of H, —(C1-C₄)alkyl and an amine protecting group.

2. The anionic polymer according to claim 1, wherein m is an integer selected from 20 to 160, and n is an integer selected from 4 to 100; with the proviso that the ratio between m:n ranges from 1:5 to 10:1.

3. The anionic polymer according to claim 1, which is of formula (Ia) or (Ib), R₃ is —CH₃; and R₂ is H.

4. The anionic polymer according to claim 1, which is of formula (Ib), wherein Z is a single bond; R₂ is selected from H, and a radical selected from the group consisting of (X), (XI), (XII), and (XIII); and R₃ is —CH₃.

5. The anionic polymer according to claim 1, wherein the anionic polymer is of formula Ib; Z is selected from the group consisting of —CO—(CH₂)q-S—S—(CH₂)r-NH—(R₅)z-, —CO—(CH₂)p-NH—(R₅)z-, —CO—(CH₂)p-CO—NH—(CH₂)q-S—S—(CH₂)r-NH—(R₅)z- and —(R₅)z-; and R₅ is a random or block co-polymer comprising at least 2 repeating units selected from the group consisting of (III), (VI), (VIII), and (IX); and wherein c, e, g, and h are independently an integer selected from 1 to 20; and z is an integer selected from 5 to 35.

6. The anionic polymer according to claim 1, wherein the anionic polymer is of formula (Ia); R₂ is selected from Hand a radical selected from the group consisting of (X) and (XII); and R₃ is —CH₃.

7. The anionic polymer according to claim 1, wherein the anionic polymer is of formula (Ia): wherein Y is —CO—(CH₂)p-CO—; R₂ is selected from H and a radical selected from the group consisting of (X) and (XII); R₃ and R₄ are H; m is an integer selected from 20 to 160; n is an integer selected from 4 to 100; with the proviso that the ratio between m:n ranges from 1:5 to 10:1.

8. A polymer complex comprising
a) a cationic polymer which covalently or electrostatically bonds with at least one or more active agents selected from the group consisting of pharmaceutically active agents, veterinary active agents, cosmetically active agents, diagnostically active agents, nucleic acids, peptides, antibodies, aptamers, proteins, and mixtures thereof, wherein the cationic polymer and the at least one active agent are forming a positively charged nanoparticle; and
b) at least one anionic polymer of formula (Ia) or (Ib) according to claim 1 which interacts electrostatically with the nanoparticle.

9. The polymer complex according to claim 8, wherein the at least one active agent is selected from the group consisting of low molecular weight drugs, peptides, proteins, antibodies, nucleic acids, aptamers, and combinations thereof.

10. The polymer complex according to claim 9, wherein the nucleic acid is selected from the group consisting of a DNA/RNA hybrid, a short interfering RNA (siRNA), a microRNA (miRNA), sgRNA, a donor DNA, a self-amplifying/replicating RNA, circular RNA (oRNA), a plasmid DNA (pDNA), a closed-linear DNA (cIDNA), a short hairpin RNA (shRNA), a messenger RNA (mRNA), an antisense RNA (aRNA), a messenger RNA (mRNA), a CRISPR guide RNA, an antisense nucleic acid, a decoy nucleic acid, an aptamer, and a ribozyme.

11. The polymer complex according to claim 10, wherein the nucleic acid is a cIDNA.

12. A protein-based complex comprising
a) a cationically charged protein; and
b) at least one anionic polymer of formula (Ia) or (Ib) according to claim 1 which interacts electrostatically with the protein;
or alternatively
a′) an anionically charged protein covalently or electrostatically bound to a cationic polymer forming a positively charged nanoparticle; and
b′) at least one anionic polymer of formula (Ia) or (Ib) according to claim 1 which interacts electrostatically with the positively charged nanoparticle of a′).

13. A composition comprising at least one polymer complex as defined in claim 1, together with one or more pharmaceutically, diagnostically, veterinary, or cosmetically acceptable excipients or carriers.

14. A method of using a therapeutic product for medicine, wherein the therapeutic product is a polymer complex according to claim 8.

15. A method of delivering a nucleic acid or a protein into a cell, tissue, or extracellular space, wherein the method comprises using a device comprising the polymer complex according to claim 1.

16. A method of delivering a nucleic acid or protein into a target cell, tissue or extracellular space, which comprises contacting a solution that contains the polymer complex according to claim 8, with the target cell, tissue or extracellular space, so that the complex can be introduced into the target cell, tissue or extracellular space; transferring the complex from an endosome of the target cell to cytoplasm of the target cell; dissociating the complex in the target cell; and releasing the nucleic acid or protein into the cytoplasm.

17. A method of transfecting a cell comprising contacting the cell with the polymer complex according to claim 8.


SEQUENCE LISTING (1 sequence)

SEQ ID NO: 1 - DNA, 5580 bp, synthetic construct
  misc_feature 1..5580: note = clDNA
  source 1..5580: mol_type = other DNA
gcgtataatggactattgtgtgctgataggtctcgccctgatttaaatcgtcgaccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggcccgggatccaccggtcgccaccatggaagacgccaaaaacataaagaaaggcccggcgccattctatcctctagaggatggaaccgctggagagcaactgcataaggctatgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaattcctctggatctactgggttacctaagggtgtggcccttccgcatagaactgcctgcgtcagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaagtgcgttgctagtaccaaccctattttcattcttcgccaaaagcactctgattgacaaatacgatttatctaatttacacgaaattgcttctgggggcgcacctctttcgaaagaagtcggggaagcggttgcaaaacgcttccatcttccagggatacgacaaggatatgggctcactgagactacatcagctattctgattacacccgagggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatggctacattctggagacatagcttactgggacgaagacgaacacttcttcatagttgaccgcttgaagtctttaattaaatacaaaggatatcaggtggcccccgctgaattggaatcgatattgttacaacaccccaacatcttcgacgcgggcgtggcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagtccaaattgtaaagcggccgcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttagtcgaccatttaaatcggtctcgagggtatcagcacacaattgcccattatacgcgcgtataatgggcaattgtgtgctgataccctcgagaccgatttaaatggtcgactaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatccccgcggccgctttacaatttggactttccgcccttcttggcctttatgaggatctctctgatttttcttgcgtcgagttttccggtaagacctttcggtacttcgtccacaaacacaactcctccgcgcaactttttcgcggttgttacttgactggcgacgtaatccacgatctctttttccgtcatcgtctttccgtgctccaaaacaacaacggcggcgggaagttcaccggcgtcatcgtcgggaagacctgccacgcccgcgtcgaagatgttggggtgttgtaacaatatcgattccaattcagcgggggccacctgatatcctttgtatttaattaaagacttcaagcggtcaactatgaagaagtgttcgtcttcgtcccagtaagctatgtctccagaatgtagccatccatccttgtcaatcaaggcgttggtcgcttccggattgtttacataaccggacataatcataggtcctctgacacataattcgcctctctgattaacgcccagcgttttcccggtatccagatccacaaccttcgcttcaaaaaatggaacaactttaccgaccgcgcccggtttatcatccccctcgggtgtaatcagaatagctgatgtagtctcagtgagcccatatccttgtcgtatccctggaagatggaagcgttttgcaaccgcttccccgacttctttcgaaagaggtgcgcccccagaagcaatttcgtgtaaattagataaatcgtatttgtcaatcagagtgcttttggcgaagaatgaaaatagggttggtactagcaacgcactttgaattttgtaatcctgaagggatcgtaaaaacagctcttcttcaaatctatacattaagacgactcgaaatccacatatcaaatatccgagtgtagtaaacattccaaaaccgtgatggaatggaacaacacttaaaatcgcagtatccggaatgatttgattgccaaaaataggatctctggcatgcgagaatctgacgcaggcagttctatgcggaagggccacacccttaggtaacccagtagatccagaggaattcattatcagtgcaattgttttgtcacgatcaaaggactctggtacaaaatcgtattcattaaaaccgggaggtagatgagatgtgacgaacgtgtacatcgactgaaatccctggtaatccgttttagaatccatgataataattttctggattattggtaattttttttgcacgttcaaaattttttgcaacccctttttggaaacaaacactacggtaggctgcgaaatgttcatactgttgagcaattcacgttcattataaatgtcgttcgcgggcgcaactgcaactccgataaataacgcgcccaacaccggcataaagaattgaagagagttttcactgcatacgacgattctgtgatttgtattcagcccatatcgtttcatagcttctgccaaccgaacggacatttcgaagtattccgcgtacgtgatgttcacctcgatatgtgcatctgtaaaagcaattgttccaggaaccagggcgtatctcttcatagccttatgcagttgctctccagcggttccatcctctagaggatagaatggcgccgggcctttctttatgtttttggcgtcttccatggtggcgaccggtggatcccgggccgcgggtacaattccgcagcttttagagcagaagtaacacttccgtacaggcctagaagtaaaggcaacatccactgaggagcagttctttgatttgcaccaccaccggatccgggacctgaaataaaagacaaaaagactaaacttaccagttaactttctggtttttcagttcctcgagtaccggatcctctagagtccggaggctggatcggtcccggtgtcttctatggaggtcaaaacagcgtggatggcgtctccaggcgatctgacggttcactaaacgagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacggtcgacgatttaaatcagggcgagacctatcagcacacaatagtccattatacgc